(19) United States
(12) Patent Application Publication
(10) Pub. No.: US 2016/0166637 A1
(43) Pub. Date: Jun. 16, 2016

(54) METHODS OF TREATING A CANCER THROUGH TARGETED DISRUPTION OF ALPHA CONNEXIN 43-ZONULA OCCLUDENS-1 (ZO-1) INTERACTION

(71) Applicants: Robert G. GOURDIE, Roanoke, VA (US); Zhi SHENG, Roanoke, VA (US)

(72) Inventors: Robert G. Gourdie, Roanoke, VA (US); Zhi Sheng, Roanoke, VA (US)

(73) Assignee: Virginia Tech Intellectual Properties, Inc., Blacksburg, VA (US)

(21) Appl. No.: 14/909,674

(22) PCT Filed: Jun. 16, 2014
(86) PCT No.: PCT/US14/42528

§ 371 Date: Feb. 2, 2016

Related U.S. Application Data
(60) Provisional application No. 61/861,686, filed on Aug. 2, 2013.

(51) Int. Cl. A61K 38/17; A61K 31/495; A61K 45/06

(52) U.S. Cl. CPC A61K 38/177; A61K 45/06; A61K 31/495

(57) ABSTRACT

The present disclosure describes methods of treating a cancer based on the targeted disruption of alpha connexin 43-zonula occludens-1 (ZO-1) interaction. The methods may include administering to a subject an effective amount of a composition comprising a peptide with a contiguous sequence of amino acids representing a portion of the carboxy terminus of an alpha connexin protein or conservative variant thereof, wherein said carboxy terminus includes the sequence up to the transmembrane domain, optionally in combination with administration of a chemotherapeutic agent. In embodiments, the cancer is glioma and the chemotherapeutic agent is temozolomide. The methods may also include administration of vectors encoding the peptide or host cells comprising the vectors. Also described are compositions for treating a cancer comprising one or more peptides, nucleic acids, vectors, and/or host cells, optionally in combination with a chemotherapeutic agent such as temozolomide.

### CROSS-REFERENCE TO RELATED APPLICATIONS

This application relies on the disclosure of and claims priority to and the benefit of the filing date of U.S. Provisional Application No. 61/861,686, filed Aug. 2, 2013, the disclosure of which is hereby incorporated by reference herein in its entirety.

### REFERENCE TO SEQUENCE LISTING SUBMITTED IN COMPUTER READABLE FORM

The present application contains a Sequence Listing which has been submitted in ASCII format by way of EFS-Web and is hereby incorporated by reference herein in its entirety. The ASCII file was created May 28, 2014 and named VTIP86PCTsequence, which is 32.2 kilobytes in size and which is identical to the paper copy filed with this application.

### BACKGROUND OF THE INVENTION

1. Field of the Invention

The present disclosure relates to the field of cancer treatment. More particularly, the present invention relates to methods of treating a cancer through targeted disruption of alpha connexin 43-zonula occludens-1 (ZO-1) interaction. In embodiments, the methods relate to treatment of glioma through administration of a therapy that disrupts alpha connexin 43-zonula occludens-1 (ZO-1) interaction in combination with a cancer therapeutic agent such as temozolomide.

2. Description of Related Art

Glioblastoma multiforme (GBM) is the most common and most malignant of the glial tumors. In North America, the incidence is approximately 2-3 new cases per 100,000 people per year, and is slightly more common in men than women. Common presenting symptoms of GBM include slow progressive neurological deficit and symptoms, increased intracranial pressure including headaches, nausea and vomiting, and cognitive impairment, which symptoms are usually present for three months or less. The tumor is typically diagnosed through imaging studies of the brain, which may include computed tomography, magnetic resonance imaging, positron emission tomography, and magnetic resonance spectroscopy.

If patients are not treated, they typically will die within 3 months, and patients on optimal therapy have a median survival of approximately 12 months. Less than 25% survive up to 2 years and less than 10% survive up to 5 years. Optimal therapy for GBM consists of maximal surgical resection, radiotherapy, and concomitant and adjuvant chemotherapy, which can boost survival in more than 25% of patients. Temozolomide has become the standard adjuvant chemotherapy, although other chemotherapeutic agents have been attempted including nitrosoureas, cisplatin, bevacizumab, and tyrosine kinase inhibitors.

The infiltrative nature of glioma cells in the brain makes this an extremely difficult disease to treat, as can be seen in the MRI of the brain of a glioma patient shown in FIG. 1. Three months after the tumor was surgically removed, metastatic lesions occurred, and one month after that, the new tumor mass progressed significantly, and there was also some local recurrence. Thus, there is a need in the art for improved glioma and other cancer treatments.

### SUMMARY OF THE INVENTION

Embodiments of this disclosure provide methods of treating a cancer comprising administering to the subject one or more compositions (e.g., polypeptides, nucleic acids, vectors, and/or host cells) in a pharmaceutically acceptable carrier. In embodiments, the compositions may comprise a peptide comprising a carboxy-terminal amino acid sequence of an alpha Connexin, a nucleic acid encoding such peptide, a vector containing such nucleic acid, or a host cell containing such vector. Such compounds and compositions can include for example those disclosed in International Patent Application Publication No. WO2006/069181. Further, embodiments of the methods may include administering one or more chemotherapeutic agents in combination with the compositions, or as part of the compositions.

In one embodiment, the present disclosure provides a method of treating or preventing a cancer in a subject, comprising administering to the subject an effective amount of a composition comprising a peptide consisting of a contiguous sequence of amino acids representing a portion of the carboxy terminus of an alpha connexin protein or conservative variant thereof, wherein said carboxy terminus includes the sequence up to the transmembrane domain. As used in the context of this disclosure, a “portion” can refer to the entire full length protein or only a portion thereof. Preferably, when a polypeptide or peptide of the invention comprises a portion of the carboxy terminus of an alpha connexin protein or conservative variant thereof, the polypeptide or peptide does not comprise the full length alpha connexin protein and only a portion thereof.

In another embodiment, the present disclosure provides a method of treating or preventing a cancer in a subject, comprising administering to the subject an effective amount of a composition comprising a vector comprising a nucleic acid sequence encoding a peptide consisting of a contiguous sequence of amino acids representing a portion of the carboxy terminus of an alpha connexin protein or conservative variant thereof, wherein said carboxy terminus includes the sequence up to the transmembrane domain.

In another embodiment, the present disclosure provides a method of treating or preventing a cancer in a subject, comprising administering to the subject an effective amount of a composition comprising a host cell comprising a vector comprising a nucleic acid sequence encoding a peptide consisting of a contiguous sequence of amino acids representing a portion of the carboxy terminus of an alpha connexin protein or conservative variant thereof, wherein said carboxy terminus includes the sequence up to the transmembrane domain.

In embodiments, a cancer therapeutic agent may be administered with any composition described here, either separately or as a part of the composition. The cancer therapeutic agent may be any form of radiation used to treat cancer or may be any chemotherapeutic agent described herein or those not described or that become known after filing of this disclosure. In one particular embodiment, the cancer therapeutic agent is temozolomide. However, other cancer therapeutic agents, including those described in this disclosure, may also be administered

In embodiments, any cancer may be treated. In one particular embodiment, a high grade astrocytoma such as glioblastoma or glioma may be treated. However, other cancers, including those described in this disclosure, may be treated.

In embodiments, methods of this disclosure may treat a cancer with a vector comprising a nucleic acid sequence encoding a chimeric polypeptide comprising the following components (optionally listed in the order of the amino terminus to the carboxy terminus of the chimeric polypeptide):

a secretory signal peptide;

an Fc fragment;

IL13 peptide;

an MMP cleavage domain;

a cellular internalization peptide; and

a fragment of an alpha connexin protein;

wherein the fragment of an alpha connexin protein represents a contiguous sequence of amino acids representing a portion of the carboxy terminus of an alpha connexin protein or conservative variant thereof wherein said carboxy terminus includes the sequence up to the transmembrane domain.

In embodiments, the present disclosure provides a method of treating or preventing cancer in a subject, comprising administration to the subject a composition comprising a compound which disrupts alpha connexin 43 interaction with ZO-1.

In embodiments, compounds of this disclosure which disrupt alpha connexin 43 interaction with ZO-1 are therapeutic nucleotides designed to inhibit expression of alpha-connexin 43. In particular embodiments, the therapeutic nucleotide may be an antisense oligonucleotide, an siRNA, a ribozyme, an RNA external guide sequence, a shRNA, an aptamer, or a miRNA.

In embodiments, the compound of this disclosure which disrupts alpha connexin 43 interaction with ZO-1 has binding activity for the carboxy terminus amino acid sequence of alpha connexin 43 or a conservative variant thereof. In particular embodiments, the compound that has binding activity for the carboxy terminus amino acid sequence of alpha connexin 43 or a conservative variant thereof may be ACT-1 peptide, H2 peptide, AAP10, GAP19, GAP134, ZP123, danepeptide, rotigaptide, RXP-E, AAP10, or an antibody with binding activity specific to a carboxy terminal amino acid sequence of alpha connexin 43.

In embodiments, the compound of this disclosure which disrupts alpha connexin 43 interaction with ZO-1 is an alpha connexin 43 function inhibitor. In particular embodiments, the compound that is an alpha connexin 43 function inhibitor may be JM peptide, GAP26, GAP27, heptanol, octanol anesthetics; halothane, propofol, ethflurane, flufenamic acid, 18-beta-glycyrrhetinic acid, and derivatives thereof; lysophosphatidic acid; lindane; mefloquine; okadaic acid; oleamide; quinidine; quinine; all trans-retinoic acid; vitamin A and retinoic acid derivatives or tamoxifen.

In embodiments, provided are compositions comprising a peptide consisting of a contiguous sequence of amino acids representing a portion of the carboxy terminus of an alpha connexin protein or conservative variant thereof and temozolomide, wherein said carboxy terminus includes the sequence up to the transmembrane domain. In embodiments, the polypeptide is an isolated polypeptide.

In another embodiment, the present disclosure provides a chimeric polypeptide comprising the following components (optionally listed in order from the amino terminus to the carboxy terminus):

a secretory signal peptide;

an Fc fragment;

IL13 peptide;

an MMP cleavage domain;

a cellular internalization peptide; and

a fragment of an alpha connexin protein;

wherein the fragment of an alpha connexin protein represents a contiguous sequence of amino acids representing a portion of the carboxy terminus of an alpha connexin protein or conservative variant thereof wherein said carboxy terminus includes the sequence up to the transmembrane domain.

In another embodiment, the present disclosure provides a nucleic acid encoding the chimeric polypeptide, which can be an isolated nucleic acid.

In another embodiment, the present disclosure provides a vector comprising the nucleic acid encoding the chimeric polypeptide.

In embodiments, the vector further comprises one or more miR1 and miR-122 binding sites.

In embodiments, the vector comprises a promoter located 5′ from the nucleic acid sequence, and expression of the peptide is regulated by the promoter. The promoter may be a constitutive promoter or an inducible promoter. The inducible promoter may be activated by doxycycline.

In another embodiment, the present disclosure provides a host cell comprising the vector comprising the nucleic acid encoding the chimeric polypeptide, such as a host cell that is a mesenchymal stem cell.

In another embodiment, the present disclosure provides a composition comprising the chimeric polypeptide, vector, or host cell. In embodiments, the composition may comprise a liposome formulation of the peptide or vector. In embodiments, the composition may comprise a ligand for the IL-13 α2 receptor such as is IL-13, PEP-1, or Chitinase 3-like-1.

In another embodiment, the present disclosure provides a method of treating or preventing a glioma in a subject, comprising administering to the subject an effective amount of a composition comprising a peptide consisting of a contiguous sequence of amino acids representing a portion of the carboxy terminus of an alpha connexin protein or conservative variant thereof and temozolomide, wherein said carboxy terminus includes the sequence up to the transmembrane domain.

In embodiments, a peptide of this disclosure consists of the carboxy terminal most 3 to 120 contiguous amino acids from the alpha connexin protein. In other embodiments, a peptide of this disclosure consists of the carboxy terminal most 4 to 30 contiguous amino acids from the alpha connexin protein. In other embodiments, a peptide of this disclosure consists of the carboxy terminal most 5 to 19 contiguous amino acids from the alpha connexin protein. In other embodiments, a peptide of this disclosure consists of the last 9 contiguous amino acids of the carboxy terminus of the alpha connexin protein.

In embodiments, the alpha connexin is selected from the group consisting of Connexin 30.2, Connexin 31.9, Connexin 33, Connexin 35, Connexin 36, Connexin 37, Connexin 38, Connexin 39, Connexin 39.9, Connexin 40, Connexin 40.1, Connexin 43, Connexin 43.4, Connexin 44, Connexin 44.2, Connexin 44.1, Connexin 45, Connexin 46, Connexin 46.6, Connexin 47, Connexin 49, Connexin 50, Connexin 56, and Connexin 59.

In embodiments, the alpha Connexin is Connexin 37, Connexin 40, Connexin 43, or Connexin 45.

In embodiments, a peptide of this disclosure comprises the amino acid sequence selected from the group consisting of SEQ ID NO: 1, SEQ ID NO: 2, SEQ ID NO: 3, SEQ ID NO: 4, SEQ ID NO: 5, and SEQ ID NO:43.

In embodiments, a peptide of this disclosure comprises the amino sequence of SEQ ID NO: 2.

In embodiments, a peptide of this disclosure has 1 to 5 conservative substitutions as compared to a polypeptide having the sequence of SEQ ID NO:2.

In embodiments, a peptide can have an amino acid sequence with at least 65% sequence identity to the c-terminal most 9 amino acids of SEQ ID NO:1.

In embodiments, a peptide of this disclosure comprises an amino acid sequence with at least 75% sequence identity to the c-terminal most 9 amino acids of SEQ ID NO:1.

In embodiments, a peptide of this disclosure comprises an amino acid sequence with at least 85% sequence identity to the c-terminal most 9 amino acids of SEQ ID NO:1

In embodiments, a peptide of this disclosure further comprises a cellular internalization sequence.

In embodiments, the cellular internalization sequence comprises an amino acid sequence of a protein selected from a group consisting of Antennapedia, TAT, HIV-Tat, Penetratin, Antp-3A (Antp mutant), Buforin II, Transportan, MAP (model amphipathic peptide), K-FGF, Ku70, Prion, pVEC, Pep-1, SynB 1, Pep-7, HN-1, BGSC (Bis-Guanidinium-Spermidine-Cholesterol) and BGTC (Bis-Guanidinium-Tren-Cholesterol).

In embodiments, the cellular internalization sequence is Antennapedia, and wherein the sequence comprises the amino acid sequence of SEQ ID NO:7.

In embodiments, the peptide is linked at its amino terminus to the cellular internalization transporter sequence, and wherein the amino acid sequence of the polypeptide and cellular transporter sequence is selected from the group consisting of SEQ ID NO:8, SEQ ID NO:9, SEQ ID NO:10, SEQ ID NO:11, and SEQ ID NO:12.

In embodiments, the peptide is linked at its amino terminus to the cellular internalization transporter sequence, and wherein the amino acid sequence of the polypeptide and cellular transporter sequence has at least 88% sequence identity to SEQ ID NO:8, SEQ ID NO:9, SEQ ID NO:10, SEQ ID NO:11, or SEQ ID NO:12.

Additional embodiments and aspects of these embodiments and their advantages may be found in the foregoing drawings and detailed description, which are intended to be illustrative of the invention rather than limiting.

### BRIEF DESCRIPTION OF THE DRAWINGS

The accompanying drawings illustrate certain aspects of embodiments of the present invention, and should not be used to limit or define the invention. Together with the written description the drawings explain certain principles of the invention.

FIG. 1 is a set of MRI images of a glioma patient.

FIG. 2 is a schematic diagram showing the generic structure of connexin 43 and ZO-1 and the interaction between connectin-43 and ZO-1.

FIG. 3 is a schematic diagram showing the generic structure of ACT-1.

FIG. 4 is a schematic diagram showing ACT-1 inhibition of Cx43-ZO-1 interaction.

FIG. 5 is an image showing the results of a dot blot assay for testing binding of AAP10 and AAP10-pTyr with Connexin43-carboxy terminus (CT).

FIG. 6 is an image of a polyacrylamide gel showing EDAC cross-linking of ACT-1 and Cx43-CT.

FIGS. 7A-7C are images of an aggregation experiment using U87MG cells showing no treatment (FIG. 7A), 25 μM reverse sequence peptide (control peptide) (FIG. 7B), and 25 μM ACT-1(FIG. 7C).

FIG. 7D is a graph showing aggregation index calculations for the aggregation experiment using U87MG cells showing no treatment, 1 μM ACT-1, 25 μM ACT-1, 1 μM reverse sequence peptide (control peptide), and 25 μM reverse sequence peptide (control peptide).

FIGS. 8A-8C are images of an aggregation experiment using connexin-deficient C6 glioma cells showing no treatment (FIG. 8A), 25 μM reverse sequence peptide (control peptide) (FIG. 8B), and 25 μM ACT-1 (FIG. 8C).

FIG. 8D is a graph showing aggregation index calculations for the aggregation experiment using connexin-deficient C6 glioma cells showing no treatment, 1 μM ACT-1, 25 μM ACT-1, 1 μM reverse sequence peptide (control peptide), and 25 μM reverse sequence peptide (control peptide).

FIGS. 9A-9C are images of a motility experiment using U87MG cells showing no treatment (FIG. 9A), 25 μM reverse sequence peptide (control peptide) (FIG. 9B), and 25 μM ACT-1 (FIG. 9C).

FIG. 9D is a graph showing the results of the motility experiment using U87MG cells showing no treatment, 1 μM ACT-1, 25 μM ACT-1, 1 μM reverse sequence peptide (control peptide), and 25 μM reverse sequence peptide (control peptide).

FIGS. 10A-10C are images of a motility experiment using C6 cells stably expressing Cx43 showing no treatment (FIG. 10A), 25 μM reverse sequence peptide (control peptide) (FIG. 10B) and 25 μM ACT-1 (FIG. 10C).

FIG. 10D is a graph showing the results of the motility experiment using C6 cells stably expressing Cx43 showing no treatment, 1 μM ACT-1, 25 μM ACT-1, 1 μM reverse sequence peptide (control peptide), and 25 μM reverse sequence peptide (control peptide).

FIGS. 11A-11C are images of a motility experiment using wild type C6 cells showing no treatment (FIG. 11A), 25 μM reverse sequence peptide (control peptide) (FIG. 11B) and 25 μM ACT-1 (FIG. 11C).

FIG. 11D is a graph showing the results of the motility experiment using wild type C6 cells showing no treatment, 1 μM ACT-1, 25 μM ACT-1, 1 μM reverse sequence peptide (control peptide), and 25 μM reverse peptide (control peptide).

FIG. 12A is an image showing a glioma cell aggregate formed by hanging drop sedimentation and FIG. 12B is an image showing a glioma spheroid implanted into 3D collagen gel. FIGS. 12C and 12D are images of the implant after 48 hours.

FIG. 13 is a graph showing the effects of ACT-1 treatment at concentrations from 30 to 120 μM on the viability of the glial stem cell (GSC) line GS9-6 with or without temozolomide.

FIG. 14 is a graph showing the effects of 120 μM ACT-1 and 100 μM TMZ individually or in combination on the viability of the human U87MG glioblastoma cell line.

FIG. 15A-15F are images show the following treatments of GS9-6 glioma stem cell cultures: untreated GS9-6 cells (FIG. 15A), GS9-6 cells treated with ACT-1 (FIG. 15B), GS9-6 cells treated with rACT-1 (reverse sequence control) (FIG. 15C), GS9-6 cells treated with TMZ (FIG. 15D), GS9-6 cells treated with TMZ and ACT-1 (FIG. 15E), GS9-6 cells treated with rACT-1 and TMZ (FIG. 15F).

FIG. 16 is a graph showing the effects of 50 μM TMZ, 100 μM ACT-1, and 100 μM reverse sequence control peptide on GS9-6 cells ability for self-renewal.

FIG. 17 is a graph showing the effects of 10 μM ACT-1 and 100 μM TMZ individually or in combination on GS9-6 cells ability for self-renewal.

FIG. 18 is an image of a Western blot showing connexin 43 (GJA1) and beta-actin (ACTB) expression in LN229/GSCs compared to parental LN229 cells.

FIG. 19 is a graph of tumor volume over 6 weeks in a glioma xenograft mouse model for untreated, 100 mg/kg ACT-1 treated, 7.5 mg/kg TMZ treated, and combinatorial 100 mg/kg ACT-1 and 7.5 mg/kg TMZ treated mice.

FIG. 20 is graph of tumor volume at 37 days in a glioma xenograft mouse model for untreated, 100 mg/kg ACT-1 treated, 7.5 mg/kg TMZ treated, and combinatorial 100 mg/kg ACT-1 and 7.5 mg/kg TMZ treated mice.

FIG. 21 is a graph of tumor volume over 53 days in a glioma xenograft mouse model for untreated, 200 μM/tumor ACT-1, 100 mg/kg TMZ treated, and combinatorial 200 μM/tumor ACT-1 and 100 mg/kg TMZ treated mice

FIG. 22 is a graph of tumor volume at 53 days in a glioma xenograft mouse model for untreated, 200 μM/tumor ACT-1, 100 mg/kg TMZ treated, and combinatorial 200 μM/tumor ACT-1 and 100 mg/kg TMZ treated mice.

FIG. 23 is an image showing excised tumors (left and right tumor) from a glioma xenograft mouse model for untreated, 200 μM/tumor ACT-1, 100 mg/kg TMZ treated, and combinatorial 200 μM/tumor ACT-1 and 100 mg/kg TMZ treated mice.

FIG. 24A is a bright field image of U251 xenograft and FIG. 24B is a bright field image of contralateral brain, while FIG. 24C is a TAMRA image of U251 xenograph and FIG. 24D is a TAMRA image of contralateral brain. The TAMRA and interleukin 13 (IL13) peptide-conjugated nanoparticles were intravenously injected into mice harboring U251 glioblastoma.

FIG. 25 is a schematic diagram showing the generic structure of ACT-1 chimeric polypeptide.

FIG. 26 is a schematic diagram showing the generic structure of an rAAV vector engineered to express a chimeric ACT-1 protein.

FIG. 27 is a pLVX-TRE3G-IRES plasmid map with the chimeric ACT-1 gene inserted into MCS-1 site and EGFP gene inserted into the MCS-2 site.

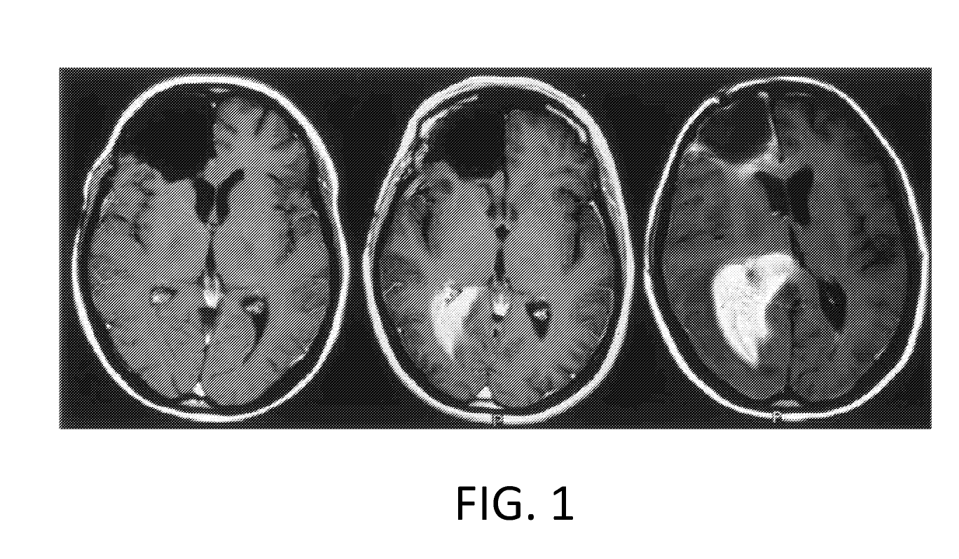

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

Abbreviations used in this specification include the following: TMZ=temozolomide; ACT=alpha Connexin carboxy-terminal; ZO-1=zonula occludens-1; MSC=mesenchymal stem cells; GBM=glioblastoma multiforme; Cx=Connexin; IL-13=Interleukin 13; AAP10=antiarrhythmic peptide 10; GSC=glioma stem cells; EGFP=enhanced green fluorescent protein; CT=carboxy terminus; Tet=tetracycline; Dox=doxycycline; MCS=multiple cloning site. Abbreviations not listed here will have their art-recognized meaning.

The disclosed method and compositions may be understood more readily by reference to the following detailed description of particular embodiments and the Examples, and to the Figures and their previous and following description.

In one embodiment, the present disclosure provides a polypeptide comprising a carboxy-terminal amino acid sequence of an alpha Connexin (also referred to herein as an alpha Connexin carboxy-Terminal (ACT) polypeptide), or a conservative variant thereof. In another embodiment, the present disclosure provides a nucleic acid encoding a carboxy-terminal amino acid sequence of an alpha Connexin or a vector containing such nucleic acid. In another embodiment, the present disclosure provides a nucleic acid encoding a therapeutic DNA or RNA molecule designed to inhibit the expression of an alpha Connexin. In another embodiment, the present disclosure provides a monoclonal or polyclonal antibody or antibody fragment for binding to a carboxy-terminal amino acid sequence of an alpha Connexin. Embodiments also provide compositions encoding the peptide, nucleic acid, antibody, or vector.

In one aspect, the provided ACT polypeptide alters glioma cell invasiveness through increased adhesion. In another aspect, the ACT polypeptide alters glioma cell invasiveness through decreased motility. In another aspect, the ACT polypeptide alters glioma cell invasiveness through decreased invasion. In another aspect, the ACT polypeptide alters glioma cell invasiveness through the modification of any combination of adhesion, motility, or invasion. In aspects, the ACT polypeptide sensitizes tumor cells to a chemotherapeutic agent through the bystander effect.

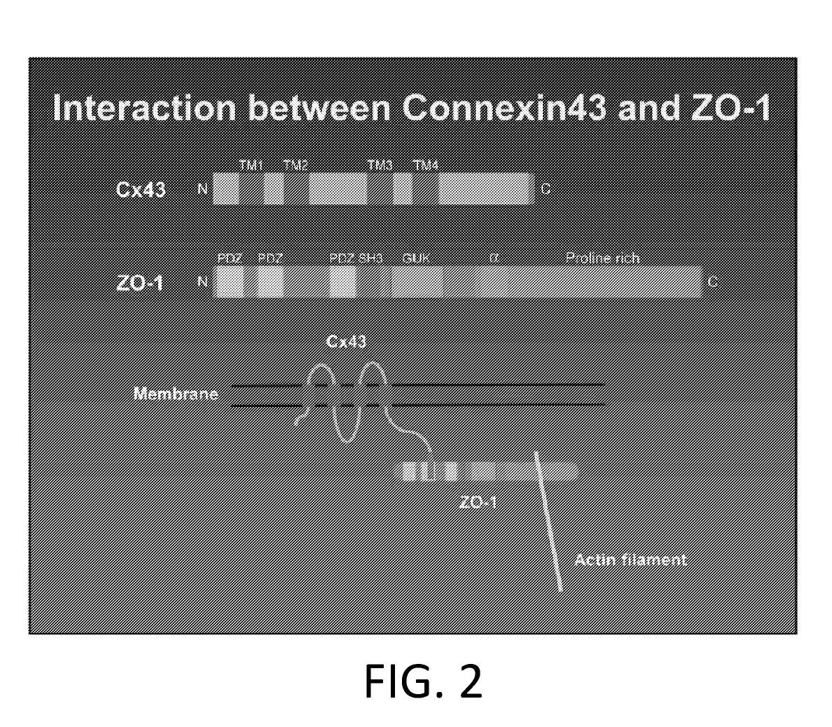

In one embodiment, the present disclosure provides a polypeptide comprising a carboxy-terminal amino acid sequence of alpha Connexin 43. FIG. 2 shows the interaction between Connexin 43 and ZO-1, an important scaffolding protein. FIG. 2 shows the cell membrane, traversed four times by Cx43, the C-terminus of which binds to ZO-1 in the cytoplasm. The proline-rich tail of ZO-1 in turn binds to the actin cytoskeleton. This Cx43-ZO-1 interaction has been shown to be of great importance to GJ organization.

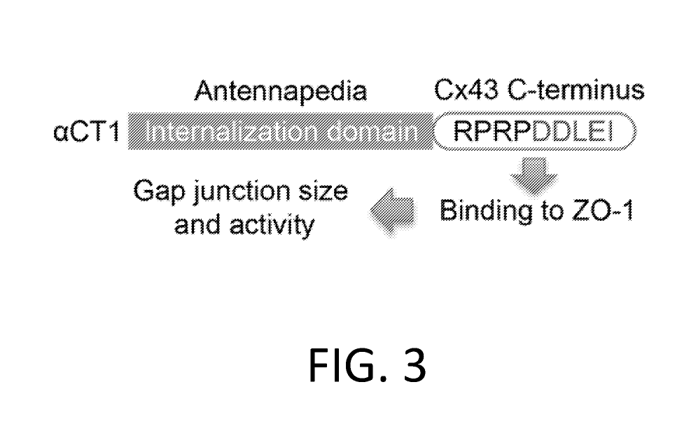

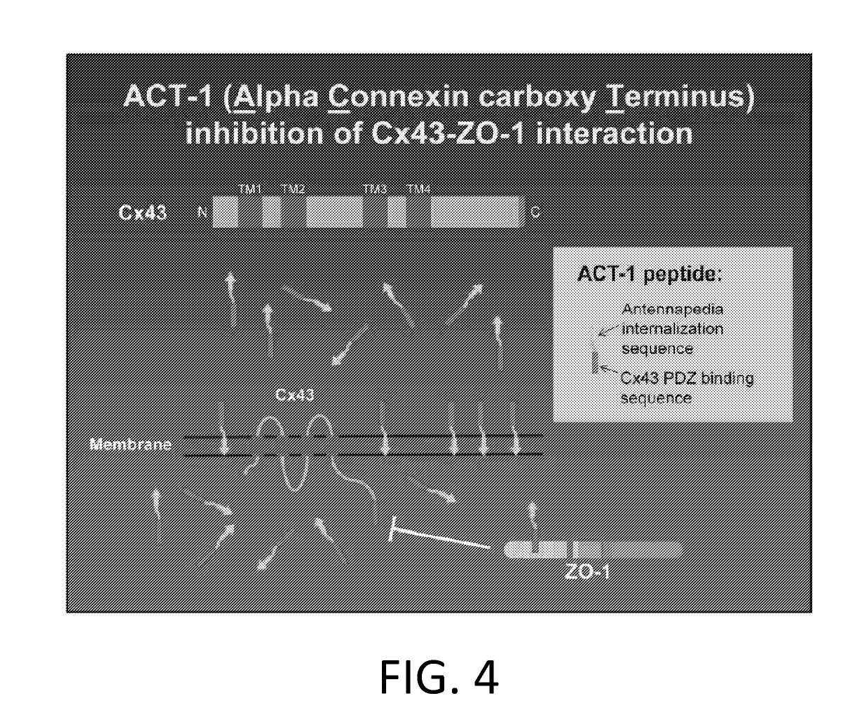

FIG. 3 shows a generic structure an embodiment of a polypeptide of this disclosure (ACT-1). The ACT-1 polypeptide comprises an Antennapedia internalization domain so that is can enter cells and competitively inhibit Cx43-ZO-1 interaction by binding to the second PDZ domain of ZO-1 through the c terminal 9 amino acids of Connexin-43 (SEQ ID NO:2). FIG. 3 shows that the Cx43 c-terminus binds to ZO-1, which ultimately affects gap junction size and activity, and FIG. 4 shows that ACT-1 inhibits interaction of Cx43 to ZO-1.

It is to be understood that the disclosed compositions and methods are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a vector is disclosed and discussed and a number of vector components including the promoters are discussed, each and every combination and permutation of promoters and other vector components and the modifications that are possible are specifically contemplated unless specifically indicated to the contrary.

For example, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of steps that can be performed it is understood that each of these steps can be performed with any specific embodiment or combination of embodiments of the disclosed methods, and that each such combination is specifically contemplated and should be considered disclosed.

A variety of sequences are provided herein and these and others can be found in Genbank at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

The polypeptide of this disclosure can be any polypeptide comprising the carboxy-terminal most amino acids of an alpha Connexin, wherein the polypeptide does not comprise the full-length alpha Connexin protein. Thus, in one aspect, the provided polypeptide does not comprise the cytoplasmic N-terminal domain of the alpha Connexin. In another aspect, the provided polypeptide does not comprise the two extracellular domains of the alpha Connexin. In another aspect, the provided polypeptide does not comprise the four transmembrane domains of the alpha Connexin. In another aspect, the provided polypeptide does not comprise the cytoplasmic loop domain of the alpha Connexin. In another aspect, the provided polypeptide does not comprise that part of the sequence of the cytoplasmic carboxyl terminal domain of the alpha Connexin proximal to the fourth transmembrane domain. There is a conserved proline or glycine residue in alpha Connexins consistently positioned some 17 to 30 amino acids from the carboxyl terminal-most amino acid (Table 2). For example, for human Cx43 a proline residue at amino acid 363 is positioned 19 amino acids back from the carboxyl terminal most isoleucine. In another example, for chick Cx43 a proline residue at amino acid 362 is positioned 18 amino acids back from the carboxyl terminal-most isoleucine. In another example, for human Cx45 a glycine residue at amino acid 377 is positioned 19 amino acids back from the carboxyl terminal most isoleucine. In another example for rat Cx33, a proline residue at amino acid 258 is positioned 28 amino acids back from the carboxyl terminal most methionine. Thus, in another aspect, the provided polypeptide does not comprise amino acids proximal to said conserved proline or glycine residue of the alpha Connexin. Thus, the provided polypeptide can comprise the c-terminal-most 4 to 30 amino acids of the alpha Connexin, including the c-terminal most 4, 5, 6, 7, 8, 9, 10, 11, 12, 13, 14, 15, 16, 17, 18, 19, 20, 21, 22, 23, 24, 25, 26, 27, 28, 29, and/or 30 amino acids of the alpha Connexin.

The carboxy-terminal most amino acids of an alpha Connexin in the provided peptides can be flanked by non-alpha Connexin or non-ACT peptide Connexin amino acids. Examples of the flanking non-alpha Connexin and non-ACT Connexin amino acids are provided herein. An example of non-ACT Connexin amino acids are the carboxy-terminal 20 to 120 amino acids of mouse Cx43 (SEQ ID NO:71). Another example of non-ACT Connexin amino acids are the carboxy-terminal 20 to 120 amino acids of human Cx43 (SEQ ID NO: 72). Another example would be the carboxy-terminal 20 to 120 amino acids of chick Cx43 (SEQ ID NO: 73). Another example would be the carboxy-terminal 20 to 120 amino acids of human Cx45 (SEQ ID NO: 74). Another example would be the carboxy-terminal 20 to 120 amino acids of chick Cx45 (SEQ ID NO: 75). Another example would be the carboxy-terminal 20 to 120 amino of human Cx37 (SEQ ID NO: 76). Another example would be the carboxy-terminal 20 to 120 amino acids of rat Cx33 (SEQ ID NO: 77).

An example of a non-alpha Connexin is the 239 amino acid sequence of enhanced green fluorescent protein (SEQ ID NO: 78). In another aspect, given that ACT-1 is shown to be functional when fused to the carboxy terminus of the 239 amino acid sequence of GFP, ACT peptides are expected to retain function when flanked with non-Connexin polypeptides of up to at least 239 amino acids.

In embodiments, the ACT sequence is maintained as the free carboxy terminus of a given polypeptide, and the ACT peptide is able to access its targets. Thus, polypeptides exceeding 239 amino acids in addition to the ACT peptide can affect glioma cell invasiveness through effects on adhesion, motility, and/or invasion, or promoting a bystander effect for chemotherapeutic agents such as temozolomide.

Connexins are the sub-unit protein of the gap junction channel which is responsible for intercellular communication (Goodenough and Paul, 2003). Based on patterns of conservation of nucleotide sequence, the genes encoding Connexin proteins are divided into two families termed the alpha and beta Connexin genes. The carboxy-terminal-most amino acid sequences of alpha Connexins are characterized by multiple distinctive and conserved features (see Table 2). This conservation of organization is consistent with the ability of ACT peptides to form distinctive 3D structures, interact with multiple partnering proteins, mediate interactions with lipids and membranes, interact with nucleic acids including DNA, transit and/or block membrane channels and provide consensus motifs for proteolytic cleavage, protein cross-linking, ADP-ribosylation, glycosylation and phosphorylation. Thus, the provided polypeptide interacts with a domain of a protein that normally mediates the binding of said protein to the carboxy-terminus of an alpha Connexin. For example, nephroblastoma overexpressed protein (NOV) interacts with a Cx43 c-terminal domain (Fu et al., J. Biol. Chem. 2004 279(35):36943-50). It is considered that this and other proteins interact with the carboxy-terminus of alpha Connexins and further interact with other proteins forming a macromolecular complex. Thus, the provided polypeptide can inhibit the operation of a molecular machine, such as, for example, one involved in regulating the aggregation of Cx43 gap junction channels.

As used herein, “inhibit,” “inhibiting,” and “inhibition” mean to decrease an activity, response, condition, disease, or other biological parameter. This can include, but is not limited to, the complete loss of activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The ACT sequence of the provided polypeptide can be from any alpha Connexin. Thus, the alpha Connexin component of the provided polypeptide can be from a human, murine, bovine, monotrene, marsupial, primate, rodent, cetacean, mammalian, avian, reptilian, amphibian, piscine, chordate, protochordate or other alpha Connexin.

Thus, the provided polypeptide can comprise an ACT of a Connexin selected from the group consisting of mouse Connexin 47, human Connexin 47, Human Connexin 46.6, Cow Connexin 46.6, Mouse Connexin 30.2, Rat Connexin 30.2, Human Connexin 31.9, Dog Connexin 31.9, Sheep Connexin 44, Cow Connexin 44, Rat Connexin 33, Mouse Connexin 33, Human Connexin 36, mouse Connexin 36, rat Connexin 36, dog Connexin 36, chick Connexin 36, zebrafish Connexin 36, morone Connexin 35, morone Connexin 35, Cynops Connexin 35, Tetraodon Connexin 36, human Connexin 37, chimp Connexin 37, dog Connexin 37, Cricetulus Connexin 37, Mouse Connexin 37, Mesocricetus Connexin 37, Rat Connexin 37, mouse Connexin 39, rat Connexin 39, human Connexin 40.1, Xenopus Connexin 38, Zebrafish Connexin 39.9, Human Connexin 40, Chimp Connexin 40, dog Connexin 40, cow Connexin 40, mouse Connexin 40, rat Connexin 40, Cricetulus Connexin 40, Chick Connexin 40, human Connexin 43, Cercopithecus Connexin 43, Oryctolagus Connexin 43, Spermophilus Connexin 43, Cricetulus Connexin 43, Phodopus Connexin 43, Rat Connexin 43, Sus Connexin 43, Mesocricetus Connexin 43, Mouse Connexin 43, Cavia Connexin 43, Cow Connexin 43, Erinaceus Connexin 43, Chick Connexin 43, Xenopus Connexin 43, Oryctolagus Connexin 43, Cyprinus Connexin 43, Zebrafish Connexin 43, Danio aequipinnatus Connexin 43, Zebrafish Connexin 43.4, Sheep Connexin 44, Zebrafish Connexin 44.2, Zebrafish Connexin 44.1, human Connexin 45, chimp Connexin 45, dog Connexin 45, mouse Connexin 45, cow Connexin 45, rat Connexin 45, chick Connexin 45, Tetraodon Connexin 45, chick Connexin 45, human Connexin 46, chimp Connexin 46, mouse Connexin 46, dog Connexin 46, rat Connexin 46, Mesocricetus Connexin 46, Cricetulus Connexin 46, human Connexin 46.6, mouse connexin 47, Chick Connexin 56, Zebrafish Connexin 39.9 cow Connexin 49, human Connexin 50, chimp Connexin 50, rat Connexin 50, mouse Connexin 50, dog Connexin 50, sheep Connexin 49, Mesocricetus Connexin 50, Cricetulus Connexin 50, Chick Connexin 50, human Connexin 59, or other alpha Connexin.

Amino acid sequences for alpha connexins are known in the art and include for example those identified in Table 1 by accession number.

TABLE 1
Alpha Connexins

| Protein | Accession No. | Protein | Accession No. |
|---|---|---|---|
| mouse Connexin 47 | NP_536702 | Phodopus Connexin 43 | AAR33085 |
| human Connexin 47 | AAH89439 | Rat Connexin 43 | AAH81842 |
| Human Connexin 46.6 | AAB94511 | Sus Connexin 43 | AAR33087 |
| Cow Connexin 46.6 | XP_582393 | Mesocricetus Connexin | AAO61857 |
| | | 43 | |
| Mouse Connexin 30.2 | NP_848711 | Mouse Connexin 43 | AAH55375 |
| Rat Connexin 30.2 | XP_343966 | Cavia Connexin 43 | AAU06305 |
| Human Connexin 31.9 | AAM18801 | Cow Connexin 43 | NP_776493 |
| Dog Connexin 31.9 | XP_548134 | Erinaceus Connexin 43 | AAR33083 |
| Sheep Connexin 44 | AAD56220 | Chick Connexin 43 | AAA53027 |
| Cow Connexin 44 | I46053 | Xenopus Connexin 43 | NP_988856 |
| Rat Connexin 33 | P28233 | Oryctolagus Connexin | AAS89649 |
| | | 43 | |
| Mouse Connexin 33 | AAR28037 | Cyprinus Connexin 43 | AAG17938 |
| Human Connexin 36 | Q9UKL4 | Zebrafish Connexin 43 | CAH69066 |
| mouse Connexin 36 | NP_034420 | Danio | AAC19098 |
| | | aequipinnatus Connexin | |
| | | 43 | |
| rat Connexin 36 | NP_062154 | Zebrafish Connexin | NP_571144 |
| | | 43.4 | |
| dog Connexin 36 | XP_544602 | Zebrafish Connexin | AAH45279 |
| | | 44.2 | |
| chick Connexin 36 | NP_989913 | Zebrafish Connexin | NP_571884 |
| | | 44.1 | |
| zebrafish Connexin 36 | NP_919401 | human Connexin45 | I38430 |
| morone Connexin 35 | AAC31884 | chimp Connexin45 | XP_511557 |
| morone Connexin 35 | AAC31885 | dog Connexin 45 | XP_548059 |
| Cynops Connexin 35 | BAC22077 | mouse Connexin 45 | AAH71230 |
| Tetraodon Connexin 36 | CAG06428 | cow Connexin 45 | XP_588395 |
| human Connexin 37 | I55593 | rat Connexin 45 | AAN17802 |
| chimp Connexin 37 | XP_524658 | chick Connexin45 | NP_990834 |
| dog Connexin 37 | XP_539602 | Tetraodon Connexin 45 | CAF93782 |
| Cricetulus Connexin 37 | AAR98615 | chick Connexin 45.6 | I50219 |
| Mouse Connexin 37 | AAH56613 | human Connexin 46 | NP_068773 |
| Mesocricetus Connexin | AAS83433 | chimp Connexin 46 | XP_522616 |
| 37 | | | |
| Rat Connexin 37 | AAH86576 | mouse Connexin 46 | NP_058671 |
| mouse Connexin 39 | NP_694726 | dog Connexin 46 | XP_543178 |
| rat Connexin 39 | AAN17801 | rat Connexin 46 | NP_077352 |
| human Connexin 40.1 | NP_699199 | Mesocricetus Connexin | AAS83437 |
| | | 46 | |
| Xenopus Connexin 38 | AAH73347 | Cricetulus Connexin 46 | AAS77618 |
| Zebrafish Connexin | NP_997991 | human Connexin 46.6 | NM_020435.3 |
| 39.9 | | | |
| Human Connexin 40 | NP_859054 | Chick Connexin 56 | A45338 |
| Chimp Connexin 40 | XP_513754 | Zebrafish Connexin | NP_997991 |
| | | 39.9 | |
| dog Connexin 40 | XP_540273 | cow Connexin 49 | XP_602360 |
| cow Connexin 40 | XP_587676 | human Connexin 50 | P48165 |
| mouse Connexin 40 | AAH53054 | chimp Connexin 50 | XP_524857 |
| rat Connexin 40 | AAH70935 | rat Connexin 50 | NP_703195 |
| Cricetulus Connexin 40 | AAP37454 | mouse Connexin 50 | AAG59880 |
| Chick Connexin 40 | NP_990835 | dog Connexin 50 | XP_540274 |
| human Connexin 43 | P17302 | sheep Connexin 49 | AAF01367 |
| Cercopithecus Connexin | AAR33082 | Mesocricetus Connexin | AAS83438 |
| 43 | | 50 | |
| Oryctolagus Connexin | AAR33084 | Cricetulus Connexin 50 | AAR98618 |
| 43 | | | |
| Spermophilus Connexin | AAR33086 | Chick Connexin 50 | BAA05381 |
| 43 | | | |
| Cricetulus Connexin 43 | AAO61858 | human Connexin 59 | AAGF09406 |
| Sheep Connexin 44 | AF177912 | mouse Connexin 47 | AJ276435.1 |

Thus, the provided polypeptide can comprise the amino acid sequence SEQ ID NO:1, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:43, SEQ ID NO:90 or SEQ ID NO:91, and/or conservative variants or fragments thereof.

The 20-30 carboxy-terminal-most amino acid sequence of alpha Connexins are characterized by a distinctive and conserved organization. This distinctive and conserved organization would include a type II PDZ binding motif (φ-x-φ; wherein x=any amino acid and φ=a Hydrophobic amino acid; e.g., Table 2, BOLD) and proximal to this motif, Proline (P) and/or Glycine (G) hinge residues; a high frequency phospho-Serine (S) and/or phospho-Threonine (T) residues; and a high frequency of positively charged Arginine (R), Lysine (K) and negatively charged Aspartic acid (D) or Glutamic acid (E) amino acids. For many alpha Connexins, the P and G residues occur in clustered motifs (e.g., Table 2, italicized) proximal to the carboxy-terminal type II PDZ binding motif. The S and T phosphor-amino acids of most alpha Connexins also are typically organized in clustered, repeat-like motifs (e.g., Table 2, underlined). This organization is particularly the case for Cx43, where 90% of 20 carboxyl terminal-most amino acids are comprised of the latter seven amino acids. In a further example of the high conservation of the sequence, ACT peptide organization of Cx43 is highly conserved from humans to fish (e.g., compare Cx43 ACT sequences for humans and zebrafish in Table 2). In another example, the ACT peptide organization of Cx45 is highly conserved from humans to birds (e.g., compare Cx45 ACT sequences for humans and chick in Table 2). In another example, the ACT peptide organization of Cx36 is highly conserved from primates to fish (e.g., compare Cx36 ACT sequences for chimp and zebrafish in Table 2).

TABLE 2
Alpha Connexin Carboxy-Terminal (ACT) Amino Acid
Sequences

| Gene | Sequence | SEQ ID NO |
|---|---|---|
| Human alpha | P SSRA SSRA SSR PRP D | (SEQ ID NO: 1) |
| Cx43 | DLEI | |
| Chick alpha | P S RA SSRA SSR PRP D | (SEQ ID NO: 29) |
| Cx43 | DLEI | |
| Zebrafish | P CSRA SSRM SSRA R P D | (SEQ ID NO: 90) |
| alpha | DLDV | |
| Cx43 | | |
| Human alpha | G SNKS TA SSKS GDG KN | (SEQ ID NO: 30) |
| Cx45 | SVWI | |
| Chick alpha | G SNKSS A SSKS GDG KN | (SEQ ID NO: 31) |
| Cx45 | SVWI | |
| Human alpha | G RA SKAS RASS GRARP E | (SEQ ID NO: 32) |
| Cx46 | DLAI | |
| Human alpha | G SASS RD G K TVWI | (SEQ ID NO: 33) |
| Cx46.6 | | |
| Chimp alpha | P RVSV PNFG R TQ SSD | (SEQ ID NO: 34) |
| Cx36 | SAYV | |
| Chick alpha | P RMSM PNFG R TQ SSD | (SEQ ID NO: 35) |
| Cx36 | SAYV | |
| Zebrafish | P RMSM PNFG R TQ SSD S | (SEQ ID NO: 91) |
| alpha | AYV | |
| Cx36 | | |
| Human alpha | P RAGSEK G SASS R DG KT | (SEQ ID NO: 36) |
| Cx47 | TVWI | |
| Human alpha | G HRL [symbol] YHSDKRRL | (SEQ ID NO: 37) |
| Cx40 | SKASS KARSD DLSV | |
| Human alpha | P ELTTDDAR P LSRL SKASS | (SEQ ID NO: 38) |
| Cx50 | RARSD DLTV | |
| Human alpha | P NHVV SLTN NLI GRRVP T | (SEQ ID NO: 39) |
| Cx59 | DLQI | |
| Rat alpha | P S CV SSS A VLTTIC SS | (SEQ ID NO: 40) |
| Cx33 | DQVV PVG L SS FYM | |
| Sheep alpha | G R SSKA SKSS GG RARAA | (SEQ ID NO: 41) |
| Cx44 | DLAI | |
| Human beta | LC YLLIR YCSGK SKKPV | (SEQ ID NO: 42) |
| Cx26 | | |

Thus, in one aspect, the provided polypeptide comprises one, two, three or all of the amino acid motifs selected from the group consisting of 1) a type II PDZ binding motif, 2) Proline (P) and/or Glycine (G) hinge residues; 3) clusters of phospho-Serine (S) and/or phospho-Threonine (T) residues; and 4) a high frequency of positively charged Arginine (R) and Lysine (K) and negatively charged Aspartic acid (D) and/or Glutamic acid (E) amino acids). In another aspect, the provided polypeptide comprises a type II PDZ binding motif at the carboxy-terminus, Proline (P) and/or Glycine (G) hinge residues proximal to the PDZ binding motif, and positively charged residues (K, R, D, E) proximal to the hinge residues.

PDZ domains were originally identified as conserved sequence elements within the postsynaptic density protein PSD95/SAP90, the Drosophila tumor suppressor dlg-A, and the tight junction protein ZO-1. Although originally referred to as GLGF or DHR motifs, they are now known by an acronym representing these first three PDZ-containing proteins (PSD95/DLG/ZO-1). These 80-90 amino acid sequences have now been identified in well over 75 proteins and are characteristically expressed in multiple copies within a single protein. Thus, in one aspect, the provided polypeptide can inhibit the binding of an alpha Connexin to a protein comprising a PDZ domain. The PDZ domain is a specific type of protein-interaction module that has a structurally well-defined interaction ‘pocket’ that can be filled by a PDZ-binding motif, referred to herein as a “PDZ motif”. PDZ motifs are consensus sequences that are normally, but not always, located at the extreme intracellular carboxyl terminus. Four types of PDZ motifs have been classified: type I (S/T-x-φ), type II (φ-x-φ), type III (ψ-x-φ) and type IV (D-x-V), where x is any amino acid, φ is a hydrophobic residue (V, I, L, A, G, W, C, M, F) and ψ is a basic, hydrophilic residue (H, R, K). (Songyang, Z., et al. 1997. Science 275, 73-77). In one aspect, the provided polypeptide comprises a type II PDZ binding motif.

It is noted that the 18 carboxy-terminal-most amino acid sequence of alpha Cx37 represents an exceptional variation on the ACT peptide theme. The Cx37 ACT-like sequence is GQKPPSRPSSSASKKQ*YV (SEQ ID NO: 43). Thus the carboxy terminal 4 amino acids of Cx37 conform only in part to a type II PDZ binding domain. Instead of a classical type II PDZ binding domain, Cx37 has a neutral Q* at position 2 where a hydrophobic amino acid would be expected. As such Cx37 comprises what might be termed a type II PDZ binding domain-like sequence. Nonetheless, Cx37 strictly maintains all other aspects of ACT peptide organization including clustered serine residues, frequent R and K residues and a P-rich sequence proximal to the PDZ binding domain-like sequence. Given this overall level of conservation of ACT-like organization in common with the other >70 alpha Connexins listed above, it is understood that the Cx37 ACT-like carboxy terminus functions in the provided capacity.

For comparison, the beta Connexin Cx26 is shown in Table 2. Cx26 has no carboxyl terminal type II PDZ binding motif; less than 30% of the carboxyl terminal most amino acids comprise S, T, R, D or E residues; it has no evidence of motifs proximal to a type II PDZ binding motif or PDZ binding like motif containing clusters of P and G hinge residues; and no evidence of clustered, repeat-like motifs of serine and threonine phospho-amino acids. Cx26 does have three Lysine (K) residues, clustered one after the other near the carboxy terminus of the sequence. However, no alpha Connexin surveyed in the >70 alpha Connexins listed above was found to display this feature of three repeated K residues domain at carboxy terminus (Cx26 is a beta connexin, thus by definition does not have an ACT domain).

As provided herein, the unique functional characteristics of this relatively short stretch of amino acids encompass unexpected roles in altering glioma cell invasiveness through increased adhesion, decreased motility, and decreased invasion or promoting a bystander effect when administered with a chemotherapeutic agent. Thus, in one aspect, the provided polypeptide comprises a type II PDZ binding motif (φ-x-φ; wherein x=any amino acid and φ=a Hydrophobic amino acid). In another aspect, greater than 50%, 60%, 70%, 80%, 90% of the amino acids of the provided ACT polypeptide is comprised one or more of Proline (P), Glycine (G), phospho-Serine (S), phospho-Threonine (T), Arginine (R), Lysine (K), Aspartic acid (D), or Glutamic acid (E) amino acid residues.

The amino acids Proline (P), Glycine (G), Arginine (R), Lysine (K), Aspartic acid (D), and Glutamic acid (E) are necessary determinants of protein structure and function. Proline and Glycine residues provide for tight turns in the 3D structure of proteins, enabling the generation of folded conformations of the polypeptide required for function. Charged amino acid sequences are often located at the surface of folded proteins and are necessary for chemical interactions mediated by the polypeptide including protein-protein interactions, protein-lipid interactions, enzyme-substrate interactions and protein-nucleic acid interactions. Thus, in another aspect Proline (P) and Glycine (G) Lysine (K), Aspartic acid (D), and Glutamic acid (E) rich regions proximal to the type II PDZ binding motif provide for properties necessary to the provided actions of ACT peptides. In another aspect, the provided polypeptide comprises Proline (P) and Glycine (G) Lysine (K), Aspartic acid (D), and/or Glutamic acid (E) rich regions proximal to the type II PDZ binding motif.

Phosphorylation is the most common post-translational modification of proteins and is crucial for modulating or modifying protein structure and function. Aspects of protein structure and function modified by phosphorylation include protein conformation, protein-protein interactions, protein-lipid interactions, protein-nucleic acid interactions, channel gating, protein trafficking and protein turnover. Thus, in one aspect the phospho-Serine (S) and/or phospho-Threonine (T) rich sequences are necessary for modifying the function of ACT peptides, increasing or decreasing efficacy of the polypeptides in their provided actions. In another aspect, the provided polypeptide comprises Serine (S) and/or phospho-Threonine (T) rich sequences or motifs.

In another example, respecting definition of an ACT peptide, it is highly auspicious, in light of the high degree of tissue/organ regeneration potential in lower animals such as fish, that a methionine occurs near the amino terminus of the ACT sequence of zebrafish Cx43 (Table 2). In addition to encoding methionine, the methionine base pair triplet is an alternate translation start site. If translation initiated from this methionine, the sequence SSRARPDDLDV (SEQ ID NO:90), would be produced. This translation product maintains all the conserved and distinctive features of a canonical ACT peptide. Specifically this peptide comprises a carboxy terminal type II PDZ binding domain and has a domain enriched in P, R and D residues proximal to the PDZ binding domain. In addition, the sequence comprises a clustered S motif, with potential to modulate ACT peptide function at its amino terminal. This raises the interesting prospect that animals with high tissue/organ regeneration potential such as fish may translate ACT peptides sequences directly.

Thus, the provided polypeptide can comprise the c-terminal sequence of human Cx43. Thus, the provided polypeptide can comprise the amino acid sequence SEQ ID NO:1 or SEQ ID NO:2. The polypeptide can comprise 9 amino acids of the carboxy terminus of human Cx40. Thus, the polypeptide can comprise the amino acid sequence SEQ ID NO:5.

When specific proteins are referred to herein, variants, derivatives, and fragments are contemplated. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known and include, for example, M13 primer mutagenesis and PCR mutagenesis. Amino acid substitutions are typically of single residues, but can occur at a number of different locations at once; insertions usually will be on the order of about from 1 to 10 amino acid residues. Deletions or insertions preferably are made in adjacent pairs, i.e., a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure unless such a change in secondary structure of the mRNA is desired. Substitutional variants are those in which at least one residue has been removed and a different residue inserted in its place. Such substitutions generally are made in accordance with the following Table 3 and are referred to as conservative substitutions.

TABLE 3
Amino acid substitutions

| Original Residue | Exemplary Substitutions |
|---|---|
| Ala | Ser |
| Arg | Lys |
| Asn | Gln |
| Asp | Glu |
| Cys | Ser |
| Gln | Asn |
| Glu | Asp |
| Gly | Pro |
| His | Gln |
| Ile | Leu; Val |
| Leu | Ile; Val |
| Lys | Arg; Gln |
| Met | Leu; Ile |
| Phe | Met; Leu; Tyr |
| Pro | Gly |
| Ser | Thr |
| Thr | Ser |
| Trp | Tyr |
| Tyr | Trp; Phe |
| Val | Ile; Leu |

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations shown in Table 3. Conservatively substituted variations of each explicitly disclosed sequence are included within the polypeptides provided herein.

Typically, conservative substitutions have little to no impact on the biological activity of a resulting polypeptide. In a particular example, a conservative substitution is an amino acid substitution in a peptide that does not substantially affect the biological function of the peptide. A peptide can include one or more amino acid substitutions, e.g., 2-10 conservative substitutions, 2-5 conservative substitutions, 4-9 conservative substitutions, such as 2, 5 or 10 conservative substitutions.

A polypeptide can be produced to contain one or more conservative substitutions by manipulating the nucleotide sequence that encodes that polypeptide using, for example, standard procedures such as site-directed mutagenesis or PCR. Alternatively, a polypeptide can be produced to contain one or more conservative substitutions by using standard peptide synthesis methods. An alanine scan can be used to identify which amino acid residues in a protein can tolerate an amino acid substitution. In one example, the biological activity of the protein is not decreased by more than 25%, for example not more than 20%, for example not more than 10%, when an alanine, or other conservative amino acid (such as those listed below), is substituted for one or more native amino acids.

Further information about conservative substitutions can be found in, among other locations, in Ben-Bassat et al., (J. Bacteriol. 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988) and in standard textbooks of genetics and molecular biology.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W.H. Freeman & Co., San Francisco pp 79-86 [1983]), acetylation of the N-terminal amine and, in some instances, amidation of the C-terminal carboxyl.

It is understood that there are numerous amino acid and peptide analogs which can be incorporated into the disclosed compositions. For example, there are numerous D amino acids or amino acids which have a different functional substituent than the amino acids shown in Table 3. The opposite stereoisomers of naturally occurring peptides are disclosed, as well as the stereoisomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994), all of which are herein incorporated by reference at least for material related to amino acid analogs).

Molecules can be produced that resemble polypeptides, but which are not connected via a natural peptide linkage. For example, linkages for amino acids or amino acid analogs can include CH2NH—, —CH2S—, —CH2-CH2-, —CH═CH— (cis and trans), —COCH2-, —CH(OH)CH2-, and —CHH2SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH2NH—, CH2CH2-); Spatola et al. Life Sci 38:1243-1249 (1986) (—CH H2-S); Hann J. Chem. Soc Perkin Trans. I 307-314 (1982) (—CH—CH—, cis and trans); Almquist et al. J. Med. Chem. 23:1392-1398 (1980) (—COCH2-); Jennings-White et al. Tetrahedron Lett 23:2533 (1982) (—COCH2-); Szelke et al. European App., EP 45665 CA (1982): 97:39405 (1982) (—CH(OH)CH2-); Holladay et al. Tetrahedron. Lett 24:4401-4404 (1983) (—C(OH)CH2-); and Hruby Life Sci 31:189-199 (1982) (—CH2-S—); each of which is incorporated herein by reference. It is understood that peptide analogs can have more than one atom between the bond atoms, such as b-alanine, g-aminobutyric acid, and the like.

Amino acid analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, greater ability to cross biological barriers (e.g., gut, blood vessels, blood-brain-barrier), and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

Thus, the provided polypeptide can comprise a conservative variant of the c-terminus of an alpha Connexin (ACT). As shown in Table 4, an example of a single conservative substitution within the sequence SEQ ID NO:2 is given in the sequence SEQ ID NO:3. An example of three conservative substitutions within the sequence SEQ ID NO:2 is given in the sequence SEQ ID NO:4. Thus, the provided polypeptide can comprise the amino acid SEQ ID NO:3 or SEQ ID NO:4.

TABLE 4
ACT Polypeptides Variants Sequence

| Sequence | SEQ ID NO |
|---|---|
| RPRPDDLEI | SEQ ID NO: 2 |
| RPRPDDLEV | SEQ ID NO: 3 |
| RPRPDDVPV | SEQ ID NO: 4 |
| SSRASSRASSRPRPDDLEV | SEQ ID NO: 44 |
| RPKPDDLEI | SEQ ID NO: 45 |
| SSRASSRASSRPKPDDLEI | SEQ ID NO: 46 |
| RPKPDDLDI | SEQ ID NO: 47 |
| SSRASSRASSRPRPDDLDI | SEQ ID NO: 48 |
| SSRASTRASSRPRPDDLEI | SEQ ID NO: 49 |
| RPRPEDLEI | SEQ ID NO: 50 |
| SSRASSRASSRPRPEDLEI | SEQ ID NO: 51 |
| GDGKNSVWV | SEQ ID NO: 52 |
| SKAGSNKSTASSKSGDGKNSVWV | SEQ ID NO: 53 |
| GQKPPSRPSSSASKKLYV | SEQ ID NO: 54 |
| RPRPDDELI | SEQ ID NO: 92 |

It is understood that one way to define any variants, modifications, or derivatives of the disclosed genes and proteins herein is through defining the variants, modification, and derivatives in terms of sequence identity (also referred to herein as homology) to specific known sequences. Specifically disclosed are variants of the nucleic acids and polypeptides herein disclosed which have at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to the stated or known sequence. Those of skill in the art readily understand how to determine the sequence identity of two proteins or nucleic acids. For example, the sequence identity can be calculated after aligning the two sequences so that the sequence identity is at its highest level.

Another way of calculating sequence identity can be performed by published algorithms. Optimal alignment of sequences for comparison may be conducted by the local sequence identity algorithm of Smith and Waterman Adv. Appl. Math. 2: 482 (1981), by the sequence identity alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection. These references are incorporated herein by reference in their entirety for the methods of calculating sequence identity. The same types of sequence identity can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

Thus, the provided polypeptide can comprise an amino acid sequence with at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to the c-terminus of an alpha Connexin (ACT). Thus, in one aspect, the provided polypeptide comprises an amino acid sequence with at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to SEQ ID NO:1, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO: 90 or SEQ ID NO:91. As an example, provided is a polypeptide (SEQ ID NO:4) having 66% sequence identity to the same stretch of 9 amino acids occurring on the carboxy-terminus of human Cx43 (SEQ ID NO:2).

The herein provided polypeptides can be added directly to a glioma in a subject. However, efficiency of cytoplasmic localization of the provided polypeptide is enhanced by cellular internalization transporter chemically linked in cis or trans with the polypeptide. Efficiency of cell internalization transporters are enhanced further by light or co-transduction of cells with Tat-HA peptide.

Thus, the provided polypeptide can comprise a cellular internalization transporter or sequence. The cellular internalization sequence can be any internalization sequence known or newly discovered in the art, or conservative variants thereof. Non-limiting examples of cellular internalization transporters and sequences include Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynBI, Pep-7, HN-1, BGSC (Bis-Guanidinium-Spermidine-Cholesterol, and BGTC (Bis-Guanidinium-Tren-Cholesterol) (see Table 5).

TABLE 5
Cell Internalization Transporters

| Name | Sequence | SEQ ID NO |
|---|---|---|
| Antp | RQPKIWFPNRRKPWKK | (SEQ ID NO: 7) |
| HIV-Tat | GRKKRRQRPPQ | (SEQ ID NO: 14) |
| Penetratin | RQIKIWFQNRRMKWKK | (SEQ ID NO: 15) |
| Antp-3A | RQIAIWFQNRR MKWAA | (SEQ ID NO: 16) |
| Tat | RKKRRQRRR | (SEQ ID NO: 17) |
| Buforin II | TRSSRAGLQFPVGRVHRLLRK | (SEQ ID NO: 18) |
| Transportan | GWTLNSAGYLLGKINKALAALAKKIL | (SEQ ID NO: 19) |
| model amphipathic | KLALKLALKALKAALKLA | (SEQ ID NO: 20) |
| peptide (MAP) | | |
| K-FGF | AAVALLPAVLLALLAP | (SEQ ID NO: 21) |
| Ku70 | VPMLK-PMLKE | (SEQ ID NO: 22) |
| Prion | MANLGYWLLALFVTMWTDVGLCKKRPKP | (SEQ ID NO: 23) |
| pVEC | LLIILRRRIRKQAHAHSK | (SEQ ID NO: 24) |
| Pep-1 | KETWWETWWTEWSQPKKKRKV | (SEQ ID NO: 25) |
| SynB1 | RGGRLSYSRRRFSTSTGR | (SEQ ID NO: 26) |
| Pep-7 | SDLWEMMMVSLACQY | (SEQ ID NO: 27) |
| HN-1 | TSPLNIHNGQKL | (SEQ ID NO: 28) |

BGSC (Bis-Guanidinium-Spermidine-Cholesterol) 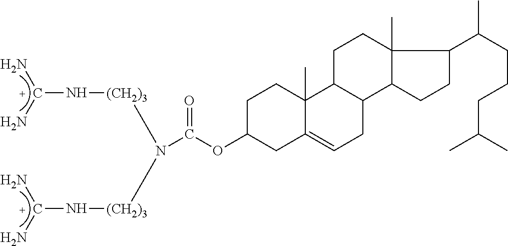 BGSC
BGTC (Bis-Guanidinium-Tren-Cholesterol) 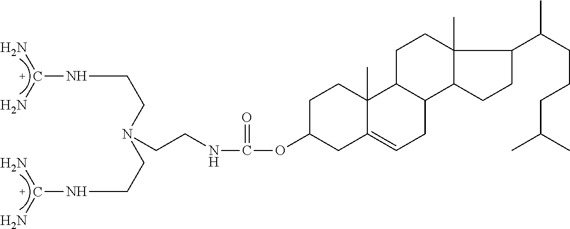 [SMILES: CC(C)CCCC(C)C1CCC2C3CC=C4CC(OC(=O)NCCN(CCNC(N)N)CCNC(N)N)CCC4(C)C3CCC12C] BGTC

Thus, the provided polypeptide can further comprise the amino acid sequence SEQ ID NO:7, SEQ ID NO:14 (Bucci, M. et al. 2000. Nat. Med. 6, 1362-1367), SEQ ID NO:15 (Derossi, D., et al. 1994. Biol. Chem. 269, 10444-10450), SEQ ID NO:16 (Fischer, P. M. et al 2000. J. Pept. Res. 55, 163-172), SEQ ID NO:17 (Frankel, A. D. & Pabo, C. O. 1988. Cell 55, 1189-1193; Green, M. & Loewenstein, P. M. 1988. Cell 55, 1179-1188), SEQ ID NO:18 (Park, C. B., et al. 2000. Proc. Natl. Acad. Sci. USA 97, 8245-8250), SEQ ID NO:19 (Pooga, M., et al. 1998. FASEB J. 12, 67-77), SEQ ID NO:20 (Oehlke, J. et al. 1998. Biochim. Biophys. Acta. 1414, 127-139), SEQ ID NO:21 (Lin, Y. Z., et al. 1995. J. Biol. Chem. 270, 14255-14258), SEQ ID NO:22 (Sawada, M., et al. 2003. Nature Cell Biol. 5, 352-357), SEQ ID NO:23 (Lundberg, P. et al. 2002. Biochem. Biophys. Res. Commun. 299, 85-90), SEQ ID NO:24 (Elmquist, A., et al. 2001. Exp. Cell Res. 269, 237-244), SEQ ID NO:25 (Morris, M. C., et al. 2001. Nature Biotechnol. 19, 1173-1176), SEQ ID NO:26 (Rousselle, C. et al. 2000. Mol. Pharmacol. 57, 679-686), SEQ ID NO:27 (Gao, C. et al. 2002. Bioorg. Med. Chem. 10, 4057-4065), or SEQ ID NO:28 (Hong, F. D. & Clayman, G. L. 2000. Cancer Res. 60, 6551-6556). The provided polypeptide can further comprise BGSC (Bis-Guanidinium-Spermidine-Cholesterol) or BGTC (Bis-Guanidinium-Tren-Cholesterol) (Vigneron, J. P. et al. 1998. Proc. Natl. Acad. Sci. USA. 93, 9682-9686). The preceding references are hereby incorporated herein by reference in their entirety for the teachings of cellular internalization vectors and sequences. Any other internalization sequences now known or later identified can be combined with a peptide of the invention.

The provided polypeptide can comprise any ACT sequence (e.g, any of the ACT peptides disclosed herein) in combination with any of the herein provided cell internalization sequences. Examples of said combinations are given in Table 6. Thus, the provided polypeptide can comprise an Antennapedia sequence comprising amino acid sequence SEQ ID NO:7. Thus, the provided polypeptide can comprise the amino acid sequence SEQ ID NO:8, SEQ ID NO:9, SEQ ID NO:10, SEQ ID NO:11, or SEQ ID NO: 12. In some embodiments, the provided polypeptide may be have at least 65%, 70%, 75%, 80%, 85%, 90% sequence identity to SEQ ID NO:8, SEQ ID NO:9, SEQ ID NO:10, SEQ ID NO:11, or SEQ ID NO: 12.

TABLE 6
ACT Polypeptides with Cell Internalization
Sequences (CIS)

| CIS/ACT | Sequence | SEQ ID NO |
|---|---|---|
| Antp/ACT 2 | RQPKIWFPNRRKPWKK | SEQ ID NO: 8 |
| | PSSRASSRASSRPRPDDLEI | |
| Antp/ACT 1 | RQPKIWFPNRRKPWKK | SEQ ID NO: 9 |
| | RPRPDDLEI | |
| Antp/ACT 3 | RQPKIWFPNRRKPWKK | SEQ ID NO: 10 |
| | RPRPDDLEV | |
| Antp/ACT 4 | RQPKIWFPNRRKPWKK | SEQ ID NO: 11 |
| | RPRPDDVPV | |
| Antp/ACT 5 | RQPKIVVFPNRRKPWKK | SEQ ID NO: 12 |
| | KARSDDLSV | |
| HIV-Tat/ACT 1 | GRKRRQRPPQ | SEQ ID NO: 56 |
| | RPRPDDLEI | |
| Penetratin/ | RQIKIWFQNRRMKWKK | SEQ ID NO: 57 |
| ACT 1 | RPRPDDLEI | |
| Antp-3A/ACT 1 | RQIAIWFQNRRMKWAA | SEQ ID NO: 58 |
| | RPRPDDLEI | |
| Tat/ACT-1 | RKKRRQRRR RPRPDDLEI | SEQ ID NO: 59 |
| Buforin II/ | TRSSRAGLQFPVGRVHRLLRK | SEQ ID NO: 60 |
| ACT 1 | RPRPDDLEI | |
| Transportan/ | GWTLNSAGYLLGKINKALAAL | SEQ ID NO: 61 |
| ACT 1 | AKKIL RPRPDDLEI | |
| MAP/ACT 1 | KLALKLALKALKAALKLA | SEQ ID NO: 62 |
| | RPRPDDLEI | |
| K-FGF/ACT 1 | AAVALLPAVLLALLAP | SEQ ID NO: 63 |
| | RPRPDDLEI | |
| Ku70/ACT 1 | VPMLKPMLKE RPRPDDLEI | SEQ ID NO: 64 |
| Prion/ACT I | MANLGYWLLALFVTIVIWTDVG | SEQ ID NO: 65 |
| | LCKKRPKPR PRPDDLEI | |
| pVEC/ACT 1 | LLIILRRRIRKQAHAHSK | SEQ ID NO: 66 |
| | RPRPDDLEI | |
| Pep-1/ACT 1 | KETWWETWWTEWSQPKKKR | SEQ ID NO: 67 |
| | KV RPRPDDLEI | |
| SynB1/ACT 1 | RGGRLSYSRRRFSTSTGR | SEQ ID NO: 68 |
| | RPRPDDLEI | |
| Pep-7/ACT 1 | SDLWEMMMVSLACQY | SEQ ID NO: 69 |
| | RPRPDDLEI | |
| HN-1/ACT 1 | TSPLNIHNGQKL | SEQ ID NO: 70 |
| | RPRPDDLEI | |

Also provided are nucleic acids encoding the polypeptides provided herein. The disclosed nucleic acids are made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U.

By “isolated” or “purified” nucleic acid, polypeptide, or peptide is meant DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (e.g., a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated” or “purified” nucleic acid also refers to RNA, e.g., an mRNA molecule that is encoded by an isolated DNA molecule, or that is chemically synthesized, or that is separated or substantially free from at least some cellular components, e.g., other types of RNA molecules or polypeptide molecules. In the context of this disclosure, when referring to a nucleic acid, polypeptide, or peptide, for example, this is understood to also include an isolated version thereof.

Thus, provided is a nucleic acid encoding a polypeptide comprising the amino acid sequence SEQ ID NO:1, SEQ ID NO:2, SEQ ID NO:3, SEQ ID NO:4, SEQ ID NO:5, SEQ ID NO:7, SEQ ID NO:8, SEQ ID NO:9, SEQ ID NO:10, SEQ ID NO:11, or SEQ ID NO:12.

Thus, the provided nucleic acid can comprise the nucleic acid sequence SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87 SEQ ID NO:88, or SEQ ID NO:89.

The herein provided nucleic acid can be operably linked to an expression control sequence. Also provided is a vector comprising one or more of the herein provided nucleic acids, wherein the nucleic acid is operably linked to an expression control sequence. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as SEQ ID NO:6, into the cell without degradation and include a promoter yielding expression of the gene in the cells into which it is delivered. In some embodiments the promoters are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also disclosed are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. Also disclosed is a viral vector which has been engineered so as to suppress the immune response of the host organism, elicited by the viral antigens. Vectors of this type can carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be based on an adenovirus which has had the E1 gene removed, and these virons are generated in a cell line such as the human 293 cell line. In one aspect, both the E1 and E3 genes are removed from the adenovirus genome.

Another type of viral vector is based on an adeno-associated virus (AAV). This defective parvovirus can infect many cell types and is nonpathogenic to humans. AAV type vectors can transport about 4 to 5 kb and wild type AAV is known to stably insert into chromosome 19. As an example, this vector can be the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In a type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B 19 parvovirus.

Typically the AAV and B 19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Molecular genetic experiments with large human herpes viruses have provided a means whereby large heterologous DNA fragments can be cloned, propagated and established in cells permissive for infection with herpes viruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable. Maintenance of these episomes requires a specific EBV nuclear protein, EBNA1, constitutively expressed during infection with EBV. These vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed polypeptides, nucleic acids, vectors, and/or host cells, for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Feigner et al. Proc. Natl. Acad. Sci. USA 84:7413-7417 (1987); U.S. Pat. No. 4,897,355. Further, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

Nucleic acids that are delivered to cells which are to be integrated into the host cell genome, typically contain integration sequences. These sequences are often viral related sequences, particularly when viral based systems are used. These viral integration systems can be incorporated into nucleic acids which are to be delivered using a non-nucleic acid based system of delivery, such as a liposome, so that the nucleic acid in the delivery system can become integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

Compositions can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Promoters controlling transcription from vectors in mammalian host cells may be obtained from various sources, for example, the genomes of viruses such as: polyoma, Simian Virus 40 (SV40), adenovirus, retroviruses, hepatitis-B virus, cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiefs et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, a-fetoprotein and insulin), typically one will use an enhancer from a eukaryotic cell virus for general expression. Examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A promoter of this type is the CMV promoter (650 bases). Other such promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. The transcription unit can also contain a polyadenylation region. One benefit of this region is that it increases the likelihood that the transcribed unit will be processed and transported like mRNA. The identification and use of polyadenylation signals in expression constructs is well established. Homologous polyadenylation signals can be used in the transgene constructs. In certain transcription units, the polyadenylation region is derived from the SV40 early polyadenylation signal and consists of about 400 bases. Transcribed units can contain other standard sequences alone or in combination with the above sequences improve expression from, or stability of, the construct.

The viral vectors can include nucleic acid sequence encoding a marker product. This marker product is used to determine if the gene has been delivered to the cell and once delivered is being expressed. Example marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: Chinese hamster ovary (CHO) DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1:327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

The nucleic acids may alternatively be a form of therapeutic DNA or RNA molecule designed to inhibit expression of an alpha-connexin in vivo (such as in a glioma cell). The nucleic acids may include, without limitation, antisense oligonucleotides, small interfering RNAs (siRNAs), ribozymes, RNA external guide sequences, short hairpin RNAs (shRNAs), aptamers, or microRNAs (miRNAs). In one embodiment, the nucleic acids inhibit expression of connexin 43. In other embodiments, the nucleic acids may be designed to inhibit expression of any connexin recited herein

As used herein, “antisense oligonucleotides” refer to short (e.g. 13-25 nucleotides) single-stranded, unmodified or chemically modified polynucleotides that are designed to be complementary to a specific sense sequence of a molecule of mRNA, and bind to RNA through Watson-Crick hybridization thus inhibiting its expression. “Small interfering RNA” (“siRNA”) (also referred to in the art as “short interfering RNAs”) refers to a therapeutic RNA, preferably a double-stranded agent, of about 10-50 nucleotides in length (the term “nucleotides” including nucleotide analogs), preferably between about 15-25 nucleotides in length, more preferably about 17, 18, 19, 20, 21, 22, 23, 24, or 25 nucleotides in length, the strands optionally having overhanging ends comprising, for example, 1, 2 or 3 overhanging nucleotides (or nucleotide analogs), which is capable of directing or mediating RNA interference. Naturally-occurring siRNAs are sometimes generated from longer dsRNA molecules (e.g., >25 nucleotides in length) by RNase Dicer. “shRNA”, as used herein, refers to a therapeutic RNA having a stem-loop structure, comprising a first and second region of complementary sequence, the degree of complementarity and orientation of the regions being sufficient such that base pairing occurs between the regions, the first and second regions being joined by a loop region, the loop optionally resulting from a lack of base pairing between nucleotides (or nucleotide analogs) within the loop region. “RNA interference” or “RNAi”, as used herein, refers generally to a sequence-specific or selective process by which a target molecule (e.g., a target gene, protein or RNA) is modulated. In specific embodiments, the process of “RNA interference” or “RNAi” features degradation of RNA molecules, e.g., RNA molecules within a cell, said degradation being triggered by an RNA agent. Degradation is catalyzed by an enzymatic, RNA-induced silencing complex (RISC). RNAi occurs in cells naturally to remove foreign RNAs (e.g., viral RNAs). Natural RNAi is known to proceed via fragments cleaved from free dsRNA which direct the degradative mechanism to other similar RNA sequences. Alternatively, RNAi can be initiated artificially, for example, to modulate or silence the expression of target genes. A “ribozyme” is a molecule of RNA that functions as an enzyme, such as by catalyzing the cleavage of other RNA molecules. Ribozymes may include naturally occurring ribozymes such as RNase P, hairpin ribozyme or hammerhead ribozyme, or artificial ribozymes produced in the laboratory. An “aptamer” is a single-stranded DNA or RNA (ssDNA or ssRNA) molecule or a peptide molecule that can bind to pre-selected targets including proteins and peptides with high affinity and specificity. Aptamers may bind to a target molecules by assuming a variety of shapes due to their propensity to form helices and single-stranded loops. An “RNA external guide sequence” is an RNA molecule derived from a natural tRNA which binds to a target mRNA and renders the mRNA susceptible to hydrolysis by RNase P. “MicroRNA” (miRNAs) are a class of small noncoding RNAs that control gene expression by targeting mRNAs and triggering either translation repression or RNA degradation.

Another embodiment of this disclosure provides polypeptides in the form of antibodies or antibody fragments. In various embodiments, monoclonal or polyclonal antibodies specific to an alpha-Connexin carboxy terminal region can be used in immunoassays to measure the amount of alpha-Connexin or used in immunoaffinity purification of an alpha-Connexin protein. A Hopp & Woods hydrophilic analysis (see Hopp & Woods, Proc. Natl. Acad. Sci. U.S.A. 78:3824-3828 (1981) can be used to identify hydrophilic regions of a protein, and to identify potential epitopes of alpha-Connexin. Further, monoclonal or polyclonal antibodies or antibody fragments generated to be specific to an alpha-Connexin carboxy terminal region may be used in therapeutic methods, such as methods of treating glioma.

The antibodies that immunospecifically bind to an alpha-Connexin carboxy terminal region can be produced by any method known in the art for the synthesis of antibodies, in particular, by chemical synthesis or preferably, by recombinant expression techniques. (See, e.g., U.S. Publication No. 2005/0084449, which is incorporated herein in its entirety). Polyclonal antibodies immunospecific for an alpha-Connexin carboxy terminal region can be produced by various procedures well-known in the art. For example, an alpha-Connexin carboxy terminal region can be administered to various host animals, including, but not limited to, rabbits, mice, and rats, to induce the production of sera containing polyclonal antibodies specific for an alpha-Connexin carboxy terminal region. Various adjuvants may be used to increase the immunological response, depending on the host species, including but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and corynebacterium parvum. Such adjuvants are also well known in the art.

Monoclonal antibodies can be prepared using a wide variety of techniques known in the art, including the use of hybridoma, recombinant, and phage display technologies, or a combination thereof. For example, monoclonal antibodies can be produced using hybridoma techniques, including those known in the art and taught, for example, in Harlow et al., Antibodies: A Laboratory Manual, (Cold Spring Harbor Laboratory Press, 2nd ed. 1988); and Hammerling et al., in: Monoclonal Antibodies and T Cell Hybridomas 563 681 (Elsevier, N. Y., 1981). The term “monoclonal antibody” as used herein is not limited to antibodies produced through hybridoma technology and can refer to an antibody that is derived from a single clone, including any eukaryotic, prokaryotic, or phage clone, and not the method by which it is produced.

Method for producing and screening for specific antibodies using hybridoma technology are routine and well known in the art. Briefly, mice can be immunized with a non-murine antigen, and once an immune response is detected, e.g., antibodies specific for the antigen are detected in the mouse serum, the mouse spleen is harvested and splenocytes isolated. The splenocytes are then fused by well-known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of this disclosure. Ascites fluid, which generally contains high levels of antibodies, can be generated by immunizing mice with positive hybridoma clones.

This disclosure provides methods of generating monoclonal antibodies as well as antibodies produced by the method comprising culturing a hybridoma cell secreting an antibody of the disclosure wherein, preferably, the hybridoma is generated by fusing splenocytes isolated from a mouse immunized with a non-murine antigen with myeloma cells and then screening the hybridomas resulting from the fusion for hybridoma clones that secrete an antibody able to bind to the antigen.

Antibody fragments which recognize specific particular epitopes may be generated by any technique known to those of skill in the art. For example, Fab and F(ab′)2 fragments of the disclosure may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present disclosure can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli, and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present disclosure include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; International application No. PCT/GB91/O1 134; International publication Nos. WO 90/02809; WO 91/10737; WO 92/01047; WO 92/18619; WO 93/11236; WO 95/15982; WO 95/20401; and WO97/13844; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108, which references are each incorporated by reference herein in their entireties.

As described in the above references, after phage selection, the antibody coding regions from the phage can be isolated and used to generate whole antibodies or any other desired antigen binding fragment, and expressed in any desired host, including mammalian cells, insect cells, plant cells, yeast, and bacteria, e.g., as described below. Techniques to produce Fab, Fab′ and F(ab′)2 fragments recombinantly can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise an EF-1α promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, IgG, using techniques known to those of skill in the art.

For some uses, including in vivo use of antibodies in humans and in vitro detection assays, it may be preferable to use humanized antibodies or chimeric antibodies. Human antibodies can be made by a variety of methods known in the art including phage display methods described above using antibody libraries derived from human immunoglobulin sequences. See also U.S. Pat. Nos. 4,444,887 and 4,716,111; and International publication Nos. WO 98/46645, WO 98/50433, WO 98/24893, WO98/16654, WO 96/34096, WO 96/33735, and WO 91/10741.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715; 4,816,567; 4,816,397; and 6,311,415.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains (Fab, Fab′, F(ab′)2, Fabc, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fe), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG1. Where such cytotoxic activity is not desirable, the constant domain may be of the IgG2 class. The humanized antibody may comprise sequences from more than one class or isotope, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework region (FR) and CDR sequences, more often 90%, and most preferably greater than 95%. Humanized antibody can be produced using variety of techniques known in the art, including but not limited to, CDR grafting (European Patent No. EP 239,400; International Publication No. WO 91/09967; and U.S. Pat. Nos. 5,225,539; 5,530,101; and 5,585,089), veneering or resurfacing (European Patent Nos. EP 592,106 and EP 519,596; Padlan, 1991, Molecular Immunology 28(415):489 498; Studnicka et al., 1994, Protein Engineering 7(6):805 814; and Roguska et al., 1994, PNAS 91:969 973), chain shuffling (U.S. Pat. No. 5,565,332), and techniques disclosed in, e.g., U.S. Pat. Nos. 6,407,213; 5,766,886; WO 9317105; Tan et al., J. Immunol. 169:1119-25 (2002); Caldas et al., Protein Eng. 13(5):353-60 (2000); Morea et al., Methods 20(3):267-79 (2000); Baca et al., J. Biol. Chem. 272(16):10678-84 (1997); Roguska et al., Protein Eng. 9(10):895-904 (1996); Couto et al., Cancer Res. 55 (23 Supp):5973s-5977s (1995); Couto et al., Cancer Res. 55(8):1717-22 (1995); Sandhu J S, Gene 150(2):409-10 (1994); and Pedersen et al., J. Mol. Biol. 235(3):959-73 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323).

Also provided is a cell comprising one or more of the herein provided vectors. As used herein, “cell”, “cell line”, and “cell culture” may be used interchangeably and all such designations include progeny. The disclosed cell can be any cell used to clone or propagate the vectors provided herein. Thus, the cell can be from any primary cell culture or established cell line. The method may be applied to any cell, including prokaryotic or eukaryotic, such as bacterial, plant, animal, and the like. The cell type can be selected by one skilled in the art based on the choice of vector and desired use.

Disclosed are animals produced by the process of transfecting a cell within the animal with any of the nucleic acid molecules or vectors disclosed herein. Disclosed are animals produced by the process of transfecting a cell within the animal any of the nucleic acid molecules or vectors disclosed herein, wherein the animal is a mammal. Also disclosed are animals produced by the process of transfecting a cell within the animal any of the nucleic acid molecules or vectors disclosed herein, wherein the mammal is mouse, rat, rabbit, cow, sheep, pig, or primate.

Provided is a composition comprising one or more of the herein provided polypeptides, nucleic acids, or vectors in a pharmaceutically acceptable carrier. Thus, provided is a composition comprising a combination of two or more of any of the herein provided ACT polypeptides in a pharmaceutically acceptable carrier. For example, provided is a composition comprising SEQ ID NO:1 and SEQ ID NO:5 in a pharmaceutically acceptable carrier.

By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

In another embodiment, the polypeptides, nucleic acids, vectors, and/or host cells of this disclosure or administered with at least one cancer therapeutic agent. The polypeptides, nucleic acids, vectors, and/or host cells and the cancer chemotherapeutic agent may be administered separately or formulated together. One embodiment of this disclosure comprises a composition comprising a polypeptide comprising the carboxy-terminal amino acid sequence of an alpha connexin, or a conservative variant thereof and a cancer chemotherapeutic agent. Another embodiment of this disclosure comprises a composition comprising a nucleic acid encoding the carboxy-terminal amino acid sequence of an alpha connexin, or a conservative variant thereof and a cancer chemotherapeutic agent. Another embodiment of this disclosure comprises a composition comprising a nucleic acid encoding the carboxy-terminal amino acid sequence of an alpha connexin, or a conservative variant thereof and a cancer chemotherapeutic agent. Another embodiment of this disclosure comprises a composition comprising a recombinant vector expressing the carboxy-terminal amino acid sequence of an alpha connexin, or a conservative variant thereof and a cancer chemotherapeutic agent. In one particular embodiment, the cancer chemotherapeutic agent is Temozolomide.

The cancer chemotherapeutic agent may be selected from the group consisting of Abiraterone Acetate, ABITREXATE (Methotrexate), ABRAXANE (Paclitaxel Albumin-stabilized Nanoparticle Formulation), ADCETRIS (Brentuximab Vedotin), Ado-Trastuzumab Emtansine, ADRIAMYCIN (Doxorubicin Hydrochloride), ADRUCIL (Fluorouracil), Afatinib Dimaleate, AFINITOR (Everolimus), ALDARA (Imiquimod), Aldesleukin, Alemtuzumab, ALIMTA (Pemetrexed Disodium), ALOXI (Palonosetron Hydrochloride), AMBOCHLORIN (Chlorambucil), AMBOCLORIN (Chlorambucil), Aminolevulinic Acid, Anastrozole, Aprepitant, AREDIA (Pamidronate Disodium), ARIMIDEX (Anastrozole), AROMASIN (Exemestane), ARRANON (Nelarabine), Arsenic Trioxide, ARZERRA (Ofatumumab), Asparaginase Erwinia chrysanthemi, AVASTIN (Bevacizumab), Axitinib, Azacitidine, Bendamustine Hydrochloride, Bevacizumab, Bexarotene, BEXXAR (Tositumomab and I 131 Iodine Tositumomab), Bleomycin, Bortezomib, BOSULIF (Bosutinib), Cabazitaxel, Cabozantinib-S-Malate, CAM PATH (Alemtuzumab), CAMPTOSAR (Irinotecan Hydrochloride), Capecitabine, Carboplatin, Carfilzomib, CEENU (Lomustine), CERUBIDINE (Daunorubicin Hydrochloride), Cetuximab, Chlorambucil, Cisplatin, CLAFEN (Cyclophosphamide), Clofarabine, COMETRIQ (Cabozantinib-S-Malate), COSMEGEN (Dactinomycin), Crizotinib, Cyclophosphamide, CYFOS (Ifosfamide), Cytarabine, Dabrafenib, Dacarbazine, DACOGEN (Decitabine), Dactinomycin, Dasatinib, Daunorubicin Hydrochloride, Decitabine, Degarelix, Denileukin Diftitox, Denosumab, Dexrazoxane Hydrochloride, Docetaxel, Doxorubicin Hydrochloride, EFUDEX (Fluorouracil), ELITEK (Rasburicase), ELLENCE (Epirubicin Hydrochloride), ELOXATIN (Oxaliplatin), Eltrombopag Olamine, EMEND (Aprepitant), Enzalutamide, Epirubicin Hydrochloride, ERBITUX (Cetuximab), Eribulin Mesylate, ERIVEDGE (Vismodegib), Erlotinib Hydrochloride, ERWINAZE (Asparaginase Erwinia chrysanthemi), Etoposide, Everolimus, EVISTA (Raloxifene Hydrochloride), Exemestane, FARESTON (Toremifene), FASLODEX (Fulvestrant), FEMARA (Letrozole), Filgrastim, FLUDARA (Fludarabine Phosphate), Fludarabine Phosphate, FLUOROPLEX (Fluorouracil), Fluorouracil, Folinic acid, FOLOTYN (Pralatrexate), Fulvestrant, Gefitinib, Gemcitabine Hydrochloride, Gemtuzumab Ozogamicin, GEMZAR (Gemcitabine Hydrochloride), GILOTRIF (Afatinib Dimaleate), GLEEVEC (Imatinib Mesylate), HALAVEN (Eribulin Mesylate), HERCEPTIN (Trastuzumab), HYCAMTIN (Topotecan Hydrochloride), Ibritumomab Tiuxetan, ICLUSIG (Ponatinib Hydrochloride), Ifosfamide, Imatinib Mesylate, Imiquimod, INLYTA (Axitinib), INTRON A (Recombinant Interferon Alfa-2b), Iodine 131 Tositumomab and Tositumomab, Ipilimumab, IRESSA (Gefitinib), Irinotecan Hydrochloride, ISTODAX (Romidepsin), Ixabepilone, JAKAFI (Ruxolitinib Phosphate), JEVTANA (Cabazitaxel), Kadcyla (Ado-Trastuzumab Emtansine), KEOXIFENE (Raloxifene Hydrochloride), KEPIVANCE (Palifermin), KYPROLIS (Carfilzomib), Lapatinib Ditosylate, Lenalidomide, Letrozole, Leucovorin Calcium, Leuprolide Acetate, Lomustine, LUPRON (Leuprolide Acetate, MARQIBO (Vincristine Sulfate Liposome), MATULANE (Procarbazine Hydrochloride), Mechlorethamine Hydrochloride, MEGACE (Megestrol Acetate), Megestrol Acetate, MEKINIST (Trametinib), Mercaptopurine, Mesna, METHAZOLASTONE (Temozolomide), Methotrexate, Mitomycin, MOZOBIL (Plerixafor), MUSTARGEN (Mechlorethamine Hydrochloride), MUTAMYCIN (Mitomycin C), MYLOSAR (Azacitidine), MYLOTARG (Gemtuzumab Ozogamicin), Nanoparticle Paclitaxel (Paclitaxel Albumin-stabilized Nanoparticle Formulation), NAVELBINE (Vinorelbine Tartrate), Nelarabine, NEOSAR (Cyclophosphamide), NEUPOGEN (Filgrastim), NEXAVAR (Sorafenib Tosylate), Nilotinib, NOLVADEX (Tamoxifen Citrate), NPLATE (Romiplostim), Ofatumumab, Omacetaxine Mepesuccinate, ONCASPAR (Pegaspargase), ONTAK (Denileukin Diftitox), Oxaliplatin, Paclitaxel, Paclitaxel Albumin-stabilized Nanoparticle Formulation, Palifermin, Palonosetron Hydrochloride, Pamidronate Disodium, Panitumumab, Pazopanib Hydrochloride, Pegaspargase, Peginterferon Alfa-2b, PEG-INTRON (Peginterferon Alfa-2b), Pemetrexed Disodium, Pertuzumab, PLATINOL (Cisplatin), PLATINOL-AQ (Cisplatin), Plerixafor, Pomalidomide, POMALYST (Pomalidomide), Ponatinib Hydrochloride, Pralatrexate, Prednisone, Procarbazine Hydrochloride, PROLEUKIN (Aldesleukin), PROLIA (Denosumab), PROMACTA (Eltrombopag Olamine), PROVENGE (Sipuleucel-T), PURINETHOL (Mercaptopurine), Radium 223 Dichloride, Raloxifene Hydrochloride, Rasburicas, Recombinant Interferon Alfa-2b, Regorafenib, REVLIMID (Lenalidomide), RHEUMATREX (Methotrexate), Rituximab, Romidepsin, Romiplostim, RUBIDOMYCIN (Daunorubicin Hydrochloride), Ruxolitinib Phosphat, Sipuleucel-T, Sorafenib Tosylate, SPRYCEL (Dasatinib), STIVARGA (Regorafenib), Sunitinib Malate, SUTENT (Sunitinib Malate), SYLATRON (Peginterferon Alfa-2b), SYNOVIR (Thalidomide), SYNRIBO (Omacetaxine Mepesuccinate), TAFINLAR (Dabrafenib), Tamoxifen Citrate, TARABINE PFS (Cytarabine), TARCEVA (Erlotinib Hydrochloride), TARGRETIN (Bexarotene), TASIGNA (Nilotinib), TAXOL (Paclitaxel), TAXOTERE (Docetaxel), TEMODAR (Temozolomide), Temozolomide, Temsirolimus, Thalidomide, TOPOSAR (Etoposide), Topotecan Hydrochloride, Toremifene, TORISEL (Temsirolimus), Tositumomab and I 131 Iodine Tositumomab, TOTECT (Dexrazoxane Hydrochloride), Trametinib, Trastuzumab, TREANDA (Bendamustine Hydrochloride), TRISENOX (Arsenic Trioxide), TYKERB (Lapatinib Ditosylate), Vandetanib, VECTIBIX (Panitumumab), VeIP, VELBAN (Vinblastine Sulfate), VELCADE (Bortezomib), VELSAR (Vinblastine Sulfate), Vemurafenib, VEPESID (Etoposide), VIADUR (Leuprolide Acetate), VIDAZA (Azacitidine), Vinblastine Sulfate, Vincristine Sulfate, Vinorelbine Tartrate, Vismodegib, VORAXAZE (Glucarpidase), Vorinostat, VOTRIENT (Pazopanib Hydrochloride), WELLCOVORIN (Leucovorin Calcium), XALKORI (Crizotinib), XELODA (Capecitabine), XGEVA (Denosumab), XOFIGO (Radium 223 Dichloride), XTANDI (Enzalutamide), YERVOY (Ipilimumab), ZALTRAP (Ziv-Aflibercept), ZELBORAF (Vemurafenib), ZEVALIN (Ibritumomab Tiuxetan), ZINECARD (Dexrazoxane Hydrochloride), Ziv-Aflibercept, Zoledronic Acid, ZOLINZA (Vorinostat), ZOMETA (Zoledronic Acid), and ZYTIGA (Abiraterone Acetate), including any formulation (e.g. liposomal, pegylated) any salt or any brand name of any generic agent included herein.

In another embodiment, the polypeptides, nucleic acids, vectors, and/or host cells of this disclosure are administered with radiation therapy, alternatively or in addition to cancer chemotherapy. Radiation therapy treatment for glioma at a total dose of 50-65 Gy in fraction sizes of 1.8-2.0 Gy has been recommended (see Laperriere N et al., Radiother Oncol. 2002 September; 64(3):259-73).

The compositions may be administered topically, orally, or parenterally. For example, the compositions can be administered extracorporeally, intracranially, intravaginally, intraanally, subcutaneously, intradermally, intracardiac, intragastric, intravenously, intramuscularly, by intraperitoneal injection, transdermally, intranasally, or by inhalation. As used herein, “intracranial administration” means the direct delivery of substances to the brain including, for example, intrathecal, intracisternal, intraventricular or trans-sphenoidal delivery via catheter or needle.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

The exact amount of the compositions required will vary from subject to subject, depending on the species, age, weight and general condition of the subject, the severity of the glioma being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

The materials may be in solution or suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution can be from about 5 to about 8, from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels (e.g., poloxamer gel), drops, controlled-release compositions, timed release compositions, suppositories, sprays, liquids and powders. Conventional pharmaceutical carriers, aqueous, powder or oily bases, thickeners and the like may be necessary or desirable. The disclosed compositions can be administered, for example, in a microfiber, polymer (e.g., collagen), glasses, nanosphere, aerosol, lotion, cream, fabric, plastic, tissue engineered scaffold, matrix material, tablet, implanted container, powder, oil, resin, wound dressing, bead, microbead, slow-release compounds, timed-release compounds, capsule, injectables, intravenous drips, pump device, silicone implants, or any bio-engineered materials.

Compositions for oral administration include powders or granules, suspensions or solutions in water or non-aqueous media, capsules, sachets, or tablets. Thickeners, flavorings, diluents, emulsifiers, dispersing aids or binders may be desirable. Pharmaceutically acceptable carriers include fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are flow-regulating agents and lubricants, for example, silica, talc, stearic acid or salts thereof, such as magnesium stearate or calcium stearate, and/or polyethylene glycol. In one embodiment, dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethylcellulose phthalate, are used. Slow dissolving polymers such as poly(bis(p-carboxyphenoxy)-propane:sebacic acid—CCP:SA) may also be used to generate wafers or beads that control or time the release of the composition. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules or nanoparticles which may optionally be mixed with fillers such as lactose, binders such as starches, and/or lubricants such as talc or magnesium stearate and, optionally, stabilizers. In one embodiment, the peptides of this disclosure are dissolved or suspended in suitable liquids, such as fatty oils, or liquid paraffin, optionally with stabilizers.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Fatty oils may comprise mono-, di- or triglycerides. Mono-, di- and triglycerides include those that are derived from C6, C8, C10, C12, C14, C16, C18, C20 and C22 acids. Exemplary diglycerides include, in particular, diolein, dipalmitolein, and mixed caprylin-caprin diglycerides. Preferred triglycerides include vegetable oils, fish oils, animal fats, hydrogenated vegetable oils, partially hydrogenated vegetable oils, synthetic triglycerides, modified triglycerides, fractionated triglycerides, medium and long-chain triglycerides, structured triglycerides, and mixtures thereof. Exemplary triglycerides include: almond oil; babassu oil; borage oil; blackcurrant seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; partially soy and cottonseed oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; and glyceryl tricaprylate/caprate/stearate.

In one embodiment, the triglyceride is the medium chain triglyceride available under the trade name LABRAFAC CC. Other triglycerides include neutral oils, e.g., neutral plant oils, in particular fractionated coconut oils such as known and commercially available under the trade name MIGLYOL, including the products: MIGLYOL 810; MIGLYOL 812; MIGLYOL 818; and CAPTEX 355. Other triglycerides are caprylic-capric acid triglycerides such as known and commercially available under the trade name MYRITOL, including the product MYRITOL 813. Further triglycerides of this class are CAPMUL MCT, CAPTEX 200, CAPTEX 300, CAPTEX 800, NEOBEE M5 and MAZOL 1400.

Pharmaceutical compositions comprising triglycerides may further comprise lipophilic and/or hydrophilic surfactants which may form clear solutions upon dissolution with an aqueous solvent. One such surfactant is tocopheryl polyethylene glycol 1000 succinate (vitamin E TPGS). Examples of such compositions are described in U.S. Pat. No. 6,267,985.

In another embodiment, the peptides of this disclosure (such as ACT1) may be delivered in alginate-poly-l-lysine and alginate-poly-l-ornithine coated microcapsules to circumvent rapid diffusion and loss of peptide in initial time points, controlling the release of the peptide as described in (Moore K, Ghatnekar G, Gourdie R G, Potts J D. Impact of the controlled release of a connexin 43 peptide on corneal wound closure in an STZ model of type I diabetes. PLoS One. 2014 Jan. 23; 9(1):e86570; Moore K, Amos J, Davis J, Gourdie R, Potts J D. Characterization of polymeric microcapsules containing a low molecular weight peptide for controlled release. Microsc Microanal. 2013 February; 19(1):213-26).

In yet another embodiment poly 1,3-bis-(p-carboxyphenoxy) propane-co-sebacic acid (p(CPP:SA)) microspheres or wafers can be fabricated containing the peptide compounds using methods including the water-in-oil-in-water (w/o/w) double emulsion solvent evaporation technique as described in MANOHARAN C and SINGH J, Evaluation of Polyanhydride Microspheres for Basal Insulin Delivery: Effect of Copolymer Composition and Zinc Salt on Encapsulation, In Vitro Release, Stability, In Vivo Absorption and Bioactivity in Diabetic Rats, JOURNAL OF PHARMACEUTICAL SCIENCES, VOL. 98, NO. 11, NOVEMBER 2009. Such compositions can be manufactured in such a manner to control or time release of the compound over time periods of up to a month. The controlled release vehicles can be delivered by intracranial injection directly in or around tumors or placed into the organ in the tissue bed generated by surgical resection of the tumor.

Another embodiment may include versions of the peptides that have been optimized to prolong their half-life. Optimized versions may include blocking the peptide by the addition of an n-terminal amide group and a c-terminal acetyl group; pegylation or linkage to other polymers; fusion to albumin, the Fc fragment of immunoglobulin, or other proteins with a long half-life; cyclization; and the like. For example, Fc fragment of human IgG or inert polymer molecules such as high molecular weight polyethyleneglycol (PEG) can be attached to a peptide of this disclosure or an analog or derivative thereof with or without a multifunctional linker either through site-specific conjugation of the PEG to the N- or C-terminus of the protein or via epsilon-amino groups present on lysine residues. Linear or branched polymer derivatization that results in minimal loss of biological activity can be used. The degree of conjugation can be closely monitored by SDS-PAGE and mass spectrometry to ensure proper conjugation of PEG molecules. Unreacted PEG can be separated from peptide-PEG conjugates by size-exclusion or by ion-exchange chromatography. PEG-derivatized conjugates can be tested for in vivo efficacy using methods known to those of skill in the art. The half-life of peptides of this disclosure may be extended by any method known in the art. Optimized versions may also include peptidomimetics based on the peptides.

Possible pharmaceutical preparations which can be used rectally include, for example, suppositories, which consist of a combination of one or more of the peptides of this disclosure with a suppository base. Suitable suppository bases are, for example, natural or synthetic triglycerides, or paraffin hydrocarbons. In addition, it is also possible to use gelatin rectal capsules which consist of the peptides of this disclosure with a base. Possible base materials include, for example, liquid triglycerides, polyethylene glycols, or paraffin hydrocarbons.

An exemplary composition comprises 100 mg of sterile and pyrogen-free temozolomide lyophilized powder and 100 mg ACT-1 peptide (SEQ ID NO:2) in a vial for intravenous, intrathecal, intracisternal, intraventricular or trans-sphenoidal injection. The vial contains inactive ingredients including mannitol (600 mg), L-threonine (160 mg), polysorbate 80 (120 mg), sodium citrate dihydrate (235 mg), and hydrochloric acid (160 mg). When reconstituted with 41 mL Sterile Water for Injection, the resulting solution will contain 2.5 mg/mL temozolomide and 2.5 mg/ml ACT-1.

Another exemplary composition comprises 100 mg temozolomide and 10 mg AAV vector with a polynucleotide sequence encoding ACT-1 peptide (SEQ ID NO:2) in 41 ml Sterile Water in a vial for intravenous, intrathecal, intracisternal, intraventricular or trans-sphenoidal injection. The vial contains inactive ingredients including mannitol (600 mg), L-threonine (160 mg), polysorbate 80 (120 mg), sodium citrate dihydrate (235 mg), and hydrochloric acid (160 mg). The solution contains 2.5 mg/ml temozolomide and 0.25 mg/ml ACT-1.

Another exemplary composition comprises 100 mg of sterile and pyrogen-free cisplatin lyophilized powder and 200 mg ACT-1 peptide (SEQ ID NO:2) in a vial for intravenous, intrathecal, intracisternal, intraventricular or trans-sphenoidal injection. The vial contains inactive ingredients including mannitol (600 mg), L-threonine (160 mg), polysorbate 80 (120 mg), sodium citrate dihydrate (235 mg), and hydrochloric acid (160 mg). When reconstituted with 41 mL Sterile Water for Injection, the resulting solution will contain 2.5 mg/mL temozolomide and 5.0 mg/ml ACT-1. Similar exemplary compositions with other cancer chemotherapeutic agents may be envisioned.

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual doctor in the event of any counter indications. Dosage can vary, and can be administered in one or more dose administrations daily, for one or several days. Guidance can be found in the literature for appropriate dosages for given classes of pharmaceutical products. The range of dosage largely depends on the application of the compositions herein, severity of condition, and its route of administration.

For example, in applications as a laboratory tool for research, the ACT peptide compositions can be used in doses as low as 0.01% w/v. Significantly higher concentrations of the compositions by themselves or in combination with other compounds may be used in applications like cancer/tumor therapy. Thus, upper limits of the provided polypeptides may be up to 2-5% w/v or v/v if given as an initial bolus delivered for example directly into a tumor mass. Recommended upper limits of dosage for parenteral routes of administration for example intramuscular, intracerebral, intracardicardiac and intraspinal could be up to 1% w/v or v/v depending on the severity of the injury. This upper dosage limit may vary by formulation, depending for example on how the polypeptide(s) is combined with other agents promoting its action or acting in concert with the polypeptide(s).

For continuous delivery of the provided polypeptides, for example, in combination with an intravenous drip, upper limits of 0.01 g/Kg body weight over time courses determined by the doctor based on improvement in the condition can be used. In another example, upper limits of concentration of the provided nucleic acids delivered internally for example, intramuscular, intracerebral, intracardicardiac and intraspinal would be 50-100 μg/ml of solution. Again, the frequency would be determined by the Doctor based on improvement.

Viral vectors remain highly experimental tools that nonetheless show considerable potential in clinical applications. As such, caution is warranted in calculation of expected dosage regimes for viral vectors and will depend considerably on the type of vector used. For example, retroviral vectors infect dividing cells such as cancer cells efficiently, intercalating into the host cell genome and continuing expression of encoded proteins indefinitely. Typical dosages of retroviruses in an animal model setting are in the range of 107 to 109 infectious units per ml. By contrast, adenoviruses most efficiently target post-mitotic cells, but cells are quickly eliminated by the host immune system or virus is eventually lost if infected cells resume proliferation and subsequently dilute the viral episomal DNA. Indeed, this transient time course of infection may be useful for short-term delivery of the composition described herein in certain clinical situations, for example in amelioration of a small injury. In animal models, concentrations of 10⁸-10¹¹ infectious units per ml of adenovirus are typical for uses in research. Dose ranges of vectors based on data derived from animal models would be envisaged to be used eventually in clinical setting(s), pending the development of pharmaceutically acceptable formulation(s).

An exemplary embodiment of this disclosure includes a recombinant vector expressing one or more of the peptides of this disclosure. The recombinant vector may be cDNA, lentivirus, adenovirus, adeno-associated virus or retrovirus. The recombinant vector may express a polypeptide comprising a carboxy-terminal amino acid sequence of alpha-connexin and a secretory signal peptide, IL13 receptor peptide, Fc fragment, or MMP cleavage domain. The recombinant vector may be administered intracranially, intravenously, or intratumorally, and may be coadministered with a chemotherapeutic agent or radiation or before, during, or after surgery or as an alternative to surgery.

In one embodiment, the recombinant vector comprises an inducible gene switch. The gene switch may be any of a variety of inducible promoter systems that are available to a skilled artisan. Exemplary gene switches that may be used include those activated by tetracycline (TET-ON), rapamycin or its derivatives, or steroid hormones such as ecdysone or its derivatives. However, it is preferred that the ligand of the inducible promoter systems have minimal to negligible toxicity in humans. The gene switch may be any gene switch that regulates gene expression by addition or removal of a specific ligand. In one embodiment, the gene switch is one in which the level of gene expression is dependent on the level of ligand that is present. Examples of ligand-dependent transcription factor complexes that may be used in the gene switches of the invention include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In one aspect of the invention, the gene switch is an EcR-based gene switch. Examples of such systems include, without limitation, the systems described in U.S. Pat. Nos. 6,258,603, and 7,045,315, U.S. Published Patent Application Nos. 2006/0014711, 2007/0161086, and International Published Application No. WO 01170816. Examples of chimeric ecdysone receptor systems are described in U.S. Pat. No. 7,091,038, U.S. Published Patent Application Nos. 2002/0110861, 2004/0033600, 2004/0096942, 2005/0266457, and 2006/0100416, and International Published Application Nos. WO 01170816, WO25 02/066612, WO 02/066613, WO 02/066614, WO 02/066615, WO 02129075, and WO 20051108617, each of which is incorporated by reference in its entirety. In another aspect of the invention, the gene switch is based on heterodimerization of FK506 binding 30 protein (FKBP) with FKBP rapamycin associated protein (FRAP) and is regulated through rapamycin or its non-immunosuppressive analogs. Examples of such systems, include, without limitation, the ARGENT™ Transcriptional Technology (ARIAD Pharmaceuticals, Cambridge, Mass.) and the systems described in U.S. Pat. Nos. 6,015,709, 6,117,680, 6,479,653, 6,187,757, and 6,649,595. Other embodiments of the gene switch may include a procaryotic repressor/operator-based gene switch system, such as a Tet or Lac-based system. In one example of a Tet system, tetracycline binds to the TetR repressor protein, resulting in a conformational change which releases the repressor protein from the operator which as a result allows transcription to occur. In another example of a Tet system, Tet-On 3G (a modified form of the Tet-On Advanced transactivator protein which has been evolved to display far higher sensitivity to doxycycline) binds specifically to the inducible promoter PTRE3GS and activates transcription of the downstream gene of interest in the presence of doxycycline. A commercially-available Tet gene expression system based on this mode of operation is the Lenti-X™ Tet-On® 3G developed by Clontech Laboratories, Inc. (Mountain View, Calif.). In the Lac system, a lac operon is inactivated in the absence of lactose, or synthetic analogs such as isopropyl-b-D-thiogalactoside.

Additional gene switch systems that may be used include those described in: U.S. Pat. No. 7,091,038; WO2004078924; EP1266015; US20010044151; US20020110861; US20020119521; US20040033600; US20040197861; US20040235097; US20060020146; US20040049437; US20040096942; US20050228016; US20050266457; US20060100416; WO2001170816; WO2002129075; WO2002/066612; WO2002/066613; WO2002/066614; WO2002/066615; WO20051108617; U.S. Pat. No. 6,258,603; US20050209283; US20050228016; US20060020146; EP0965644; U.S. Pat. No. 7,304,162; U.S. Pat. No. 7,304,161; MX234742; KRI0-0563143; AU765306; AU2002-248500; and AU2002-306550.

The vectors may be used to transform host cells that may be used as a therapeutic agent for expressing the peptides. In one embodiment, the host cells are mesenchymal stem cells. Mesenchymal stem cells (MSCs) are multipotent mesoderm-derived progenitor cells. The main source known of MSCs in adult humans is the bone marrow compartment that contains several cell types including cells of the hematopoietic lineage, endothelial cells, and mesenchymal stem cells, which are part of the marrow stromal system (Pittenger et al, 1999). Positive and negative selection markers for mesenchymal stem cells are known (see Calloni R, Reviewing and updating the major molecular markers for stem cells, Stem Cells Dev. 2013 May 1; 22(9):1455-76). Positive selection markers for mesenchymal stem cells include CD13, CD29, CD44, CD49e, CD54, CD71, CD73, CD90, CD105, CD106, CD166, and HLA-ABC. Negative selection markers for mesenchymal stem cells include CD14, CD31, CD34, CD45, CD62E, CD62L, CD62P, and HLA-DR. Human mesenchymal stem cells can be isolated through flow cytometry techniques that exploit these markers, and a number of kits for this purpose that contain antibodies to these markers are commercially available, such as the Human Mesenchymal Stem Cell Lineage Antibody Cocktail, Cat No. 562530, available from BD Biosciences (San Jose, Calif.).

In one embodiment, the peptide may be manufactured as a chimeric peptide that includes a protein that targets glioma cells. IL13 is an example of one such protein as IL13 receptor is overexpressed in glioma cells. A chimeric protein with IL13 and a carboxy terminal fragment of alpha connexin is one embodiment of a chimeric protein that is engineered to target glioma cells.

In other embodiments, the host cells are liver stem cells, mammary stem cells, pancreatic stem cells, neuronal stem cells, and embryonic stem cells. Other markers are known for these stem cell lines, and can be similarly exploited with flow cytometry techniques to isolate these cells. The stem cells may or may not be pluripotent. “Pluripotent cells” include cells and their progeny, which may be able to differentiate into, or give rise to, pluripotent, multipotent, oligopotent and unipotent cells. “Multipotent cells” include cells and their progeny, which may be able to differentiate into, or give rise to, multipotent, oligopotent and unipotent progenitor cells, and/or one or more mature or partially mature cell types, except that the mature or partially mature cell types derived from multipotent cells are limited to cells of a particular tissue, organ or organ system. As used herein, “partially mature cells” are cells that exhibit at least one characteristic of the phenotype, such as morphology or protein expression, of a mature cell from the same organ or tissue. For example, a multipotent hematopoietic progenitor cell and/or its progeny possess the ability to differentiate into or give rise to one or more types of oligopotent cells, such as myeloid progenitor cells and lymphoid progenitor cells, and also give rise to other mature cellular components normally found in the blood. “Oligopotent cells” include cells and their progeny whose ability to differentiate into mature or partially mature cells is more restricted than multipotent cells. Oligopotent cells may, however, still possess the ability to differentiate into oligopotent and unipotent cells, and/or one or more mature or partially mature cell types of a given tissue, organ or organ system. One example of an oligopotent cell is a myeloid progenitor cell, which can ultimately give rise to mature or partially mature erythrocytes, platelets, basophils, eosinophils, neutrophils and monocytes. “Unipotent cells” include cells and their progeny that possess the ability to differentiate or give rise to other unipotent cells and/or one type of mature or partially mature cell type. As used herein, the term “progenitor cell” can mean cells and their progeny that differentiate into at least partially mature cells, but lack the capacity for indefinite self-renewal in culture. Progenitor cells, as used herein, may be pluripotent, multipotent, oligopotent or even unipotent.

In an exemplary embodiment, mesenchymal stem cells (MSCs) are transformed with a vector which expresses a chimeric peptide of this disclosure under the control of an inducible gene expression system. The MSCs can be administered systemically and may cross the blood-brain-barrier from blood circulation and home to glioma cells. A ligand that activates the inducible gene expression system (e.g. doxycycline in a Tet-On system) may be administered to the patient to control timing of expression of the peptide. In this way, expression of a carboxy-terminal amino acid sequence of alpha connexin in the brain may be controlled through administration of a ligand such as doxycycline.

A. Methods

Embodiments of this disclosure provide a method of treating glioma in a subject, comprising administering to the subject one or more of the herein provided compositions (e.g., polypeptides, nucleic acids, vectors, and/or host cells) in a pharmaceutically acceptable carrier. Further, embodiments of this disclosure provide a method of treating glioma in a subject, comprising administering to the subject one or more of the herein provided compositions (e.g. polypeptides, nucleic acid, vectors, and/or host cells) which includes a chemotherapeutic agent or is administered in combination with a chemotherapeutic agent. Further, embodiments of this disclosure provide a method of treating or preventing glioblastoma multiforme in a subject, comprising administering to the subject a recombinant vector expressing a polypeptide comprising the carboxy-terminal amino acid sequence of an alpha connexin, or a conservative variant thereof and temozolomide. Further embodiments of this disclosure provide a composition comprising a compound that interacts with the carboxy-terminal amino acid sequence of alpha connexin and temozolomide or a method of treating or preventing glioblastoma multiforme in a subject, comprising administering to the subject a compound that interacts with the carboxy-terminal amino acid sequence of alpha connexin and temozolomide. The compound that interacts with the carboxy-terminal amino acid sequence of alpha connexin may be ACT peptide or AAP10 (Accession No. NP_001185877). In other embodiments, the compound may be H2 peptide, GAP19, GAP134, ZP123, danepeptide, rotigaptide, or RXP-E.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, the increase can be a 10, 20, 30, 40, 50, 60, 70, 80, 90, 100%, or any amount of increase in between as compared to native or control levels.

By “treat” or “treatment” is meant a method of reducing the effects of a disease or condition, or administering a composition for the purpose of reducing such effects. Treatment can also refer to a method of reducing the underlying cause of the disease or condition itself rather than just the symptoms. The treatment can be any reduction from native levels and can be but is not limited to the complete ablation of the disease, condition, or the symptoms of the disease or condition. For example, a disclosed method for treating glioma is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. Further, the efficacy of treatment may be measured as a 10, 20, 30, 40, 50, 60, 70, 80, 90, 100%, or any amount of reduction in between of the radius, diameter, or volume of a glioma tumor in comparison to that before treatment within a subject or a similar reduction in comparison to the radius, diameter, or volume of a glioma tumor in one or more control subjects. Further, the efficacy of treatment may be measured in terms of a progression-free survival period, or overall survival. For example, The North American Brain Tumor Consortium (NABTC) uses 6-month progression-free survival (6moPFS) as the efficacy end point of therapy trials for adult patients with recurrent high-grade gliomas (see Lamborn K R, Progression-free survival: An important end point in evaluating therapy for recurrent high-grade gliomas, Neuro Oncol (April 2008) 10 (2):162-170).

Another embodiment of this disclosure provides a method of treating or preventing glioblastoma multiforme in a subject, comprising administering to the subject a recombinant vector expressing a polypeptide that interacts with the carboxy-terminal amino acid sequence of alpha connexin Cx43 or a conservative variant thereof and temozolomide. Another embodiment of this disclosure provides a method of treating or preventing glioblastoma multiforme in a subject, comprising administering to the subject a compound that inhibits alpha connexin function and temozolomide. In another embodiment of this disclosure, the alpha connexin function inhibitor is selected from the group consisting of Cx43 antisense RNA, Cx43 siRNA inhibitor, Cx43 shRNA inhibitor, Cx43 microRNA inhibitor, JM peptide (see WO 2013163423 A1), GAP26, GAP27, heptanol, octanol anesthetics; halothane, propofol, ethflurane, flufenamic acid, 18-beta-glycyrrhetinic acid, and derivatives thereof; lysophosphatidic acid; lindane; mefloquine; okadaic acid; oleamide; quinidine; quinine; all trans-retinoic acid; vitamin A and retinoic acid derivatives and tamoxifen.

Another embodiment of this disclosure provides a method of treating or preventing glioblastoma multiforme in a subject, comprising administering to the subject a compound that promotes the bystander effect and temozolomide. The bystander effect involves the transfer of toxic compounds from one cell to a generally adjacent other through gap junction channels and through extracellular routes. Examples of compounds that act through the bystander effect include chemotherapeutic agents such as ganciclovir (see Jiang J X, Gap junction- and hemichannel-independent actions of connexins, Biochim Biophys Acta. 2005 Jun. 10; 1711(2):208-14) gemcitabine (see Laura Garcia-Rodriguez, Connexin-26 is a key factor mediating gemcitabine bystander effect, Molecular Cancer Therapeutics; 10(3):505-17) chloroethylnitrosoureas (see Merle P, Chemotherapy-induced bystander effect in response to several chloroethylnitrosoureas: an origin independent of DNA damage?, Anticancer Res. 2008 January-February; 28(1A):21-7), bleomycin, and neocarzinostatin (see Chinnadurai et al, Bleomycin, neocarzinostatin and ionising radiation-induced bystander effects in normal diploid human lung fibroblasts, bone marrow mesenchymal stem cells, lung adenocarcinoma cells and peripheral blood lymphocytes, International Journal of Radiation Biology, July 2011, Vol. 87, No. 7, Pages 673-682). Ionizing radiation is another well-known example of a therapy that acts through the bystander effect.

Another embodiment of this disclosure provides a method of treating or preventing cancer in a subject, comprising administering to the subject a polypeptide comprising the carboxy-terminal amino acid sequence of an alpha connexin, or a conservative variant thereof and temozolomide. Another embodiment of this disclosure provides a method of treating or preventing cancer in a subject, comprising administering to the subject a polypeptide comprising the carboxy-terminal amino acid sequence of an alpha connexin, or a conservative variant thereof and an anti-cancer therapy. Another embodiment of this disclosure provides a method of treating or preventing cancer in a subject, comprising administering to the subject a recombinant vector expressing a polypeptide comprising the carboxyl-terminal amino acid sequence of an alpha connexin, or a conservative variant thereof and an anti-cancer therapy.

In one embodiment, the cancer that may be treated according to this disclosure is a “high-grade” astrocytoma, such as malignant astrocytoma (grade III astrocytoma) and glioblastoma (grade IV astrocytoma). In another embodiment, the cancer that may be treated may include any brain tumor, including without limitation, Astrocytic tumors (e.g. Subependymal giant cell astrocytoma, Pilocytic astrocytoma, Pilomyxoid astrocytoma, Diffuse astrocytoma, Pleomorphic xanthoastrocytoma, Anaplastic astrocytoma, Glioblastoma, Giant cell glioblastoma, Gliosarcoma), Oligondendroglial tumors (e.g. Oligodendroglioma, Anaplastic oligodendroglioma), Oligoastrocytic tumors (e.g. Oligoastrocytoma, Anaplastic oligoastrocytoma), Ependymal tumor (e.g. Subependymoma, Myxopapillary ependymoma, Ependymoma, Anaplastic ependymoma), Choroid plexus tumors (e.g. Choroid plexus papilloma, Atypical choroid plexus papilloma, Choroid plexus carcinoma), Other neuroepithelial tumors (e.g. Angiocentric glioma, Chordoid glioma of the third ventricle), Neuronal and mixed neuronal-glial tumors (e.g. Gangliocytoma, Ganglioglioma, Anaplastic ganglioma, Desmoplastic infantile astrocytoma and ganglioglioma, Dysembryoplastic neuroepithelial tumor, Central neurocytoma, Extraventricular neurocytoma, Cerebellar liponeurocytoma, Paraganglioma of the spinal cord, Papillary glioneuronal tumor, Rosette-forming glioneural tumor of the fourth ventricle), Pineal tumors (e.g. Pineocytoma, Pineal parenchymal tumor of intermediate differentiation, Pineoblastoma, Papillary tumor of the pineal region), Embryonal tumors (e.g. Medulloblastoma, CNS primitive neuroectodermal tumor (PNET), Atypical teratoid/rhabdoid tumor) Tumors of the cranial and paraspinal nerves (e.g. Schwannoma, Neurofibroma, Perineurioma, Malignant peripheral nerve sheath tumor (MPNST), Meningeal tumors (e.g. Meningioma, Atypical meningioma, Anaplastic/malignant meningioma, Hemangiopericytoma, Anaplastic hemangiopericytoma, Hemangioblastoma), and tumors of the sellar region (e.g. Craniopharyngioma, Granular cell tumor of the neurohypophysis, Pituicytoma, Spindle cell oncocytoma of the adenohypophysis). In embodiments, the cancer that may be treated according to this disclosure is selected from the group consisting of breast cancer, colon cancer, rectal cancer, endometrial cancer, cervical cancer, kidney cancer, leukemia, liver cancer, stomach cancer, esophageal cancer, oral cancer, throat cancer, tracheal cancer, lung cancer, melanoma, non-melanoma skin cancers, non-Hodgkin lymphoma, Hodgkin lymphoma, pancreatic cancer, prostate cancer, head and neck cancers, bone cancer, and thyroid cancer.

Another embodiment of this disclosure comprises a method of treating or preventing disease in a subject, comprising administering to the subject a polypeptide comprising the carboxy-terminal amino acid sequence of an alpha connexin, or a conservative variant thereof and an anti-cancer therapy. Another embodiment of this disclosure comprises a method of treating or preventing disease in a subject, comprising administering a compound that interacts with the carboxy-terminal amino acid sequence of alpha connexin and an anti-cancer therapy. The anti-cancer therapy may be any anti-cancer therapy disclosed herein. In one embodiment, the disease is selected from a group consisting of ankylosing spondylitis, multiple sclerosis, Crohn's disease, diabetic autoimmune disease, autoimmune disease, wound healing, diabetic foot ulcers, venous leg ulcers, bed sores, scarring, fibrosis, keloid scarring, hypertrophic scarring, psoriasis, psoriatic arthritis, systemic lupus erythematosus, rheumatoid arthritis, and scleroderma.

One embodiment of the present disclosure provides a method of treating or preventing cancer in a subject, comprising administering to the subject a compound that interacts with the carboxy-terminal amino acid sequence of alpha connexin and an anti-cancer therapy. Another embodiment of the present disclosure provides a method of treating or preventing cancer in a subject, comprising administering to the subject a polypeptide that interacts with the carboxy-terminal amino acid sequence of alpha connexin and an anti-cancer therapy. Another embodiment of this disclosure comprises administering to the subject a recombinant vector expressing a polypeptide that interacts with the carboxy-terminal amino acid sequence of alpha connexin or a conservative variant thereof and an anti-cancer therapy.

In one embodiment of this disclosure, the polypeptide comprises an amino acid sequence with at least 65%, 70%, 75%, 80%, 85%, 90% sequence identity to RPRPDDLEI (SEQ ID NO:2).

In another embodiment of this disclosure, the polypeptide comprises an amino acid sequence with at least 65%, 70%, 75%, 80%, 85%, 90% sequence identity to RPRPDDELI (SEQ ID NO:92).

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. For treatment of brain tumors, administration may be though intracranial administration.

For systemic administration, the compositions can be formulated for transport across the blood-brain-barrier and/or glioma cell membranes. For example, the peptides, nucleic acids, or vectors of this disclosure can be formulated in liposomes. The liposomes may be further formulated with a targeting vector such as a protein, peptide, small molecules, or antibody. Alternatively, the peptides, nucleic acids, or vectors of this disclosure may be conjugated directly with a targeting vector. The targeting vector may exploit innate transport mechanisms in the blood brain barrier to transport the peptides, nucleic acids, and vectors of this disclosure. The peptides, nucleic acids, and vectors may be formulated to exploit receptor-mediated, magnetic directing, and cell-mediated drug delivery systems. For example, receptor mediated targeting may be exploited through the ligands for the transferrin receptor (see Tortorella S, The Significance of Transferrin Receptors in Oncology: the Development of Functional Nano-Based Drug Delivery Systems, Curr Drug Deliv. 2014 Jan. 5), the folate receptor (see Saul, J M, Controlled targeting of liposomal doxorubicin via the folate receptor, in vitro, Journal of Controlled Release 92 (2003) 49-67), IL-13 receptor, the epidermal growth factor receptor (EGF-R), the choline receptor (see Li J, Choline transporter-targeting and co-delivery system for glioma therapy, Biomaterials. 2013 December; 34(36):9142-8) to name a few. Cell surface receptors for malignant glioma have been characterized and are known in the art (see Li Y M, Cell surface receptors in malignant glioma, Neurosurgery. 2011 October; 69(4):980-94).

One example of a formulation for targeting the peptides, nucleic acids, or vectors of this disclosure to the blood-brain-barrier is a polymersome formulation. Polymersomes are described as bilayered vesicles capable of encapsulating both hydrophilic and hydrophobic drugs (see Krishnamoorthy B, Polymersomes as an effective drug delivery system for glioma—a review, J Drug Target. 2014 May 15:1-9). Another example of such a formulation is lactoferrin-modified poly(ethylene glycol)-grafted BSA nanoparticles (see Su Z, Lactoferrin-Modified Poly(ethylene glycol)-Grafted BSA Nanoparticles as a Dual-Targeting Carrier for Treating Brain Gliomas, Mol Pharm. 2014. Another example is a formulation comprising ligands of Interleukin 13 receptor α2 such as IL-13, Chitinase 3-like 1, or PEP-1 (see Wang B, Nanoparticles functionalized with Pep-1 as potential glioma targeting delivery system via interleukin 13 receptor α2-mediated endocytosis, Biomaterials. 2014 July; 35(22):5897-907). Another strategy is conjugation with bacteria cell surface proteins or their derivatives such as those described in US Patent Application Publication No. 20130004431A1. Another strategy is to administer vasoactive compounds intraarterially to increase blood-brain-barrier permeability (see Timothy E Cloughesy T E and Keith L. Black, Pharmacological blood-brain barrier modification for selective drug delivery, Journal of Neuro-Oncology 26: 125-132, 1995). Another strategy is to administer 5-phosphodiesterase inhibitors, such as sildenafil, vardenafil, or tadalafil, as described in International Patent Application Publication No. WO2006091542A2. Another strategy is to administer a nitric oxide synthase-3 inhibitor such as is described in U.S. Pat. No. 7,012,061. Another strategy is to conjugate the peptides, nucleic acids, and vectors of this disclosure with the peptides described in U.S. Pat. No. 7,399,747 B1. These strategies are merely listed as examples and are thus not intended to limit the skilled artisan from the variety of approaches available.

Alternatively, the peptides, nucleic acids, vectors, and/or host cells of this disclosure can be administered to bypass the blood-brain-barrier by intracranial administration, such as intraventricular, intrathecal, intracisternal, and trans-sphenoidal administration. Intraventricular administration is the administration of a substance into one of the ventricles of the brain, while intrathecal administration is administration introduced into or occurring in the space under the arachnoid membrane of the brain or spinal cord. Intracisternal administration is administration within one of the subarachnoid cisternae, while trans-sphenoidal administration is administration into the brain through the nose and the sphenoid bone. The peptides, nucleic acids, vectors, and/or host cells may be administered through any of these intracranial routes through any or any combination of a syringe, pump or implant.

B. Methods of Making the Compositions

The compositions disclosed herein and the compositions necessary to perform the disclosed methods can be made using any method known to those of skill in the art for that particular reagent or compound unless otherwise specifically noted.

For example, the provided nucleic acids can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N. Y., 1989) Chapters 5, 6) to purely synthetic methods, for example, by the cyanoethyl phosphoramidite method using a Milligen or Beckman System 1 Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

One method of producing the disclosed polypeptides, such as SEQ ID NO:2, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form a protein, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

Disclosed are processes for making the compositions as well as the intermediates leading to the compositions. There are a variety of methods that can be used for making these compositions, such as synthetic chemical methods and standard molecular biology methods. It is understood that the methods of making these and the other disclosed compositions are specifically disclosed. Disclosed are nucleic acid molecules produced by the process comprising linking in an operative way a nucleic acid encoding a polypeptide disclosed herein and a sequence controlling the expression of the nucleic acid. Disclosed are cells produced by the process of transforming the cell with any of the herein disclosed nucleic acids. Disclosed are any of the disclosed peptides produced by the process of expressing any of the herein disclosed nucleic acids. Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, dog, cat, rabbit, cow, sheep, pig, or primate. Also disclose are animals produced by the process of adding to the animal any of the cells disclosed herein.

### EXAMPLES

The following examples are intended to illustrate additional aspects of this disclosure. Examples described in the past tense should be construed as discussing the results of performed experiments, and examples described in the present or future tense should be construed as prophetic examples.

By way of background, Example 1 shows the results of a set of in vitro experiments, which show that ACT-1 and AAP10 interact with the CT of Cx43. Compounds that interact with Cx43 such as ACT-1 and AAP10 and disrupt its interaction with ZO-1 and its other binding partners can have the same efficacy on cancer and glioblastoma in combination with anti-cancer drugs such as temozolomide. Other examples of Cx43 CT binding compounds include H2 peptide (a peptide corresponding to amino acids 338 to 350 of Cx43 and conservative variants thereof), AAP10, GAP19, GAP134, ZP123, RRNYRRNY, danepeptide, rotigaptide, L2 peptide, RXP-E, and AAP10. Because, invasiveness is a complicated question, the inventors wanted to break it down into its principle components and probe them separately. In order to invade, cells must be able to adhere to the substrate into which they are invading. There has to be a finely-tuned balance between how much glioma cells adhere to each other in the tumor mass and how much they adhere to their surroundings. Another component of invasiveness is cell motility, which is dependent on the organization of the actin cytoskeleton, and finally, invasion, or protease-dependent breakdown of the surrounding matrix is also crucial to invasiveness. The first question the inventors asked was how the ACT-1 peptide affects glioma cell adhesion, and to answer that a cell aggregation assay was performed in Example 2. The second question the inventors asked was how the ACT-1 peptide affects glioma cell motility, and to answer that a cell motility assay was performed in Examples 3 and 4. Example 5 is a prophetic example to test the effects of ACT-1 on glioma cell invasion in a three-dimensional model of glioma. Example 6 describes the results of experiments testing the effects of treatments of ACT-1 or TMZ alone or their combination on the viability of a glioma stem cell line. Example 7 describes the results of experiments testing the effects of treatments of ACT-1 or TMZ alone or their combination in a sphere/colony formation assay. Example 8 describes the results of experiments testing the effects of treatments of ACT-1 or TMZ alone or their combination in a glioma xenograft mouse model. Example 9 shows the results of a preliminary study of IL13 peptide specifically delivered nanoparticles to human GBM xenografts. Example 10 is a prophetic example describing planned experiments to engineer rAAV vectors that express chimeric ACT-1 proteins, to determine the expression and activity of Chimeric ACT-1 proteins in vitro, and to measure the expression level and therapeutic effect of chimeric ACT-1 proteins in vivo. Example 11 is a prophetic example describing planned experiments to establish an MSC line expressing an inducible and active ACT-1 and to make MSC cell lines expressing i/+IL-13 or i/−IL-13.

In the following examples, ACT-1 has the amino acid sequence of SEQ ID NO:9 and the control peptide has reverse amino acid sequence of SEQ ID NO:9.

### Example 1

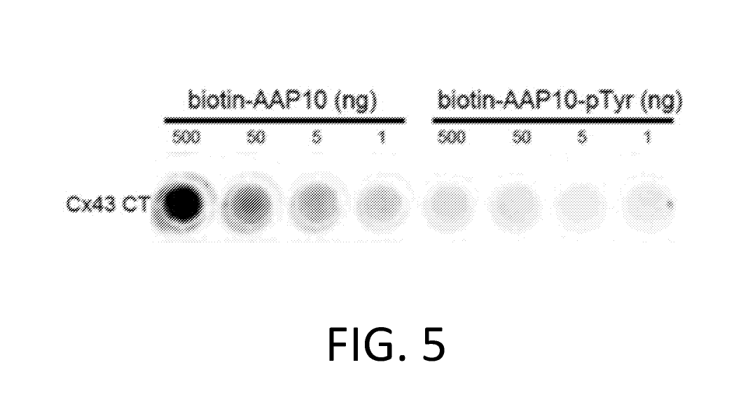

Unphosphorylated biotinylated AAP10 (500-1 ng) shows concentration dependent affinity for the Cx43 CT domain in a dot blot (FIG. 5). By contrast, the tyrosine phosphorylated isoform biotinylated AAP10-pTyr show no affinity for Cx43 CT.

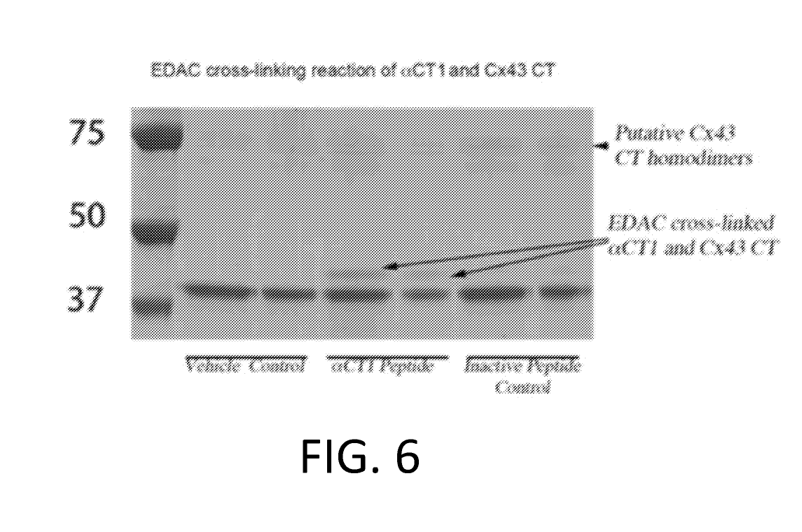

FIG. 6 is an image of a polyacrylamide gel showing EDAC cross-linking reaction of ACT-1 and connectin 43 carboxy terminal peptide. ACT-1, but not inactive control peptide interacts with the Cx43 CT. ACT-1 is shown by the zero order cross-linker EDAC. ACT-1 binding to Cx43 CT reduces Cx43 CT homodimers.

### Example 2

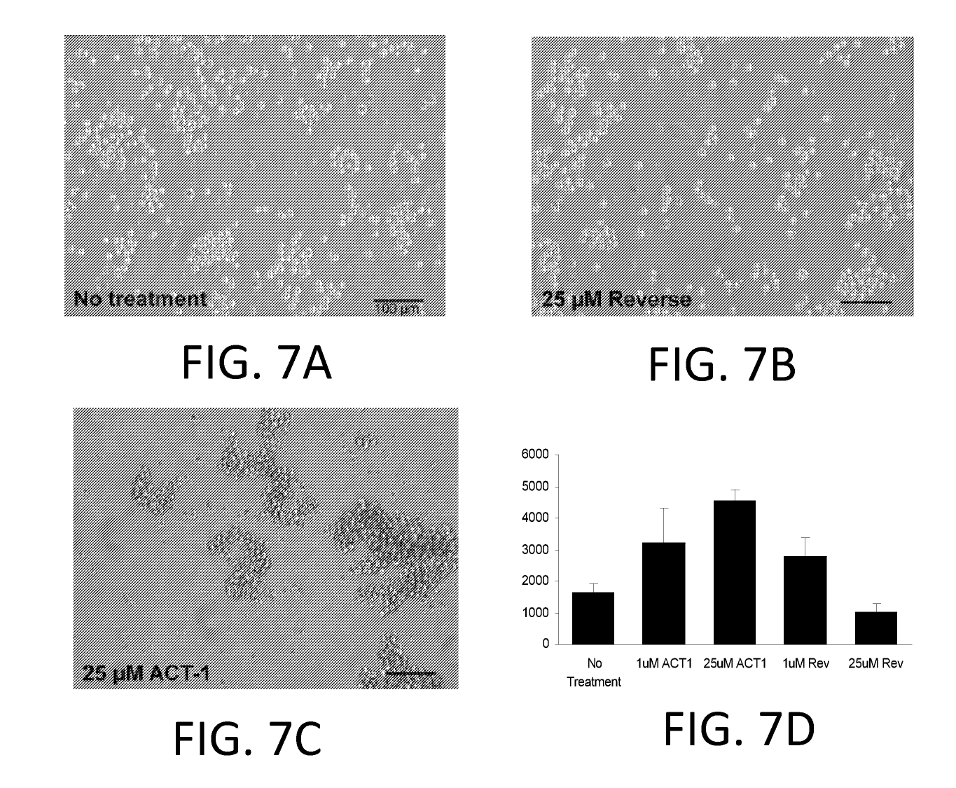

UM87MG glioma cells, which express Cx43, were cultured in serum-free conditions permissive to aggregation. The cells were treated with the ACT-1 peptide for 48 hours and then imaged with phase-contrast microscopy. It can be observed that cells treated with ACT-1 at a concentration of 25 μM tended to form amorphous clumps (FIG. 7C) that were not as prevalent in untreated cultures (FIG. 7A) or in those treated with a control peptide (FIG. 7B), which has the ACT-1 amino acid sequence in reverse order. To quantitate the extent of aggregation, ImageJ software was used to collect size information about the aggregates in these images and from that information an aggregation index was calculated for each image. The AI is defined as the total area of aggregation in a field divided by the number of single, unaggregated cells in that field. So an increase in the ratio of aggregation area to unaggregated cells would increase the AI and indicate a higher propensity to aggregate. The AI calculations for the U87 cells (FIG. 7D, which compares no treatment and four treatments (1 μM ACT-1, 25 μM ACT-1, 1 μM Reverse Peptide, 25 μM Reverse Peptide)) showed that images of cultures treated with 25 μM ACT-1 had significantly higher AIs relative to controls according to the Bonferroni t-test, suggesting that ACT-1 increases the U87 cells' propensity to adhere to each other.

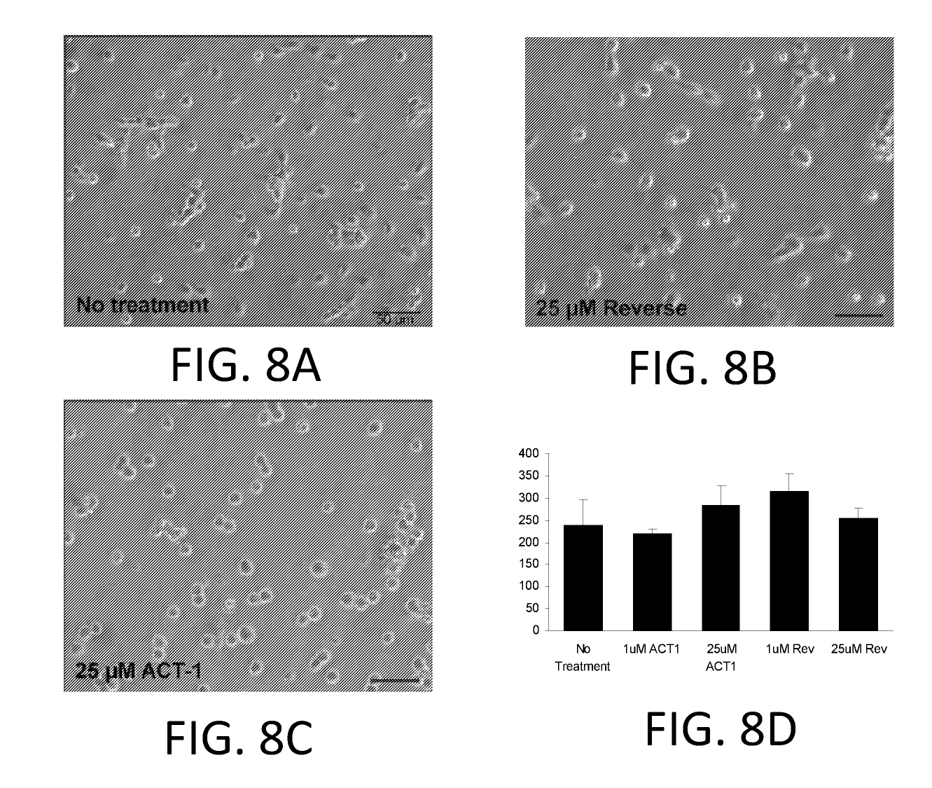

In contrast, when the same assay was performed on Cx-deficient C6 glioma cell line, no significant difference in aggregation between ACT-1 treated cells (FIG. 8C) and controls (no treatment FIG. 8A and reverse (control peptide) treatment FIG. 8B) was observed, as shown in in graph of FIG. 8D, which compares no treatment and four treatments (1 μM ACT-1, 25 μM ACT-1, 1 μM Reverse (Control) Peptide, 25 μM Reverse (Control) Peptide)).

### Example 3

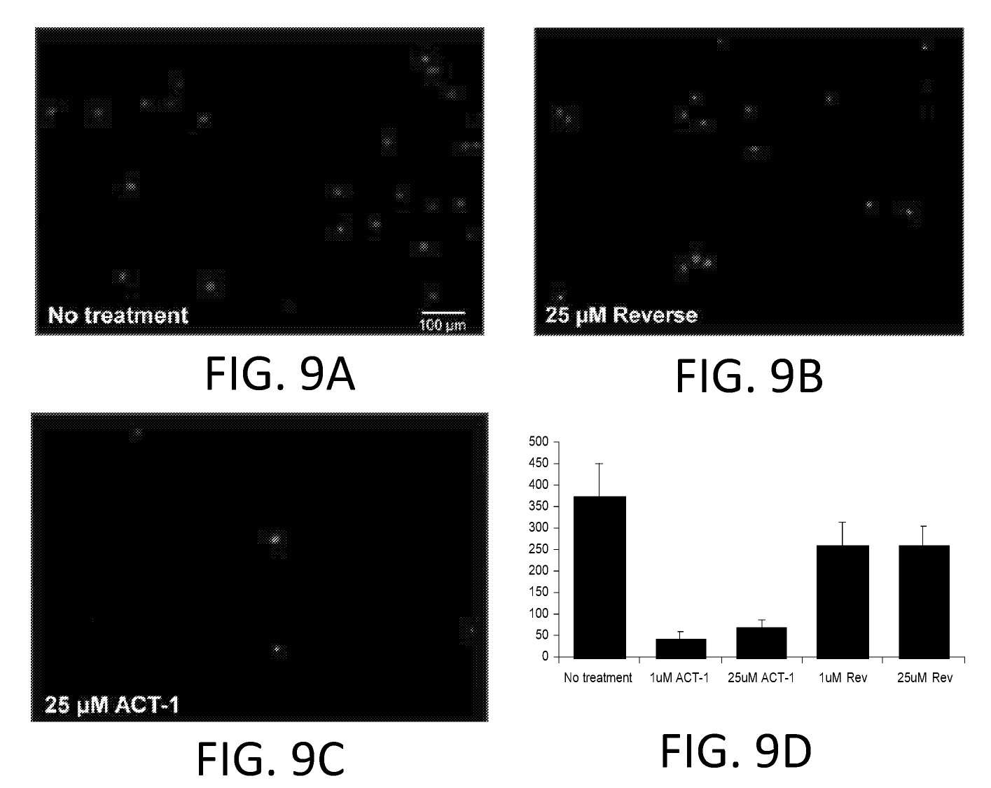

After an overnight peptide pre-treatment in 10% serum, two glioma cell lines, c6 and U87, were seeded in serum-free media containing peptides into Boyden chamber transwell inserts with 8 μm pores in the membrane and then filled the bottom wells with media containing 10% FBS as a chemoattractant. After a 4 hour incubation period, the cells that did not migrate through the membrane were scrapped off. Then the migrated cells were fixed, stained with Hoescht dye, and imaged on an epifluorescence microscope. The nuclei in the images of each treatment group were then counted, and in U87 cultures treated with the ACT-1 peptide (FIG. 9C), there was a significant decrease in cell migration across the Boyden chamber membrane compared to no treatment (FIG. 9A) and control peptide treatment (FIG. 9B), indicating a lesser degree of motility in ACT-1 treated cells (also shown in the graph in FIG. 9D, which compares no treatment and four treatments (1 μM ACT-1, 25 μM ACT-1, 1 μM Reverse (Control) Peptide, 25 μM Reverse (Control) Peptide)).

### Example 4

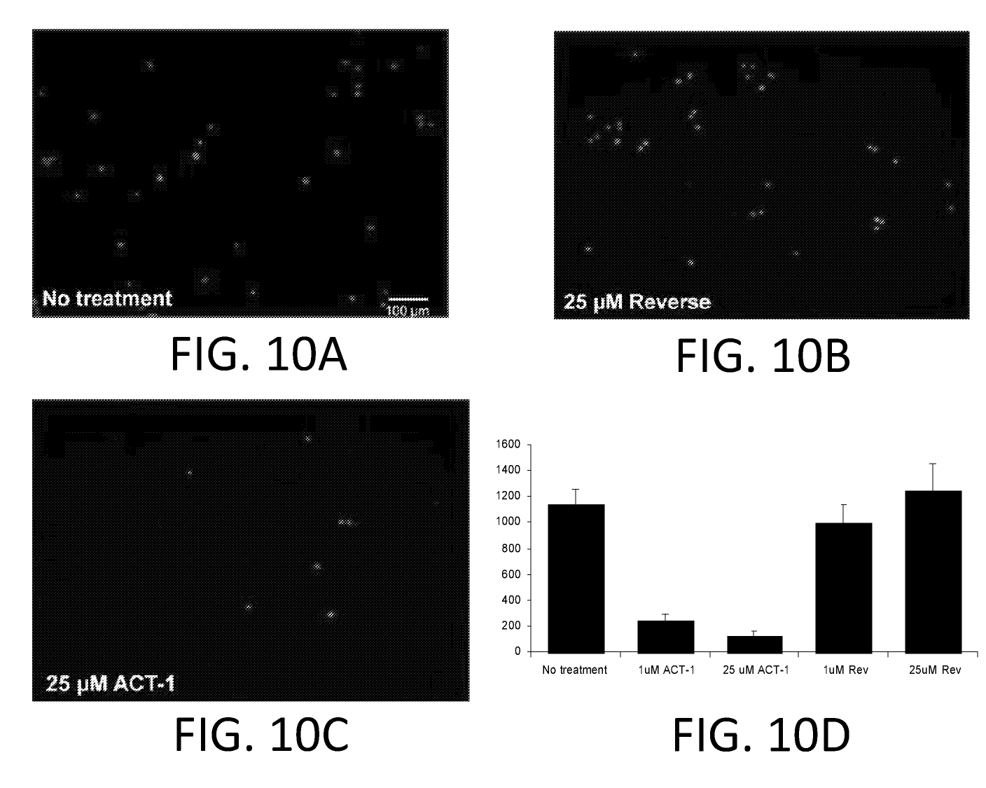

In this example, peptides were added after cells were incubated in serum-free media, then 24 hours of pre-treatment was allowed in the serum-free conditions, and finally the cells were transferred, without typsinizing, to Boyden chamber and incubated for 4 hours. FIG. 10A shows no treatment, FIG. 10B shows reverse (control) peptide treatment, and FIG. 10C shows treatment with 25 μM ACT-1. FIG. 10D shows a graph comparing no treatment and four treatments (1 μM ACT-1, 25 μM ACT-1, 1 μM Reverse (Control) Peptide, 25 μM Reverse (Control) Peptide). The results show that with this method, more of the U87 and C6 cells migrated overall than the method of Example 3, but there was still a 5× decrease in migration in cells treated with both concentrations of ACT-1 (shown in the graph in FIG. 10D). With this example, the results of the C6 cells seemed to be similar to wt-C6 cells.

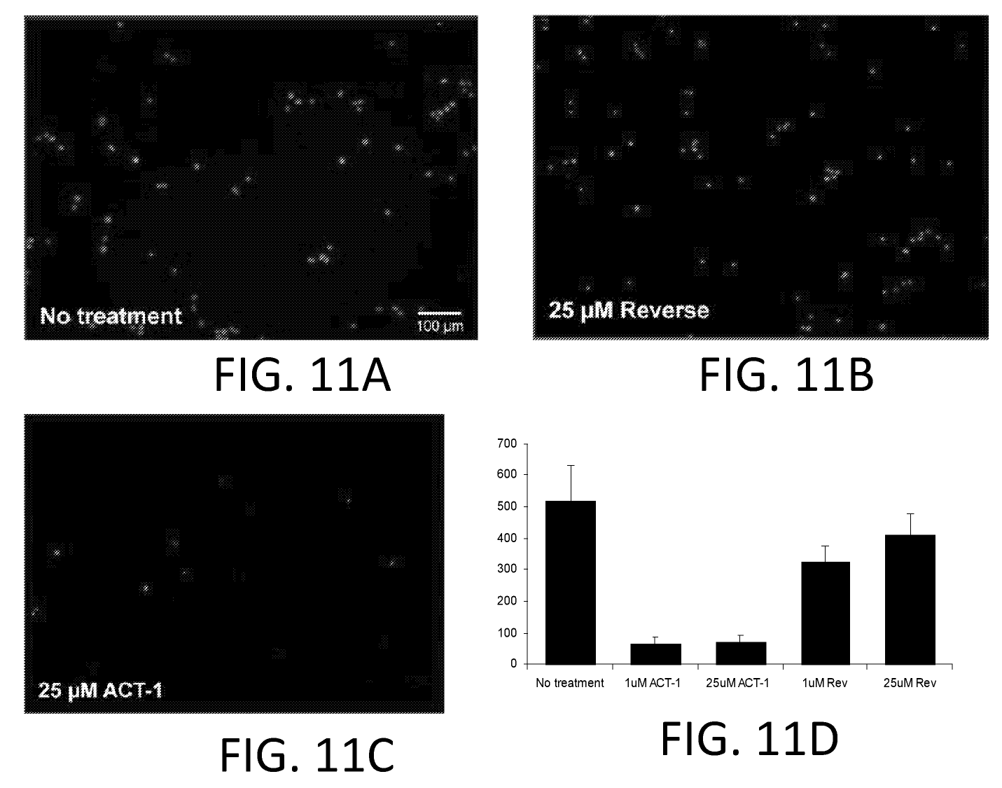

The same situation was true when the same experiment was performed on wild-type C6 cells. FIG. 11A shows no treatment, FIG. 11B shows reverse peptide treatment, and FIG. 11C shows treatment with 25 μM ACT-1. FIG. 11D shows a graph comparing no treatment and four treatments (1 μM ACT-1, 25 μM ACT-1, 1 μM Reverse (Control) Peptide, 25 μM Reverse (Control) Peptide). Both concentrations of ACT-1 seemed to cause about a 5× decrease in motility across the Boyden chamber, but the HA-oligomer treatment did not appear to be different from no oligomer.

### Example 5

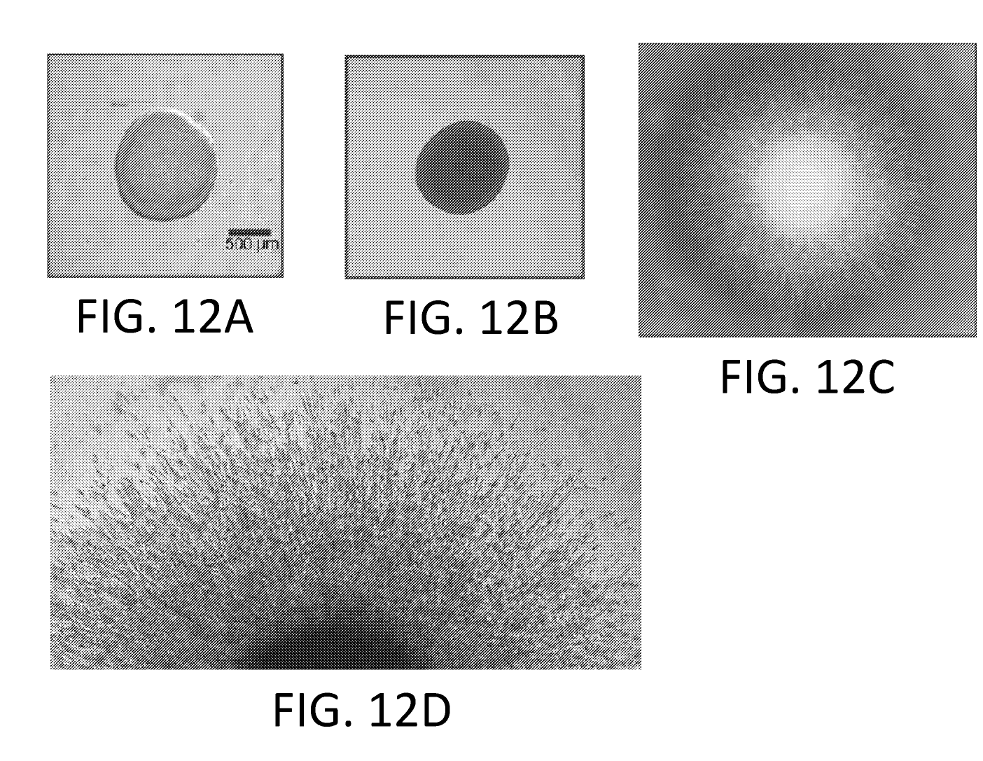

To determine how the ACT-1 peptide will affect protease-dependent glioma cell invasion, a model of glioma invasion into a 3-dimensional collagen gel is used. This model is chosen because a 3-D microenvironment for tumor invasion is one step closer than more classical models of one-dimensional invasion to the actual environment surrounding a tumor mass in vivo. So, glioma spheroids, representative of tumor masses are formed by gravity-driven sedimentation in hanging drops then implanted into a 3-D collagen gel, to which ACT-1 peptide is added before gelation. Shown is a C6 glioma spheroid that can be generated and implanted into collagen gel. FIG. 12A shows a glioma cell aggregate formed by hanging drop sedimentation and FIG. 12B shows a glioma spheroid implanted into 3D collagen gel. It can be seen in FIGS. 12C and 12D that after 48 hours, the cells invade out from the spheroid into the collagen in a sunburst pattern, with web-like projections of cells coming off the central spheroid. This invasion could be quantified by measuring the distance from the center of the spheroid to the farthest invading cell and then normalizing that against the radius of the original spheroid.

### Example 6

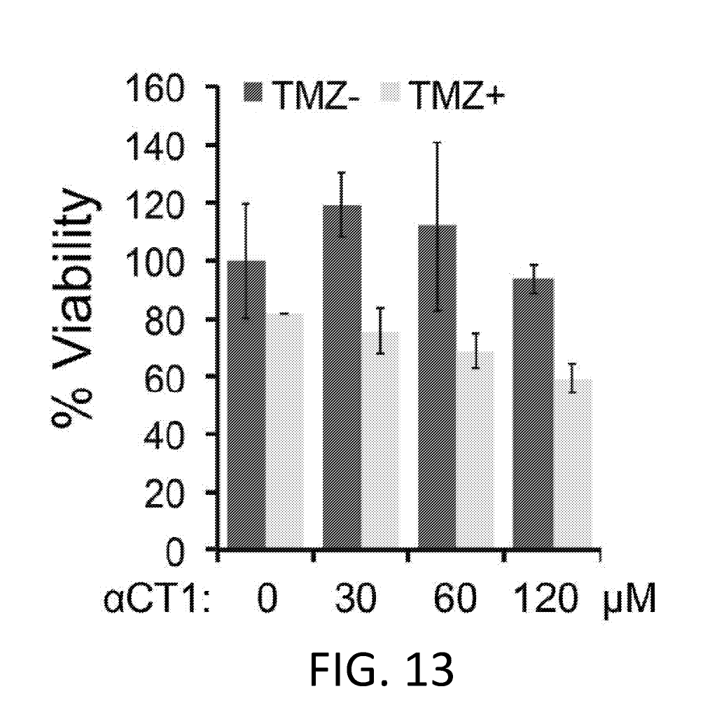

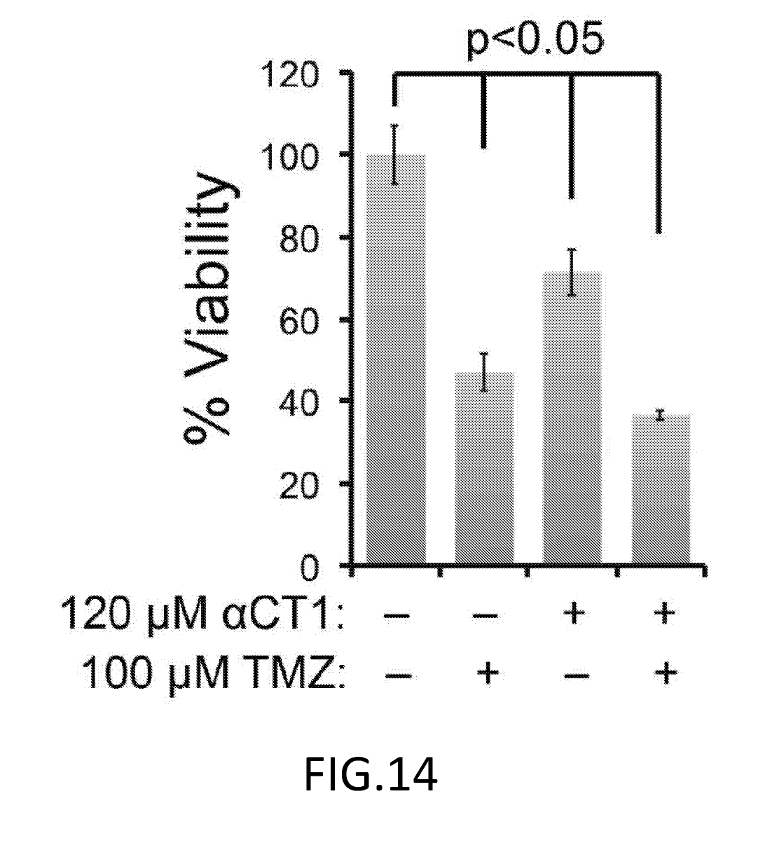

The ACT-1 peptide was tested on a glioma stem cell (GSC) line alone or in combination with temozolomide (TMZ). It was determined that ACT-1 or TMZ alone only mildly reduced the viability of GS9-6 cells, whereas the combination of the two agents significantly inhibited it. The efficacy of the ACT-1-TMZ combinatorial treatment on GSCs improved in a dose-dependent manner with respect to ACT-1 concentration from 30 to 120 μM (FIG. 13). Similarly, the combination of 120 μM ACT-1 and 100 μM TMZ had a greater effect than each of these treatments individually (FIG. 14).

### Example 7

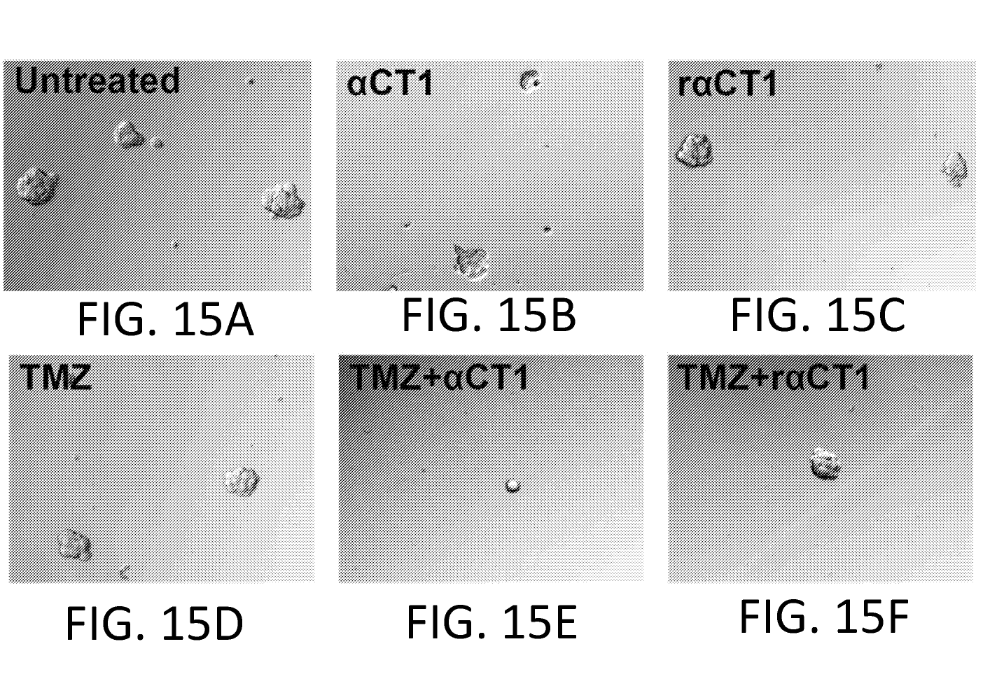

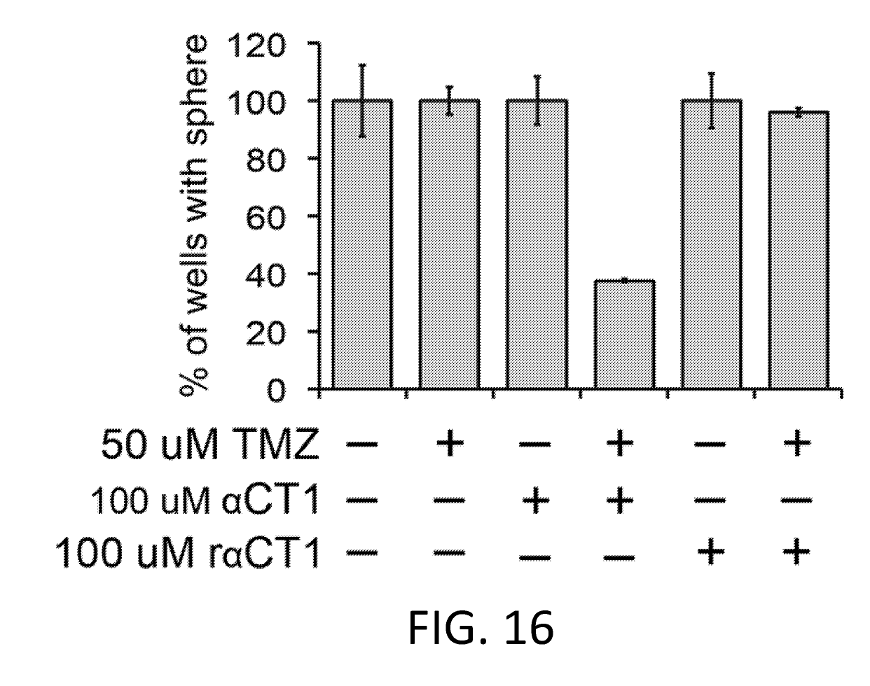

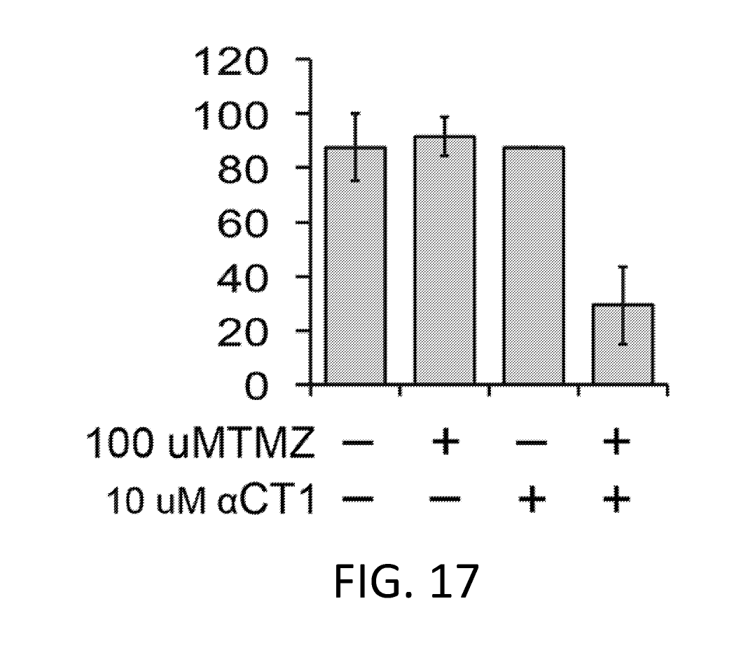

To further investigate the effect of ACT-1 on glioma stem cells, the present inventors measured the capacity of glioma stem cells to self-renew. The unique feature that makes glioma stem cells different from other glioma cells is that glioma stem cells are able to copy themselves (termed as self-renewal). The well-established approach to measure self-renewal of stem cells is sphere/colony formation assay. The self-renewal of GS9-6 glioma stem cells treated with ACT-1 was then monitored. It was found that in untreated cells (FIG. 15A), self-renewal of GS9-6 cells was not affected. Similar results were obtained in cells treated with ACT-1 (FIG. 15B), rACT-1 (FIG. 15C), an inactive ACT-1), TMZ (FIG. 15D), or rACT-1 and TMZ (FIG. 15F) However, the combination of ACT-1 and TMZ substantially abrogated the capability of GS9-6 cells to self-renew (FIG. 15E). Two different doses of ACT-1 and TMZ were tested. Both treatments inhibited the self-renewal of GS9-6 (FIGS. 16 and 17). Thus, the data strongly indicate that ACT-1 sensitizes glioblastoma cells to TMZ.

### Example 8

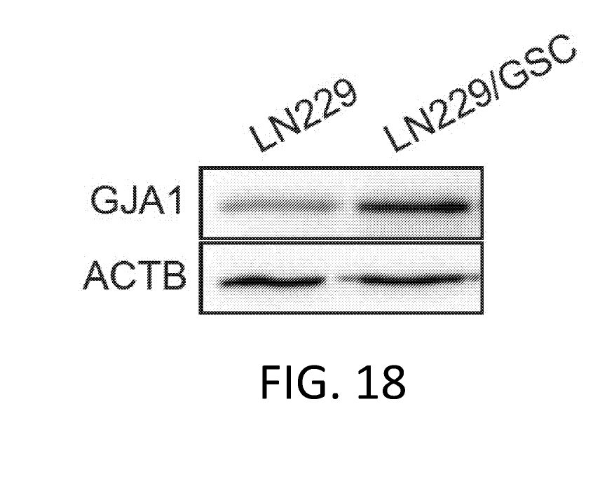

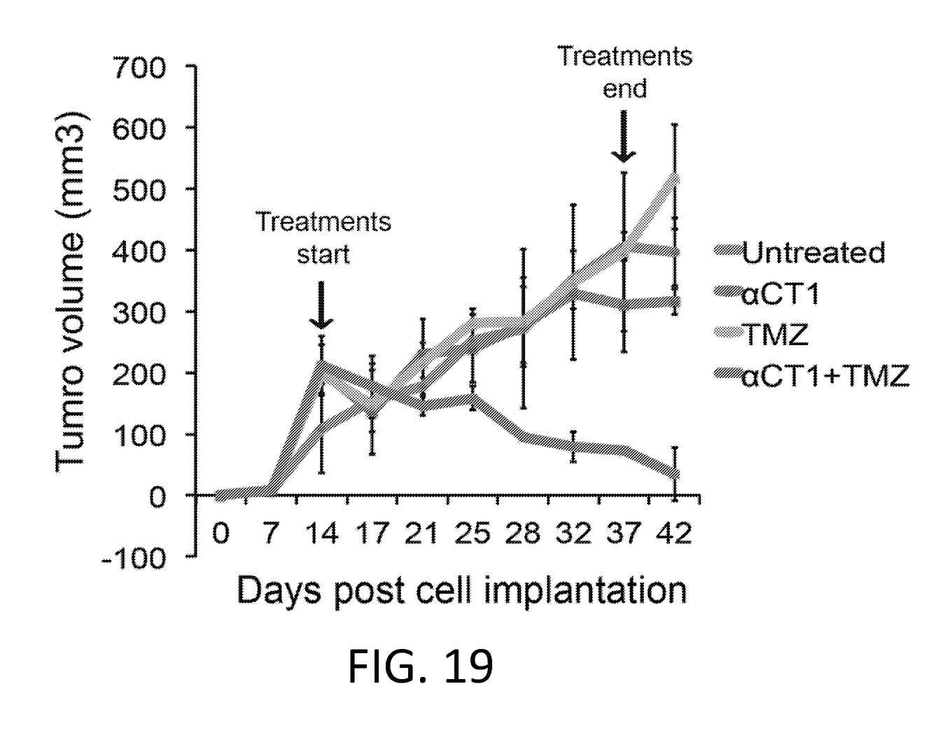

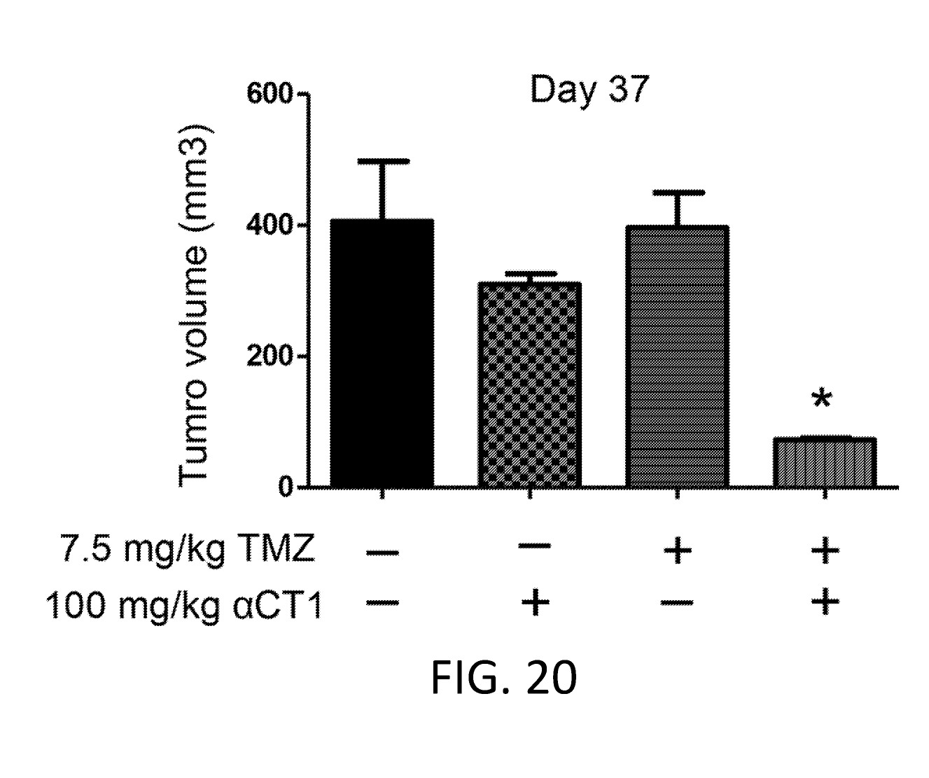

To further validate the therapeutic effect of ACT-1 in vivo, tumor growth was monitored using a glioma xenograft mouse model. As GS9-6 cells grow slowly in mice, the present inventors elected to use LN229/GSCs, isolated from LN229 human GBM cell line. As shown in FIG. 18, connexin 43 (GJA1) was expressed at much higher level in LN229/GSCs compared to parental LN229 cells suggesting that LN229/GSCs may respond to ACT-1 effectively. LN229/GSCs were first injected into the flanks of immunocompromised mice and tumors (more than 100 mm3) were detected in all mice in 14 days. By contrast, GS9-6 cells form a tumor in 6 months (data not shown). Treatments at day 14 were then started. First, 100 mg/kg ACT-1 was injected subcutaneously. 24 hours later, mice were treated with 7.5 mg/kg TMZ (dissolved in DMSO) or vehicle DMSO intraperitoneally. Such treatment regime was repeated three times a week for three weeks, and tumor growth was monitored for another week. FIGS. 19 and 20 show that the combination of ACT-1 and TMZ, but not other treatments, blocked the tumor growth in mice. The data demonstrate that ACT-1 substantially increases the sensitivity of glioblastoma tumors to TMZ. Taken together, these examples provide a firm support for potential clinical application of ACT-1 in treating human or canine glioblastoma patients.

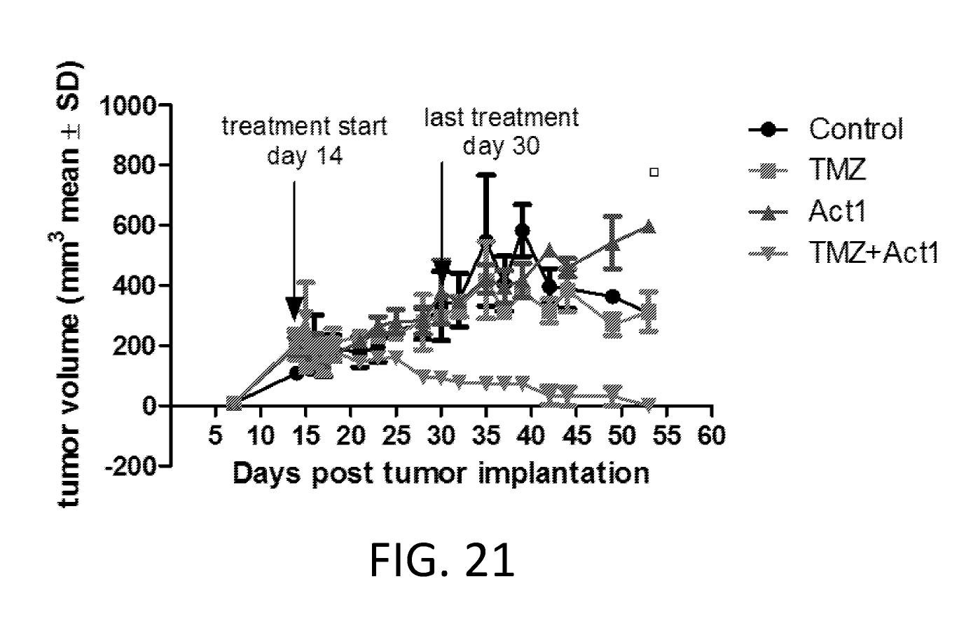

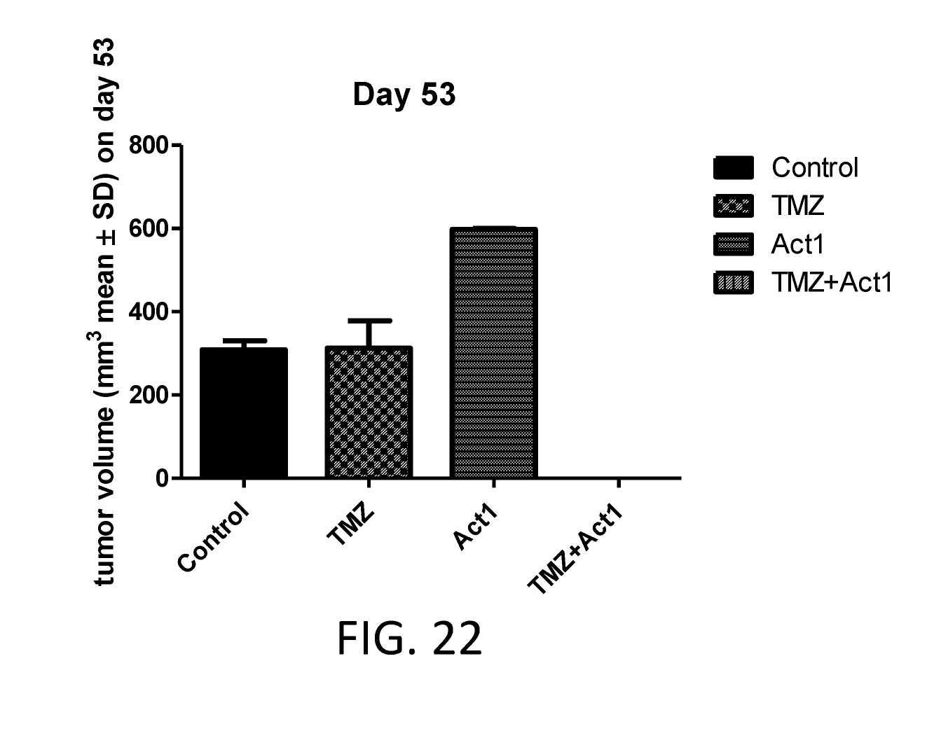

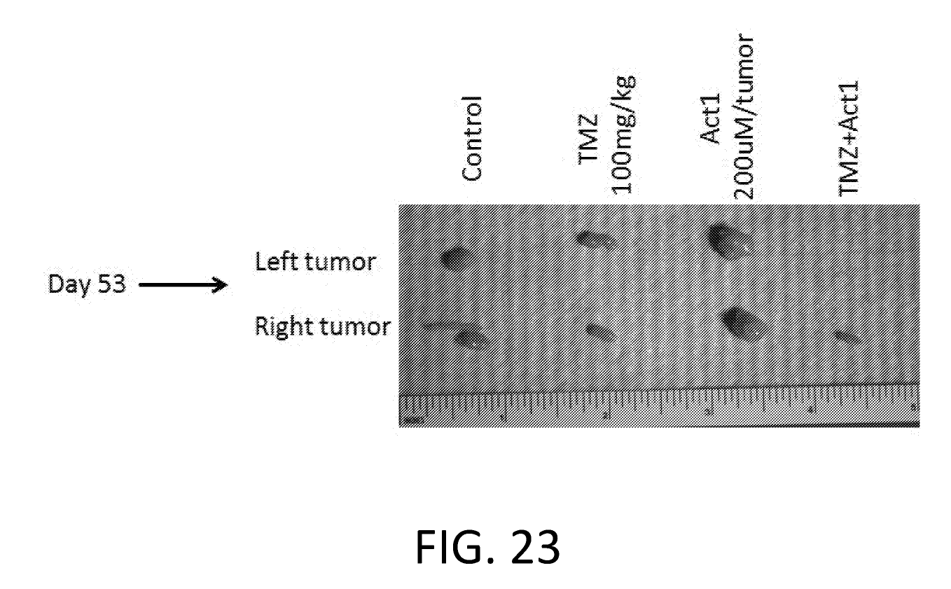

FIG. 21 is a plot of tumor volume over 53 days for untreated, 200 μM/tumor ACT-1, 100 mg/kg TMZ treated, and combinatorial 200 μM/tumor ACT-1 and 100 mg/kg TMZ treated mice. FIG. 22 is a graph of tumor volumes at 53 days and FIG. 23 is an image of tumor volumes at 53 days for these treatments.

As can be seen in FIGS. 21-23, the tumors in the combinatorial ACT-1 and TMZ group withered away to almost nothing. However, the results suggest that ACT-1 when administered by itself (i.e. without TMZ) may promote tumor growth, which was unexpected. The combinatorial treatment also showed unexpected results, as the substantial effect of this treatment in reducing tumor volume could not be predicted from the individual ACT-1 and TMZ treatments, and thus these results were a nonobvious phenomenon of this combination treatment. Not wishing to be bound by theory, sensitivity to TMZ may be propagated in tumors by ACT-1 via Cx43 channels (the so called “bystander effect”).

### Example 9

### Preliminary Studies

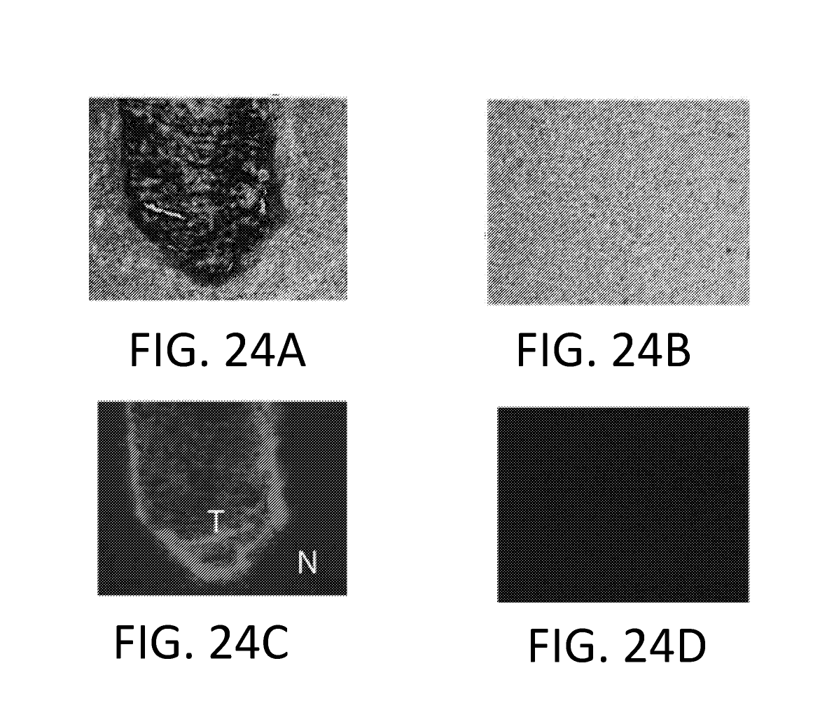

An IL13 peptide Specifically Delivered Nanoparticles to Human GBM Xenografts. In order to specifically deliver therapeutics into tumor cells, which is expected to substantially enhance the efficacy of cancer treatments, IL13-directed tumor targeting was tested in a mouse orthotopic GBM model. IL13 receptor α2 is highly expressed by malignant glioma cell (see Debinski, W., et al., Receptor for interleukin 13 is abundantly and specifically over-expressed in patients with glioblastoma multiforme. International journal of oncology, 1999. 15(3): p. 481-6; see Debinski, W., et al., Receptor for interleukin 13 is a marker and therapeutic target for human high-grade gliomas. Clinical cancer research: an official journal of the American Association for Cancer Research, 1999. 5(5): p. 985-90). An IL13 peptide-based delivery crossed the BBB and homed therapeutic agents to GBM in mice (see Pandya, H., et al., An interleukin 13 receptor alpha 2-specific peptide homes to human Glioblastoma multiforme xenografts. Neuro-oncology, 2012. 14(1): p. 6-18.). More importantly, IL13-directed tumor specific delivery of therapeutics has been recently evaluated in a phase I clinical trial for brain cancer patients and no significant adverse effects were observed (see Kunwar, S., et al., Direct intracerebral delivery of cintredekin besudotox (IL13-PE38QQR) in recurrent malignant glioma: a report by the Cintredekin Besudotox Intraparenchymal Study Group. Journal of clinical oncology: official journal of the American Society of Clinical Oncology, 2007. 25(7): p. 837-44). To test whether this tumor-specific delivery system works in our hands, we intravenously injected nanoparticles coupled with a TAMRA-conjugated IL13 peptide into mice harboring a human U251 orthotopic xenograft GBM. IL13 peptide-containing nanoparticles indicated by TAMRA red fluorescence were found in U251 orthotopic xenografts, but not in the normal brain contralateral to the tumor. FIG. 24A shows a bright field view of U251 xenograft and FIG. 24B shows a bright field view of contralateral brain, while FIG. 24C shows TAMRA imaging of U251 xenograph and FIG. 24D shows TAMRA imaging of contralateral brain. These data suggest that IL13 peptide-conjugated nanoparticles crossed the BBB and specifically targeted GBM tumors.

### Example 10

Rationale: The half-life of ACT-1 in the body is less than a few hours. This poses a technical issue if ACT-1 activity in the brain is to be sustained during TMZ treatment of glioblastoma. To meet this challenge an ACT-1-expressing viral-based vector is engineered that will provide continuous secretion of ACT-1 in the GBM tumor microenvironment. The construct is built on an adeno-associated virus (AAV) backbone. AAVs are able to provide long-term expression of new genes in human cells at high efficiency without causing genetic mutation or strong immune reactions. Because of these properties AAVs are gaining acceptance as the vector of choice for gene therapy in humans.

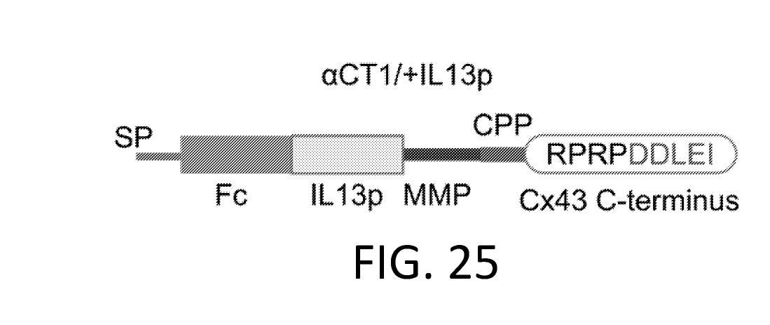

Approach: To ensure maximum sustained expression vectors will be used that are built upon an AAV backbone that permits direct transduction of cells in the tumor microenvironment. Production of a chimeric protein termed ACT-1/+IL13P is generated via transcription from a strong CMV promoter. The rationale and structure of this chimeric protein is shown in FIG. 25 and is described as follows: At its N-terminus it will have a signal peptide (SP), enabling efficient secretion of the chimeric protein from cells. A human Fc fragment of immunoglobulin will stabilize the chimeric protein during translation and secretion16. IL13 receptors are strongly expressed in GBM tumors, thus the construct will include an Il13 peptide that will localize the chimeric protein at GBM cells. An MMP peptidase cleavage motif will release active ACT-1, enabling transduction of all cells local to secreting cells via its cell penetration peptide (CPP) Another selection is added by placing EGFP genes under the control of an internal ribosomal entry site. The resulting tumor-targeting plasmid pAAV-ACT-1/+IL13P is used to make infectious AAV-ACT-1/+IL13P particles. To test the construct in vitro, HEK293 cells are transduced with the virus. Expression of active ACT-1 is confirmed by Western blotting. Whether the secreted protein interacts with the PDZ2 domain of ZO-1—its molecular target-in a dose-dependent manner is evaluated. Finally, the functional activity of ACT-1 using assays of gap junction coupling and Cx43 hemichannel activity-routine biological assays of ACT-1 activity is assessed.

To gauge the therapeutic response of glioblastoma cells to the ACT-1-expressing virus and TMZ in vivo, it will be tested in a human orthotopic GBM mouse model that has been developed and validated by Dr. Sheng and his lab. Human GS9-6 GSCs are first transplanted into the brain of immunocompromised mice. After tumor cells start to grow (1-2 months), virus is injected intracranially in the region where GS9-6 cells are implanted. Mice are then treated with TMZ (7.5 mg/kg). Appropriate controls, including use of viruses not expressing chimeric protein are carried out in parallel. In vivo activity is determined by: 1) Monitoring tumor progression in live animals using magnetic resonance imaging (MRI); 2) Scoring survival of GBM mice using Kaplan Meier survival assays and 3) Histology of brain tissue.

Safety assessments are undertaken in normal mouse injected intracranially with AAV-ACT-1/+IL13P, but with no accompanying glioma cells. These mice will be scored using Kaplan Meier survival assays and major organs—heart, lungs, live and kidneys-will be subject to gross and histological analyses at 6 months and 1 year for abnormalities and disease by a qualified veterinary pathologist at the VTCRI. Rates of reproduction and offspring birth defects are monitored in experimental animals to screen for abnormalities in breeding and effects on embryos that are carried by treated animals.

i) To Engineer rAAV Vectors that Express Chimeric ACT-1 Proteins.

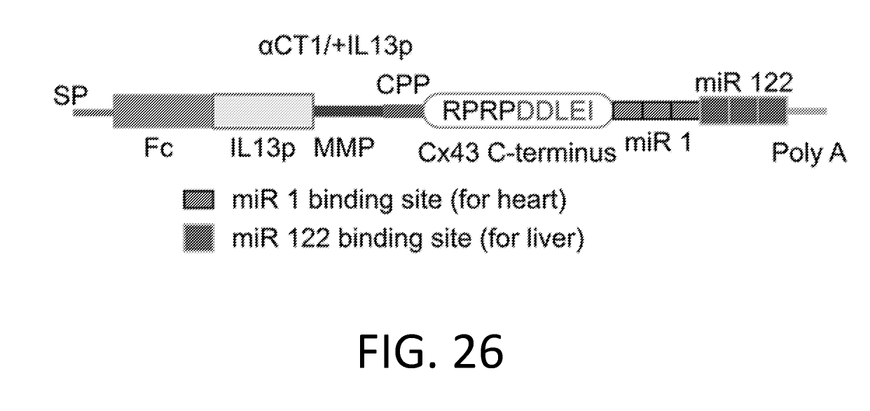

To ensure the maximum expression of transgene in the brain, the present inventors elect to use the vector AAV.rh10 that has been validated in a previous report (see Zhang, H., et al., Several rAAV vectors efficiently cross the blood-brain barrier and transduce neurons and astrocytes in the neonatal mouse central nervous system. Molecular therapy: the journal of the American Society of Gene Therapy, 2011. 19(8): p. 1440-8). This vector harbors a hybrid promoter cassette composed of cytomegalovirus enhancer and a minimal beta-actin promoter that will drive expression of transgene in most cells and tissues. Whilst IL13 peptide will deliver this chimeric protein to the GBM tumor, the overexpression of ACT-1 in other organs (i.e. heart or liver) may potentially cause damage. AAV vectors often accumulate in the liver (see Xie, J., et al., MicroRNA-regulated, systemically delivered rAAV9: a step closer to CNS-restricted transgene expression. Molecular therapy: the journal of the American Society of Gene Therapy, 2011. 19(3): p. 526-35) and gap junction protein Cx43 plays a critical role in the heart (see Palatinus, J. A., J. M. Rhett, and R. G. Gourdie, The connexin43 carboxyl terminus and cardiac gap junction organization. Biochimica et biophysica acta, 2012. 1818(8): p. 1831-43). Thus, minimizing the expression of ACT-1 in these two organs could significantly reduce potential damage caused by non-specific ACT-1 overexpression. The present inventors will utilize the micro-RNA-based strategy developed by the inventors' collaborator Dr. Gao. As shown in FIG. 26, multiple miR1 and miR-122 binding sites will be placed at the 3′ end of the DNA fragment that encodes the chimeric ACT-1. miR1 or miR122 suppresses the expression of transgene in the heart or liver, respectively (see. Xie, J., et al., MicroRNA-regulated, systemically delivered rAAV9: a step closer to CNS-restricted transgene expression. Molecular therapy: the journal of the American Society of Gene Therapy, 2011. 19(3): p. 526-35).

A control AAV vector that expresses a chimeric ACT-1 without IL13 peptide (referred to as ACT-1/−IL13p) will be made, in contrast to the functional chimeric ACT-1 (termed ACT-1/+IL13p). Based on the results shown in FIGS. 25A-25D, it is expected that ACT-1/−IL13p will fail to sensitize GBM cells to TMZ due to the lack of tumor-microenvironment specific targeting. All the vectors will be produced using transient transfection in HEK293 (human embryonic kidney) cells followed by the CsCl gradient sedimentation.

ii) To Determine the Expression and Activity of Chimeric ACT-1 Proteins in Vitro.

To test whether the chimeric ACT-1 proteins can be expressed and secreted, HEK293 cells will be transiently infected with the above AAV vectors. The expression of ACT-1s in cell lysates and its secretion to the culture media will be evaluated using an antibody that recognizes Cx43 CT and has been validated by the Gourdie group (see O'Quinn, M. P., et al., A peptide mimetic of the connexin43 carboxyl terminus reduces gap junction remodeling and induced arrhythmia following ventricular injury. Circulation research, 2011. 108(6): p. 704-15). The present inventors expect to detect the full-length chimeric protein (˜60-70 kDa) in both cell lysates and culture media and/or, if any, the cleaved CPP-ACT-1 (˜3-5 kDa) in cell lysates only.

Next, it can be determined whether ACT-1/+IL13p is functional using the dye coupling assay (monitoring the activity of gap junctions) and ZO-1 interaction assays (detecting interaction between Cx43 and ZO-1). HEK293 cells are transduced with the AAV vectors and then culture media that contain secreted ACT-1/+IL13p or ACT-1/−IL13p are transferred to U87MG cells. The fluorescent dye Calcein AM (Molecular Probes) transfers through gap junctions between adjacent cells. It is expected that ACT-1/+IL13p, but not ACT-1/−IL13p, will alter the dye coupling between adjacent U87MG cells. Co-immunoprecipitation assay is also performed using antibodies against Cx43 or ZO-1. ACT-1/+IL13p is expected to be co-immunoprecipitated with ZO-1 in U87MG cells, whereas ACT-1/−IL13p will fail due to the lack of tumor-specific targeting.

iii) To Measure the Expression Level and Therapeutic Effect of Chimeric ACT-1 Proteins In Vivo.

To test whether intravenously injected AAV.rh10 express chimeric ACT-1 proteins in the mouse brain. 4×10¹² genome copies of the rAAV vectors (empty, ACT-1/+IL13p, or ACT-1/−IL13p) is injected into the immunocompromised mice through the tail vein. 3-4 weeks later, expression of the chimeric ACT-1 proteins in the brain, liver, and heart is detected using IHC. A horseradish peroxidase-conjugated antibody that recognizes immunoglobulin G Fc fragment (Abcam, Cambridge Mass.) is used in IHC assays. This antibody will detect both ACT-1/+IL13p and ACT-1/−IL13p as they contain Fc fragment but will not recognize endogenous Cx43. It is expected that the Fc antibody staining will be higher in the brain expressing chimeric ACT-1 proteins ACT-1/+IL13p or ACT-1/−IL13p, but not the empty vector. No or limited expression of chimeric ACT-1 proteins is expected in heart and liver due to the micro-RNA-based suppression. The in vivo activity of ACT-1/+IL13p or ACT-1/−IL13p is determined by the assays described in section C1.2.11 It is expected that in vivo TMZ sensitivity will be substantially increased by ACT-1/+IL13p, but not ACT-1/−IL13p.

### Example 11

Spatially control delivery of active ACT-1 to the mouse brain using bone marrow-derived MSCs can be achieved based on the following reasons: 1) MSCs have a strong tumor tropism, which allows MSCs to bypass the BBB (see Bexell, D., Svensson, A. & Bengzon, J. Stem cell-based therapy for malignant glioma. Cancer Treat Rev 39, 358-365 (2013)) and enhance tumor-specific targeting. Interferon β or TRAIL has been successfully delivered to the mouse brain using these cells. (see Nakamizo, A., et al. Human bone marrow-derived mesenchymal stem cells in the treatment of gliomas. Cancer Res 65, 3307-3318 (2005); Menon, L. G., et al. Human bone marrow-derived mesenchymal stromal cells expressing S-TRAIL as a cellular delivery vehicle for human glioma therapy. Stem Cells 27, 2320-2330 (2009)) 2) MSCs are readily acquired from patient bone marrow and are expandable. 3) MSCs can be transduced efficiently by lenti-viruses (see Aguilar, S., et al. Bone marrow stem cells expressing keratinocyte growth factor via an inducible lentivirus protects against bleomycin-induced pulmonary fibrosis. PLoS One 4, e8013 (2009)). 4) MSCs are an immune-privileged lineage thus limiting complications. One of the inventors has determined that ACT-1 has effects cell migration that may affect MSC-targeting. To circumvent this the inventors will use a Tet-On expression system to ensure temporal control of ACT-1 delivery only when MSCs have reached their targets. Tet-On has strong advantages over other inducible gene switches: 1) Tet is one of the longest established systems and thus has undergone considerable iteration and re-engineering over the years, including near abolition of leaky expression; 2) Doxycycline (Dox), a derivative of tetracycline, can be taken orally and has excellent brain penetration (CSF to serum ratio is ˜0.26/26%); 3) The dose-response correlation of gene expression to varying Dox dosage is precise; 4) The Tet-On system has proven to give minimal immune response in animals, suitable for human use; 5) To date, only the Tet-On system has been applied in large animal models such as primates; 6) Other systems such as mifepristone-inducible system are not suitable for GBM as mifepristone itself sensitizes GBM cells to TMZ, thereby complicating results (see Stieger, K., Belbellaa, B., Le Guiner, C., Moullier, P. & Rolling, F. In vivo gene regulation using tetracycline-regulatable systems. Adv Drug Deliv Rev 61, 527-541 (2009); Agwuh, K. N. & MacGowan, A. Pharmacokinetics and pharmacodynamics of the tetracyclines including glycylcyclines. J Antimicrob Chemother 58, 256-265 (2006); Favre, D., et al. Lack of an immune response against the tetracycline-dependent transactivator correlates with long-term doxycycline-regulated transgene expression in nonhuman primates after intramuscular injection of recombinant adeno-associated virus. J Virol 76, 11605-11611 (2002); Latta-Mahieu, M., et al. Gene transfer of a chimeric trans-activator is immunogenic and results in short-lived transgene expression. Hum Gene Ther 13, 1611-1620 (2002); Chenuaud, P., et al. Optimal design of a single recombinant adeno-associated virus derived from serotypes 1 and 2 to achieve more tightly regulated transgene expression from nonhuman primate muscle. Mol Ther 9, 410-418 (2004); Aurisicchio, L., et al. Regulated and liver-specific tamarin alpha interferon gene delivery by a helper-dependent adenoviral vector. J Virol 79, 6772-6780 (2005); Stieger, K., et al. Long-term doxycycline-regulated transgene expression in the retina of nonhuman primates following subretinal injection of recombinant AAV vectors. Mol Ther 13, 967-975 (2006); Stieger, K., et al. Oral administration of doxycycline allows tight control of transgene expression: a key step towards gene therapy of retinal diseases. Gene Ther 14, 1668-1673 (2007); Yim, C. W., Flynn, N. M. & Fitzgerald, F. T. Penetration of oral doxycycline into the cerebrospinal fluid of patients with latent or neurosyphilis. Antimicrob Agents Chemother 28, 347-348 (1985); Llaguno-Munive, M., Medina, L. A., Jurado, R., Romero-Pina, M. & Garcia-Lopez, P. Mifepristone improves chemo-radiation response in glioblastoma xenografts. Cancer Cell Int 13, 29 (2013)) and 7) Other systems are not readily available owing to proprietary constraints.

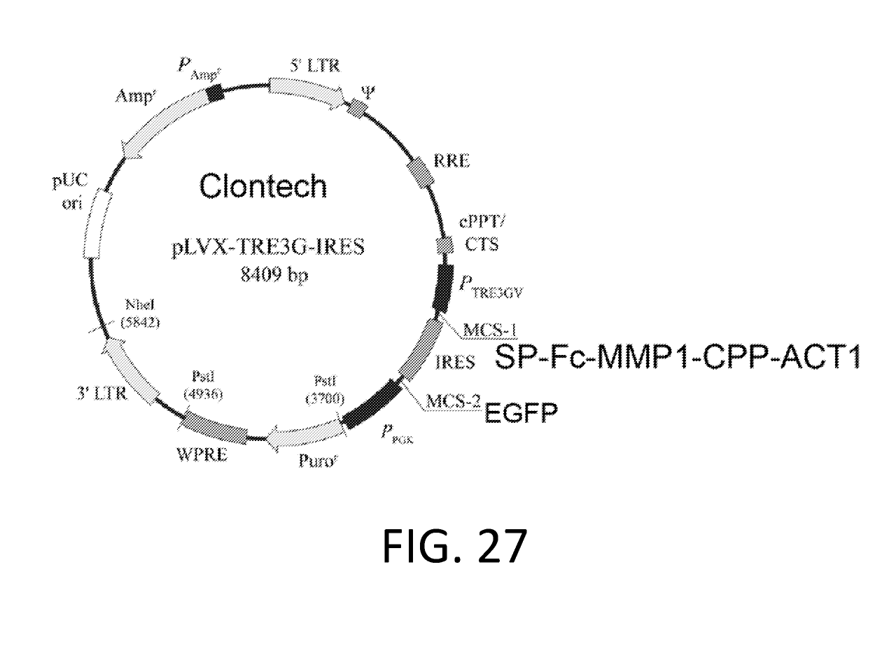

To establish an MSC line expressing an inducible and active ACT-1: i) To engineer an ACT-1 inducible Tet-On expression vector. To ensure the maximum induction and minimal leaky expression, the newest version of the Tet-On system Lenti-X™ Tet-On® developed by Clontech Laboratories, Inc. (Mountain View, Calif.) is purchased. This new system, shown in FIG. 27, uses a TRE3G promoter that provides the tightest control from tetracycline or Dox (see Loew, R., Heinz, N., Hampf, M., Bujard, H. & Gossen, M. Improved Tet-responsive promoters with minimized background expression. BMC Biotechnol 10, 81 (2010)) as well as a most sensitive Tet 3G transactivator (see Urlinger, S., et al. Exploring the sequence space for tetracycline-dependent transcriptional activators: novel mutations yield expanded range and sensitivity. Proc Natl Acad Sci USA 97, 7963-7968 (2000)). FIG. 27 shows that the chimeric ACT1 gene will be inserted into MCS-1 site and EGFP gene will be inserted into the MCS-2 site. The vectors are built upon a lenti-viral backbone (see Zhou, X., Vink, M., Klaver, B., Berkhout, B. & Das, A. T. Optimization of the Tet-On system for regulated gene expression through viral evolution. Gene Ther 13, 1382-1390 (2006)) thereby permitting a high transduction of MSCs. In order to express an active inducible ACT-1 (referred to as iACT-1 hereafter), a chimeric protein termed SP-CPP-Fc-IL-13-MMP1-iACT-1 is made (see Koutsokeras, A., et al. Generation of an efficiently secreted, cell penetrating NF-kappaB inhibitor. Faseb J 28, 373-381 (2014); Lo, K. M., et al. High level expression and secretion of Fc-X fusion proteins in mammalian cells. Protein Eng 11, 495-500 (1998); Shen, Y., Yu, W., Hay, J. G. & Sauthoff, H. Expressed cell-penetrating peptides can induce a bystander effect, but passage through the secretory pathway reduces protein transduction activity. Mol Ther 19, 903-912 (2011); Pandya, H., Gibo, D. M., Garg, S., Kridel, S. & Debinski, W. An interleukin 13 receptor alpha 2-specific peptide homes to human Glioblastoma multiforme xenografts. Neuro Oncol 14, 6-18 (2012); Debinski, W., Gibo, D. M., Slagle, B., Powers, S. K. & Gillespie, G. Y. Receptor for interleukin 13 is abundantly and specifically over-expressed in patients with glioblastoma multiforme. Int J Oncol 15, 481-486 (1999)). The signal peptide (SP) is at the N-terminus of iACT-1. The cell penetrating peptide (CPP) drives iACT-1 internalization into targeted cells. A human Fc fragment of immunoglobin stabilizes iACT-1. IL-13 ensures tumor-specific targeting. An MMP cleavage motif releases active iACT-1 in tumor cells. The generic structure of the protein is shown in FIG. 24. Another selection is added by placing EGFP genes under the control of an internal ribosomal entry site. The resulting tumor-targeting plasmid pLVX-i/+IL-13 and a control plasmid pLVX-i/−IL-13 (that encodes ACT-1, but lacks tumor-cell specificity) are used to make lenti-viruses.

ii) To Make MSC Cell Lines Expressing i/+IL-13 or i/−IL-13.

A mouse MSC line is purchased from Life Technologies Corporation and maintained/expanded in vitro. MSCs are transduced simultaneously with lenti-viruses harboring pLVX-TRE3G and pLVX-i/+IL-13 or pLVX-i/−IL-13, followed by a triple selection of G418 (for transactivator), puromycin (for iACT-1 s), and EGFP-based cell sorting. Expression and secretion of active iACT-1 is confirmed by the following assays. First, the secreted iACT-1 in culture media using a Cx43 CT-recognizing antibody (see O'Quinn, M. P., Palatinus, J. A., Harris, B. S., Hewett, K. W. & Gourdie, R. G. A peptide mimetic of the connexin43 carboxyl terminus reduces gap junction remodeling and induced arrhythmia following ventricular injury. Circ Res 108, 704-715 (2011)) is detected. A robust Dox-induced expression and secretion of iACT-1 in established MSC lines is expected. Next, whether the secreted protein interacts with the PDZ2 domain of ZO-1—its molecular target is determined. The activity of iACT-1 is measured using dye-coupling assays (see Gielen, P. R., et al. Connexin43 confers Temozolomide resistance in human glioma cells by modulating the mitochondrial apoptosis pathway. Neuropharmacology 75, 539-548 (2013)). The fluorescent dye Calcein AM (Molecular Probes) transfers through gap junctions between adjacent cells. Calcein fluorescence in adjacent GBM cells receiving MSC-secreted iACT-1s is measured. It is expected that i/+IL-13, but not i/−IL-13, will alter the dye coupling between adjacent GBM cells.

To determine the therapeutic effect of i-ACT-1 expressing MSCs in vivo: To gauge the therapeutic response of GBM cells to iACT-1 and TMZ, iACT-1-expressing MSCs are tested in a human orthotopic GBM mouse model. Human GS9-6 GSCs are first transplanted into the brain of immunocompromised mice. After tumor cells start to grow (usually 1-2 months), iACT-1-expressing MSCs are injected intracranially near the region where GS9-6 cells were originally implanted. Mice are then fed with Dox-supplemented food ad libitum (200 mg/kg) (see Latta-Mahieu, M., et al. Gene transfer of a chimeric trans-activator is immunogenic and results in short-lived transgene expression. Hum Gene Ther 13, 1611-1620 (2002)) followed by TMZ treatment (7.5 mg/kg) (see Sheng, Z., et al. A genome-wide RNA interference screen reveals an essential CREB3L2-ATF5-MCL1 survival pathway in malignant glioma with therapeutic implications. Nat Med 16, 671-677 (2010)). The in vivo activity of iACT-1 is determined by: 1) Monitoring tumor progression in live animals using magnetic resonance imaging (MRI); 2) Scoring the survival of GBM mice using Kaplan Meier survival assays and 3) Histological analyses of brain. It is expected that in vivo TMZ sensitivity will be substantially increased by i/+IL-13, but not i/−IL-13.

The present invention is described with reference to particular embodiments having various features. In light of this disclosure, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. Other embodiments will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A method of treating or preventing a cancer in a subject, comprising administering to the subject an effective amount of a composition comprising:
a peptide comprising a contiguous sequence of amino acids representing a portion of the carboxy terminus of an alpha connexin protein or conservative variant thereof, wherein said carboxy terminus includes the sequence up to the transmembrane domain, or
a vector comprising a nucleic acid sequence encoding the peptide, or
a host cell comprising the vector,
wherein the peptide does not comprise the full length alpha connexin protein.

2-3. (canceled)

4. The method of claim 1, further comprising administration of a cancer therapeutic agent.

5. The method of claim 1, wherein the cancer is selected from one or more of the group consisting of high-grade astrocytoma, breast cancer, colon cancer, rectal cancer, endometrial cancer, cervical cancer, kidney cancer, leukemia, liver cancer, stomach cancer, esophageal cancer, oral cancer, throat cancer, tracheal cancer, lung cancer, melanoma, non-melanoma skin cancers, non-Hodgkin lymphoma, Hodgkin lymphoma, pancreatic cancer, prostate cancer, head and neck cancers, bone cancer, and thyroid cancer.

6. The method of claim 1, wherein the cancer is a glioma.

7. The method of claim 4, wherein the cancer therapeutic agent is selected from one or more of the group consisting of abiraterone acetate, methotrexate, paclitaxel albumin-stabilized nanoparticle formulation, brentuximab vedotin, ado-trastuzumab emtansine, doxorubicin hydrochloride, fluorouracil, afatinib dimaleate, everolimus, imiquimod, aldesleukin, alemtuzumab, pemetrexed disodium, palonosetron hydrochloride, chlorambucil, aminolevulinic acid, anastrozole, aprepitant, pamidronate disodium, anastrozole, exemestane, nelarabine, arsenic trioxide, ofatumumab, asparaginase erwinia chrysanthemi, bevacizumab, axitinib, azacitidine, bendamustine hydrochloride, bevacizumab, bexarotene, tositumomab and i 131 iodine tositumomab, bleomycin, bortezomib, bosutinib, cabazitaxel, cabozantinib-s-malate, alemtuzumab, irinotecan hydrochloride, capecitabine, carboplatin, carfilzomib, lomustine, daunorubicin hydrochloride, cetuximab, chlorambucil, cisplatin, cyclophosphamide, clofarabine, cabozantinib-s-malate, dactinomycin, crizotinib, ifosfamide, cytarabine, dabrafenib, dacarbazine, decitabine, dactinomycin, dasatinib, daunorubicin hydrochloride, decitabine, degarelix, denileukin diftitox, denosumab, dexrazoxane hydrochloride, docetaxel, doxorubicin hydrochloride, fluorouracil, rasburicase, epirubicin hydrochloride, oxaliplatin, eltrombopag olamine, aprepitant, enzalutamide, epirubicin hydrochloride, cetuximab, eribulin mesylate, vismodegib, erlotinib hydrochloride, etoposide, everolimus, raloxifene hydrochloride, exemestane, toremifene, fulvestrant, letrozole, filgrastim, fludarabine phosphate, fluorouracil, folinic acid, pralatrexate, fulvestrant, gefitinib, gemcitabine hydrochloride, gemtuzumab ozogamicin, gemcitabine hydrochloride, afatinib dimaleate, imatinib mesylate, eribulin mesylate, trastuzumab, topotecan hydrochloride, ibritumomab tiuxetan, ponatinib hydrochloride, ifosfamide, imatinib mesylate, imiquimod, axitinib, recombinant interferon alfa-2b, iodine 131 tositumomab and tositumomab, ipilimumab, gefitinib, irinotecan hydrochloride, romidepsin, ixabepilone, ruxolitinib phosphate, cabazitaxel, ado-trastuzumab emtansine, raloxifene hydrochloride, palifermin, carfilzomib, lapatinib ditosylate, lenalidomide, letrozole, leucovorin calcium, leuprolide acetate, lomustine, leuprolide acetate, vincristine sulfate liposome, procarbazine hydrochloride, mechlorethamine hydrochloride, megestrol acetate, megestrol acetate, trametinib, mercaptopurine, mesna, temozolomide, methotrexate, mitomycin, plerixafor, mechlorethamine hydrochloride, mitomycin c, azacitidine, gemtuzumab ozogamicin, nanoparticle paclitaxel, vinorelbine tartrate, nelarabine, filgrastim, sorafenib tosylate, nilotinib, tamoxifen citrate, romiplostim, ofatumumab, omacetaxine mepesuccinate, pegaspargase, denileukin diftitox, oxaliplatin, paclitaxel, paclitaxel albumin-stabilized nanoparticle formulation, palifermin, palonosetron hydrochloride, pamidronate disodium, panitumumab, pazopanib hydrochloride, pegaspargase, peginterferon alfa-2b, pemetrexed disodium, pertuzumab, cisplatin, plerixafor, pomalidomide, ponatinib hydrochloride, pralatrexate, prednisone, procarbazine hydrochloride, aldesleukin, denosumab, eltrombopag olamine, sipuleucel-t, mercaptopurine, radium 223 dichloride, raloxifene hydrochloride, rasburicas, recombinant interferon alfa-2b, regorafenib, lenalidomide, methotrexate, rituximab, romidepsin, romiplostim, daunorubicin hydrochloride, ruxolitinib phosphat, sipuleucel-t, sorafenib tosylate, dasatinib, regorafenib, peginterferon alfa-2b, thalidomide, omacetaxine mepesuccinate, dabrafenib, tamoxifen citrate, cytarabine, erlotinib hydrochloride, bexarotene, nilotinib, docetaxel, temozolomide, temsirolimus, thalidomide, etoposide, topotecan hydrochloride, toremifene, temsirolimus, tositumomab and i 131 iodine tositumomab, dexrazoxane hydrochloride, trametinib, trastuzumab, bendamustine hydrochloride, lapatinib ditosylate, vandetanib, panitumumab, veip, vinblastine sulfate, bortezomib, vemurafenib, etoposide, leuprolide acetate, azacitidine, vincristine sulfate, vinorelbine tartrate, vismodegib, glucarpidase, vorinostat, pazopanib hydrochloride, leucovorin calcium, crizotinib, capecitabine, denosumab, radium 223 dichloride, enzalutamide, ipilimumab, ziv-aflibercept, vemurafenib, ibritumomab tiuxetan, dexrazoxane hydrochloride, ziv-aflibercept, zoledronic acid, vorinostat, zoledronic acid, and abiraterone acetate.

8. The method of claim 4, wherein the cancer therapeutic agent is temozolomide.

9. (canceled)

10. The method of claim 1, wherein the portion of the carboxy terminus consists of the carboxy terminal most 3 to 120 contiguous amino acids from the alpha connexin protein.

11. The method of claim 1, wherein the portion of the carboxy terminus consists of the carboxy terminal most 4 to 30 contiguous amino acids from the alpha connexin protein.

12. The method of claim 1, wherein the portion of the carboxy terminus consists of the carboxy terminal most 5 to 19 contiguous amino acids from the alpha connexin protein.

13. The method of claim 1, wherein the portion of the carboxy terminus consists of the last 9 contiguous amino acids of the carboxy terminus of the alpha connexin protein.

14. The method of claim 1, wherein the alpha connexin protein is selected from the group consisting of Connexin 30.2, Connexin 31.9, Connexin 33, Connexin 35, Connexin 36, Connexin 37, Connexin 38, Connexin 39, Connexin 39.9, Connexin 40, Connexin 40.1, Connexin 43, Connexin 43.4, Connexin 44, Connexin 44.2, Connexin 44.1, Connexin 45, Connexin 46, Connexin 46.6, Connexin 47, Connexin 49, Connexin 50, Connexin 56, and Connexin 59.

15. (canceled)

16. The method of claim 1, wherein the peptide comprises one or more amino acid sequence selected from the group consisting of SEQ ID NO: 1, SEQ ID NO: 2, SEQ ID NO: 3, SEQ ID NO: 4, SEQ ID NO: 5, and SEQ ID NO:43.

17. (canceled)

18. The method of claim 1, wherein the peptide has 1 to 5 conservative substitutions as compared to a polypeptide having the sequence of SEQ ID NO:2.

19. The method of claim 1, wherein the peptide comprises an amino acid sequence with at least 65% sequence identity to the c-terminal most 9 amino acids of SEQ ID NO:1.

20-21. (canceled)

22. The method of claim 1, wherein the peptide further comprises a cellular internalization sequence.

23. The method of claim 22, wherein the cellular internalization sequence comprises an amino acid sequence of one or more protein selected from the group consisting of Antennapedia, TAT, HIV-Tat, Penetratin, Antp-3A (Antp mutant), Buforin II, Transportan, MAP (model amphipathic peptide), K-FGF, Ku70, Prion, pVEC, Pep-1, SynB 1, Pep-7, HN-1, BGSC (Bis-Guanidinium-Spermidine-Cholesterol) and BGTC (Bis-Guanidinium-Tren-Cholesterol).

24. The method of claim 23, wherein the cellular internalization sequence is Antennapedia, and wherein the sequence comprises the amino acid sequence of SEQ ID NO:7.

25. (canceled)

26. The method of claim 22, wherein the peptide is linked at its amino terminus to the cellular internalization sequence, and wherein the amino acid sequence of the polypeptide and cellular internalization sequence has at least 88% sequence identity to SEQ ID NO:8, SEQ ID NO:9, SEQ ID NO:10, SEQ ID NO:11, or SEQ ID NO:12.

27. The method of claim 1, wherein the nucleic acid comprises one or more sequence selected from the group consisting of SEQ ID NO: 13, SEQ ID NO:85, SEQ ID NO:86, SEQ ID NO:87, SEQ ID NO:88, and SEQ ID NO:89.

28-84. (canceled)

85. A vector comprising a nucleic acid encoding a chimeric polypeptide comprising the following components:
a secretory signal peptide;
an Fc fragment;
IL13 peptide;
an MMP cleavage domain;
a cellular internalization peptide; and
a fragment of an alpha connexin protein;
wherein the fragment of an alpha connexin protein represents a contiguous sequence of amino acids representing a portion of the carboxy terminus of an alpha connexin protein or conservative variant thereof wherein said carboxy terminus includes the sequence up to the transmembrane domain.

86-146. (canceled)


SEQUENCE LISTING (92 sequences)

SEQ ID NO: 1 - protein, 20 aa, Artificial Sequence
  synthetic
PSSRASSRASSRPRPDDLEI

SEQ ID NO: 2 - protein, 9 aa, Artificial Sequence
  synthetic
RPRPDDLEI

SEQ ID NO: 3 - protein, 9 aa, Artificial Sequence
  synthetic
RPRPDDLEV

SEQ ID NO: 4 - protein, 9 aa, Artificial Sequence
  synthetic
RPRPDDVPV

SEQ ID NO: 5 - protein, 9 aa, Artificial Sequence
  synthetic
KARSDDLSV

SEQ ID NO: 6 - protein, 27 aa, Artificial Sequence
  synthetic
AGACCTCGGCCTGATGACCTGGAGATT

SEQ ID NO: 7 - protein, 16 aa, Artificial Sequence
  synthetic
RQPKIWFPNRRKPWKK

SEQ ID NO: 8 - protein, 36 aa, Artificial Sequence
  synthetic
RQPKIWFPNRRKPWKKPSSRASSRASSRPRPDDLEI

SEQ ID NO: 9 - protein, 25 aa, Artificial Sequence
  synthetic
RQPKIWFPNRRKPWKKRPRPDDLEI

SEQ ID NO: 10 - protein, 25 aa, Artificial Sequence
  synthetic
RQPKIWFPNRRKPWKKRPRPDDLEV

SEQ ID NO: 11 - protein, 25 aa, Artificial Sequence
  synthetic
RQPKIWFPNRRKPWKKRPRPDDVPV

SEQ ID NO: 12 - protein, 25 aa, Artificial Sequence
  synthetic
RQPKIWFPNRRKPWKKKARSDDLSV

SEQ ID NO: 13 - protein, 74 aa, Artificial Sequence
  synthetic
CGGCAGCCCAAGATCTGGTTCCCCAACCGGCGGAAGCCCTGGAAGAAGCGGCCCGGCCCGACGACCTGGAGATC

SEQ ID NO: 14 - protein, 11 aa, Artificial Sequence
  synthetic
GRKKRRQRPPQ

SEQ ID NO: 15 - protein, 16 aa, Artificial Sequence
  synthetic
RQIKIWFQNRRMKWKK

SEQ ID NO: 16 - protein, 16 aa, Artificial Sequence
  synthetic
RQIAIWFQNRRMKWAA

SEQ ID NO: 17 - protein, 9 aa, Artificial Sequence
  synthetic
RKKRRQRRR

SEQ ID NO: 18 - protein, 21 aa, Artificial Sequence
  synthetic
TRSSRAGLQFPVGRVHRLLRK

SEQ ID NO: 19 - protein, 26 aa, Artificial Sequence
  synthetic
GWTLNSAGYLLGKINKALAALAKKIL

SEQ ID NO: 20 - protein, 18 aa, Artificial Sequence
  synthetic
KLALKLALKALKAALKLA

SEQ ID NO: 21 - protein, 16 aa, Artificial Sequence
  synthetic
AAVALLPAVLLALLAP

SEQ ID NO: 22 - protein, 10 aa, Artificial Sequence
  synthetic
VPMLKPMLKE

SEQ ID NO: 23 - protein, 28 aa, Artificial Sequence
  synthetic
MANLGYWLLALFVTMWTDVGLCKKRPKP

SEQ ID NO: 24 - protein, 18 aa, Artificial Sequence
  synthetic
LLIILRRRIRKQAHAHSK

SEQ ID NO: 25 - protein, 21 aa, Artificial Sequence
  synthetic
KETWWETWWTEWSQPKKKRKV

SEQ ID NO: 26 - protein, 18 aa, Artificial Sequence
  synthetic
RGGRLSYSRRRFSTSTGR

SEQ ID NO: 27 - protein, 15 aa, Artificial Sequence
  synthetic
SDLWEMMMVSLACQY

SEQ ID NO: 28 - protein, 12 aa, Artificial Sequence
  synthetic
TSPLNIHNGQKL

SEQ ID NO: 29 - protein, 19 aa, Artificial Sequence
  synthetic
PSRASSRASSRPRPDDLEI

SEQ ID NO: 30 - protein, 20 aa, Artificial Sequence
  synthetic
GSNKSTASSKSPDPKNSVWI

SEQ ID NO: 31 - protein, 20 aa, Artificial Sequence
  synthetic
GSNKSSASSKSGDGKNSVWI

SEQ ID NO: 32 - protein, 21 aa, Artificial Sequence
  synthetic
GRASKASRASSGRARPEDLAI

SEQ ID NO: 33 - protein, 13 aa, Artificial Sequence
  synthetic
GSASSRDGKTVWI

SEQ ID NO: 34 - protein, 19 aa, Artificial Sequence
  synthetic
PRVSVPNFGRTQSSDSAYV

SEQ ID NO: 35 - protein, 19 aa, Artificial Sequence
  synthetic
PRMSMPNFGRTQSSDSAYV

SEQ ID NO: 36 - protein, 21 aa, Artificial Sequence
  synthetic
PRAGSEKGSASSRDGKTTVWI

SEQ ID NO: 37 - protein, 23 aa, Artificial Sequence
  synthetic
GYHSDKRRLSKASSKARSDDLSV

SEQ ID NO: 38 - protein, 19 aa, Artificial Sequence
  synthetic
PLSRLSKASSRARSDDLTV

SEQ ID NO: 39 - protein, 22 aa, Artificial Sequence
  synthetic
PNHVVSLTNNLIGRRVPTDLQI

SEQ ID NO: 40 - protein, 29 aa, Artificial Sequence
  synthetic
PSCVSSSAVLTTICSSDQVVPVGLSSFYM

SEQ ID NO: 41 - protein, 21 aa, Artificial Sequence
  synthetic
GRSSKASKSSGGRARAADLAI

SEQ ID NO: 42 - protein, 17 aa, Artificial Sequence
  synthetic
LCYLLIRYCSGKSKKPV

SEQ ID NO: 43 - protein, 18 aa, Artificial Sequence
  synthetic
GQKPPSRPSSSASKKQYV

SEQ ID NO: 44 - protein, 19 aa, Artificial Sequence
  synthetic
SSRASSRASSRPRPDDLEV

SEQ ID NO: 45 - protein, 9 aa, Artificial Sequence
  synthetic
RPKPDDLEI

SEQ ID NO: 46 - protein, 19 aa, Artificial Sequence
  synthetic
SSRASSRASSRPKPDDLEI

SEQ ID NO: 47 - protein, 9 aa, Artificial Sequence
  synthetic
RPKPDDLDI

SEQ ID NO: 48 - protein, 19 aa, Artificial Sequence
  synthetic
SSRASSRASSRPRPDDLDI

SEQ ID NO: 49 - protein, 19 aa, Artificial Sequence
  synthetic
SSRASTRASSRPRPDDLEI

SEQ ID NO: 50 - protein, 9 aa, Artificial Sequence
  synthetic
RPRPEDLEI

SEQ ID NO: 51 - protein, 19 aa, Artificial Sequence
  synthetic
SSRASSRASSRPRPEDLEI

SEQ ID NO: 52 - protein, 9 aa, Artificial Sequence
  synthetic
GDGKNSVWV

SEQ ID NO: 53 - protein, 23 aa, Artificial Sequence
  synthetic
SKAGSNKSTASSKSGDGKNSVWV

SEQ ID NO: 54 - protein, 18 aa, Artificial Sequence
  synthetic
GQKPPSRPSSSASKKLYV

SEQ ID NO: 55 - protein, 24 aa, Artificial Sequence
  synthetic
RQPKIWFPNRRKPWKIELDDPRPR

SEQ ID NO: 56 - protein, 20 aa, Artificial Sequence
  synthetic
GRKKRRQRPPQRPRPDDLEI

SEQ ID NO: 57 - protein, 25 aa, Artificial Sequence
  synthetic
RQIKIWFQNRRMKWKKRPRPDDLEI

SEQ ID NO: 58 - protein, 25 aa, Artificial Sequence
  synthetic
RQIAIWFQNRRMKWAARPRPDDLEI

SEQ ID NO: 59 - protein, 18 aa, Artificial Sequence
  synthetic
RKKRRQRRRRPRPDDLEI

SEQ ID NO: 60 - protein, 30 aa, Artificial Sequence
  synthetic
TRSSRAGLQFPVGRVHRLLRKRPRPDDLEI

SEQ ID NO: 61 - protein, 35 aa, Artificial Sequence
  synthetic
GWTLNSAGYLLGKINKALAALAKKILRPRPDDLEI

SEQ ID NO: 62 - protein, 27 aa, Artificial Sequence
  synthetic
KLALKLALKALKAALKLARPRPDDLEI

SEQ ID NO: 63 - protein, 25 aa, Artificial Sequence
  synthetic
AAVALLPAVLLALLAPRPRPDDLEI

SEQ ID NO: 64 - protein, 19 aa, Artificial Sequence
  synthetic
VPMLKPMLKERPRPDDLEI

SEQ ID NO: 65 - protein, 37 aa, Artificial Sequence
  synthetic
MANLGYWLLALFVTMWTDVGLCKKRPKPRPRPDDLEI

SEQ ID NO: 66 - protein, 27 aa, Artificial Sequence
  synthetic
LLIILRRRIRKQAHAHSKRPRPDDLEI

SEQ ID NO: 67 - protein, 30 aa, Artificial Sequence
  synthetic
KETWWETWWTEWSQPKKKRKVRPRPDDLEI

SEQ ID NO: 68 - protein, 27 aa, Artificial Sequence
  synthetic
RGGRLSYSRRRFSTSTGRRPRPDDLEI

SEQ ID NO: 69 - protein, 24 aa, Artificial Sequence
  synthetic
SDLWEMMMVSLACQYRPRPDDLEI

SEQ ID NO: 70 - protein, 21 aa, Artificial Sequence
  synthetic
TSPLNIHNGQKLRPRPDDLEI

SEQ ID NO: 71 - protein, 120 aa, Artificial Sequence
  synthetic
KGRSDPYHATTGPLSPSKDCGSPKYAYFNGCSSPTAPLSPMSPPGYKLVTGDRNNSSCRNYNKQASEQNWANYSAEQNRMGQAGSTISNSHAQPFDFPDDSQNAKKVAAGHELQPLAIVD

SEQ ID NO: 72 - protein, 120 aa, Artificial Sequence
  synthetic
KGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVD

SEQ ID NO: 73 - protein, 120 aa, Artificial Sequence
  synthetic
KTDPYSHSGTMSPSKDCGSPKYAYYNGCSSPTAPLSPMSPPGYKLVTGDRNNSSCRNYNKQASEQNWANYSAEQNRMGQAGSTISNSHAQPFDFADEHQNTKKLASGHELQPLTIVDQRP

SEQ ID NO: 74 - protein, 120 aa, Artificial Sequence
  synthetic
LGFGTIRDSLNSKRRELEDPGAYNYPFTWNTPSAPPGYNIAVKPDQIQYTELSNAKIAYKQNKANTAQEQQYGSHEENLPADLEALQREIRMAQERLDLAVQAYSHQNNPHGPREKKAKV

SEQ ID NO: 75 - protein, 120 aa, Artificial Sequence
  synthetic
GFGTIRDTLNNKRKELEDSGTYNYPFTWNTPSAPPGYNIAVKPDQMQYTELSNAKMAYKQNKANIAQEQQYGSNEENIPADLENLQREIKVAQERLDMAIQAYNNQNNPGSSSREKKSKA

SEQ ID NO: 76 - protein, 120 aa, Artificial Sequence
  synthetic
PYLVDCFVSRPTEKTIFIIFMLVVGLISLVLNLLELVHLLCRCLSRGMRARQGQDAPPTQGTSSDPYTDQVFFYLPVGQGPSSPPCPTYNGLSSSEQNWANLTTEERLASSRPPLFLDPP

SEQ ID NO: 77 - protein, 120 aa, Artificial Sequence
  synthetic
CGSKEHGNRKMRGRLLLTYMASIFFKSVFEVAFLLIQWYLYGFTLSAVYICEQSPCPHRVDCFLSRPTEKTIFILFMLVVSMVSFVLNVIELFYVLFKAIKNHLGNEKEEVYCNPVELQK

SEQ ID NO: 78 - protein, 199 aa, Artificial Sequence
  synthetic
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNH

SEQ ID NO: 79 - DNA, 60 bp, Artificial Sequence
  synthetic
ccctcctcccgggcctcctcccgggcctcctcccggccccggcccgacgacctggagatc

SEQ ID NO: 80 - DNA, 27 bp, Artificial Sequence
  synthetic
cggccccggcccgacgacctggagatc

SEQ ID NO: 81 - DNA, 27 bp, Artificial Sequence
  synthetic
cggccccggcccgacgacctggaggtg

SEQ ID NO: 82 - DNA, 27 bp, Artificial Sequence
  synthetic
cggccccggcccgacgacgtgcccgtg

SEQ ID NO: 83 - DNA, 27 bp, Artificial Sequence
  synthetic
aaggcccggtccgacgacctgtccgtg

SEQ ID NO: 84 - DNA, 48 bp, Artificial Sequence
  synthetic
cggcagcccaagatctggttccccaaccggcggaagccctggaagaag

SEQ ID NO: 85 - DNA, 108 bp, Artificial Sequence
  synthetic
cggcagcccaagatctggttccccaaccggcggaagccctggaagaagccctcctcccgggcctcctcccgggcctcctcccggccccggcccgacgacctggagatc

SEQ ID NO: 86 - DNA, 75 bp, Artificial Sequence
  synthetic
cggcagcccaagatctggttccccaaccggcggaagccctggaagaagcggccccggcccgacgacctggagatc

SEQ ID NO: 87 - DNA, 75 bp, Artificial Sequence
  synthetic
cggcagcccaagatctggttccccaaccggcggaagccctggaagaagcggccccggcccgacgacctggaggtg

SEQ ID NO: 88 - DNA, 75 bp, Artificial Sequence
  synthetic
cggcagcccaagatctggttccccaaccggcggaagccctggaagaagcggccccggcccgacgacgtgcccgtg

SEQ ID NO: 89 - DNA, 75 bp, Artificial Sequence
  synthetic
cggcagcccaagatctggttccccaaccggcggaagccctggaagaagaaggcccggtccgacgacctgtccgtg

SEQ ID NO: 90 - protein, 20 aa, Artificial Sequence
  synthetic
PCSRASSRMSSRARPDDLDV

SEQ ID NO: 91 - protein, 19 aa, Artificial Sequence
  synthetic
PRVSVPNFGRTQSSDSAYV

SEQ ID NO: 92 - protein, 9 aa, Artificial Sequence
  synthetic
RPRPDDELI